(19) United States
(12) Patent Application Publication
(10) Pub. No.: US 2026/0345108 A1
(43) Pub. Date: Nov. 5, 2026

(54) X-RAY UNIT, IMAGING DEVICE, METHOD FOR OPERATING AN X-RAY UNIT, METHOD FOR ACQUIRING IMAGE DATA, AND COMPUTER PROGRAM PRODUCT

(71) Applicant: Siemens Healthineers AG, Forchheim (DE)

(72) Inventors: Joerg FREUDENBERGER, Kalchreuth (DE); Martin BATZKE, Nuernberg (DE); Nico JUNG, Bad Blankenburg (DE); Steffen KAPPLER, Hallerndorf-Pautzfeld (DE); Katrin HALL, Eggolsheim (DE); Marcus RADICKE, Veitsbronn (DE); Stefan SPAETH, Bubenreuth (DE); Sebastian GLAWION, Hausen (DE); Ralf NANKE, Neunkirchen am Brand (DE)

(73) Assignee: Siemens Healthineers AG, Forchheim (DE)

(21) Appl. No.: 19/734,868

(22) Filed: Jul. 8, 2026

Related U.S. Application Data
(63) Continuation of application No. 19/578,169, filed on Mar. 25, 2026.

(30) Foreign Application Priority Data
Mar. 26, 2025 (EP) 25166270.6

(51) Int. Cl. A61B 6/00; A61B 6/06; A61B 6/40

(52) U.S. Cl. CPC A61B 6/54; A61B 6/06; A61B 6/4021; A61B 6/4488

(57) ABSTRACT

An X-ray unit comprises an X-ray tube and a coil. The X-ray tube includes a cathode, a rotating anode, and a vacuum housing. The cathode and the rotating anode are arranged within the vacuum housing. The coil is arranged outside the vacuum housing and electrically isolated from the X-ray tube. The coil is configured to generate a magnetic field, wherein the magnetic field is configured to deflect an electron beam originating from the cathode and impinging on the anode plate in a focal spot of the X-ray tube. The vacuum housing has a beam exit window for passage of X-rays originating from the focal spot on the anode plate.

### CROSS-REFERENCE TO RELATED APPLICATION(S)

The present application is a continuation of U.S. application Ser. No. 19/578,169, filed Mar. 25, 2026, which claims priority under 35 U.S.C. § 119 to European Patent Application No. 25166270.6, filed Mar. 26, 2025, the entire contents of each of which are incorporated herein by reference.

### FIELD

One or more example embodiments of the present invention relate to an X-ray unit, an imaging device, a method for operating an X-ray unit, a method for acquiring image data by way of an imaging device and a non-transitory computer program product or non-transitory computer-readable medium.

One or more example embodiments of the present invention relate to the field of medical imaging, in particular to X-ray units and imaging devices that are used in the context of diagnostics and/or in medical examinations. Such devices are widely used in various fields of medicine, such as for example in mammography, computed tomography, and/or general radiology.

### BACKGROUND

Medical imaging uses X-rays to create captures of an object under examination, for example a human body or body part. This may take place using an X-ray tube that generates X-rays which are then guided through the tissue to be examined of the object under examination and acquired by an X-ray detector. The intensity of the acquired X-rays may vary depending on the density and composition of the irradiated tissue, so giving rise to an image of the internal structures.

Conventional X-ray units typically comprise an X-ray tube with a cathode and an anode that are arranged in a vacuum housing. The cathode emits electrons that are accelerated by a high voltage to the anode. Impingement of the electrons on the anode generates X-rays that exit from the X-ray tube through a beam exit window.

One known problem with conventional X-ray units is limited control over the electron beam within the X-ray tube. This may limit image quality and flexibility in imaging. In particular in advanced imaging techniques such as tomosynthesis, in which multiple captures are made from different angles, precise control of the electron beam may be of great significance.

A focal spot, in particular a region on the anode on which the electron beam impinges and the X-rays are generated may have a decisive impact on image quality. In techniques such as tomosynthesis, the X-ray tube moves relative to the X-ray detector, so resulting in movement of the focal spot relative to the X-ray detector. This movement may cause blur and distortion in the image since the origin of the X-rays changes during capture. This may impair the spatial resolution and contrast of the image data, which is problematic in particular for the identification of fine structures such as microcalcifications.

### SUMMARY

It is therefore an object of one or more example embodiments of the present invention to enable improved image quality in captures involving a moving X-ray unit.

Independent of the grammatical term usage, individuals with male, female or other gender identities are included within the term.

At least the aforementioned object is achieved according to one or more example embodiments of the present invention by the subject matter of the independent claims. Advantageous embodiments with expedient further developments constitute the subject matter of the subclaims.

A first aspect of one or more example embodiments of the present invention relates to an X-ray unit. The X-ray unit comprises an X-ray tube and a coil. The X-ray tube comprises a cathode, a rotating anode and a vacuum housing. The rotating anode comprises an anode plate rotatably bearing-mounted about an anode axis. The rotating anode may be one embodiment of an anode. The cathode and the rotating anode are arranged within the vacuum housing. The coil is arranged outside the vacuum housing. The coil and the X-ray tube are electrically isolated from one another. The coil is configured to generate a magnetic field that permeates an interspace between the cathode and the anode plate. The magnetic field is configured to deflect an electron beam originating from the cathode and impinging on the anode plate in a focal spot of the X-ray tube. The vacuum housing has a beam exit window on a side arranged radially relative to the anode axis. The beam exit window is configured to allow passage of X-rays originating from the focal spot on the anode plate.

The X-ray unit, in particular also denoted X-ray source, may be an apparatus that is configured to generate and emit X-rays. The X-ray unit may comprise various components that interact to generate and control the X-rays.

The X-ray tube may be configured as a vacuum tube for generating X-rays. The X-ray tube comprises the cathode, the rotating anode and the vacuum housing.

The cathode may be configured as an electrode that is configured to emit electrons, in particular the electron beam. The cathode may be produced, for example, from a material such as tungsten. In addition, the cathode may be configured to liberate electrons by electrical heating. The cathode may have an electron emission surface oriented toward the anode plate. The electron beam may take the form of a directed stream of electrons that is emitted by the cathode and accelerated to the anode plate. It is not necessary for all the electrons emitted by the cathode to impinge on the anode plate within a defined spatial region, in particular a defined area. The focal spot may be configured as the spatial region on the anode plate on which the electron beam impinges and where the X-rays are generated. A size and/or shape of the focal spot may influence the quality of the generated X-rays. Some of the electrons emitted by the cathode may arrive on the anode plate outside the focal spot due to various factors such as scattering, deflection by magnetic fields, and/or thermal motion.

The rotating anode may be configured as a rotatable anode, in particular a rotatable anode plate, that is configured to capture the electrons emitted by the cathode and generate X-rays. X-rays may take the form of electromagnetic radiation with wavelengths in the range from around 0.002 to 1 nanometer. The X-rays may be generated by the deceleration of high-energy electrons in the anode material of the anode plate. The rotating anode comprises the anode plate that is rotatably bearing-mounted about the anode axis. The anode plate may take the form of a disk-shaped structure that is a target area for the electron beam. The anode plate may in particular be rotationally symmetrical relative to a center of rotation. The anode plate may be attached to one end of a shaft which runs along the anode axis. The anode plate may be produced from a base member, in particular comprising a molybdenum alloy, with a coating of a high-melting material such as tungsten or a tungsten-rhenium alloy. The anode axis may take the form of an axis of rotation of the anode plate about which the anode plate is rotatably bearing-mounted. The anode axis may run through an in particular geometric center point of the anode plate and perpendicularly to a surface, in particular plane, of the disk-shaped structure of the anode plate. The anode plate may advantageously rotate about the anode axis when the X-ray unit is in an operating state.

The vacuum housing may be configured as an air-tight housing that encloses the cathode and the rotating anode and maintains a vacuum, in particular a high vacuum, when the X-ray unit is in an operating state. The vacuum housing may be produced from a material such as glass, ceramic, and/or metal. The vacuum housing may be configured to maintain a vacuum in the interior and to enclose the components of the X-ray tube. The vacuum housing may have a complex three-dimensional shape that may be configured to accommodate the internal components of the X-ray tube and simultaneously maintain vacuum integrity. For example, the vacuum housing may have a substantially cylindrical main body with one or more projections or widened portions for accommodating specific components. The cylindrical main body of the vacuum housing may extend along a longitudinal axis of the X-ray tube. The vacuum housing may have a cathode housing portion at one end of the cylindrical main body. The cathode housing portion may be somewhat wider than the main body and have a dome-shaped or hemispherical end cap. The vacuum housing may have an anode housing portion at an opposite end of the cylindrical main body. The anode housing portion may be characterized by a conical or tapering shape that narrows toward the end. The anode housing portion may terminate in a flat or slightly rounded end face. The vacuum housing may have a lateral extension or convexity for accommodating the beam exit window. This extension may be arranged radially to the anode axis and protrude from the cylindrical main body. Viewed from the side, this extension may be rectangular or trapezoidal in shape, wherein the dimensions are adapted to the size of the beam exit window.

The vacuum housing may furthermore have cooling fins and/or channels on its outer face, in particular in regions close to the rotating anode. These fins may be arranged circularly around the main body or in a spiral pattern.

The vacuum housing may furthermore have attachment points or flanges at various locations, for example a circular flange at a cathode-side end of the vacuum housing and similar attachment points at the anode-side end of the vacuum housing, in particular for securing the rotating anode and/or an associated rotation mechanism.

A surface of the vacuum housing may be smooth and polished in order to minimize outgassing and maintain vacuum integrity. In some regions of the vacuum housing in which the coil is arranged externally, the vacuum housing may have a reduced wall thickness or be manufactured from a material that enables efficient permeation of the magnetic field.

An overall shape and/or dimensions of the vacuum housing may be optimized in order to reconcile factors such as internal volume, surface area for heat dissipation, mechanical strength, and/or compatibility with external components, for example the coil and/or any shielding or cooling systems.

The coil may take the form of an electrical component. The coil may be wound from an electrically conductive material, for example copper wire. The coil may be configured to generate the magnetic field. The magnetic field may take the form of a region in which magnetic forces act. The coil is configured to generate a magnetic field in such a way that it permeates the interspace between the cathode and the anode plate. The interspace between the cathode and the anode plate may take the form of the spatial region within the X-ray tube through which the electron beam runs from the cathode to the anode plate. The magnetic field that can be generated by the coil may be configured to influence, in particular deflect, the electron beam in its trajectory. Deflection of the electron beam may take the form of a modification to the trajectory of the electron beam by the magnetic field. Deflection may serve to control the position of the focal spot on the anode plate. The strength of the magnetic field and thus the deflection of the electron beam may be controlled by varying the energization of the coil. The magnetic field is configured to deflect the electron beam originating from the cathode and impinging on the anode plate in a focal spot of the X-ray tube.

The coil and the X-ray tube are electrically isolated from one another. The electrical isolation may take the form of electrical insulation between the coil and the X-ray tube. In the context of the X-ray unit, this means that the coil and the X-ray tube are electrically insulated from one another. Due to the electrical isolation between the X-ray tube and the coil, it is advantageously possible to avoid influence, in particular mutual influence, between the high voltage in the X-ray tube and the coil, in particular the magnetic field of the coil. In particular, due to the electrical isolation, arcing in the X-ray tube may advantageously have no influence on deflection of the electron beam and deflection of the electron beam may have no influence on the high-voltage stability of the X-ray tube in the event of deflection.

The coil, which is configured as a deflection coil for the electron beam, is arranged outside the vacuum housing of the X-ray tube and is advantageously not directly electrically connected to the components of the X-ray tube. Insulation effort within the X-ray tube may consequently be reduced as no additional high-voltage insulators are required for the coil. The coil may thus be located on a separate “insulation island” that is electrically independent of the X-ray tube.

Electrical isolation may be achieved, for example, by an air gap and/or an insulating material between the coil and the vacuum housing of the X-ray tube. The air gap and/or the insulation material may be dimensioned such that they ensure the necessary electrical insulation while simultaneously enabling effective magnetic coupling for deflection of the electron beam.

In order to maintain electrical isolation, the coil may be supplied, in particular energized, by a separate power supply unit that operates independently of a high-voltage supply of the X-ray tube. Control signals for the coil may be transferred via optical and/or capacitive couplers in order to ensure complete electrical insulation.

The coil may have an opening region, in particular an opening and/or hole that is configured to accommodate at least part of the X-ray tube, in particular the vacuum housing. A diameter of the opening region may be the same as or minimally larger than an external diameter of the vacuum housing in order to minimize the distance between the coil and the electron beam. The coil may advantageously be shaped such that it closely follows the outlines of the X-ray tube, in particular of the vacuum housing, which may improve the efficiency of magnetic field interaction with the electron beam. The coil may have a cylindrical shape or a complex geometry. The coil may advantageously have one or more portions, in particular segments. If the coil has multiple portions, the multiple portions may be mutually independently controllable in order to enable more precise handling of the magnetic field. The coil may advantageously have an axial extent such that the magnetic field that can be generated sufficiently covers the entire path of the electron beam from the cathode to the anode plate. The coil may advantageously be encapsulated and/or potted, for example in a resin, in a housing. This housing may also serve to protect the coil from external damage and contamination. The coil may furthermore be mounted on a support structure that enables fine adjustment of the position of the coil relative to the X-ray tube. This adjustability may enable the magnetic field to be precisely oriented relative to the electron beam, which may improve deflection accuracy.

The beam exit window, in particular also denoted exit window, may take the form of a region of the vacuum housing that is transmissive to X-rays. In particular, the beam exit window may be configured to allow passage of X-rays originating from the focal spot on the anode plate. The beam exit window may be produced from a material that has high transmission for X-rays, for example beryllium, a thin metal foil, and/or a composite material. The beam exit window is arranged on a side arranged radially relative to the anode axis. The radial arrangement of the beam exit window relative to the anode axis may comprise various configurations. The beam exit window may be arranged in a position that is located laterally of the anode axis. For example, the passage direction of the beam exit window may be arranged at an angle of between 60 and 120°, in particular 90°, to the anode axis. The beam exit window need not necessarily be oriented parallel to the anode axis. This configuration may enable the X-rays generated by the focal spot on the anode plate to effectively exit from the vacuum housing without the window having to be located directly in the extension of the anode axis or precisely in a direction perpendicular to the anode axis.

The coil may advantageously be arranged relative to the X-ray tube, in particular the vacuum housing, such that the opening region of the coil accommodates at least some of the region of the X-ray tube, in particular of the vacuum housing, in which the X-rays run from the focal spot on the anode to the beam exit window. In particular, the coil may be arranged on the same side, arranged radially relative to the anode axis, as the beam exit window.

The beam exit window may have a planar or curved shape. The curvature may be convex, concave or more complex in shape in order to achieve specific radiation characteristics and/or improve the structural integrity of the window.

The proposed X-ray unit may enable precise control of the focal spot on the anode plate without impairing the vacuum integrity of the X-ray tube. This may help to improve image quality and flexibility in imaging methods.

The X-ray unit may furthermore comprise an X-ray filter. The X-ray filter may be configured to absorb low-energy fractions of the X-rays and/or to optimize the X-ray spectrum of the X-rays. The X-ray filter may comprise aluminum, copper, titanium, rhodium, silver, and/or molybdenum. The X-ray filter may be arranged between the focal spot on the anode plate and the beam exit window, for example on or in a aperture unit. This may enable precise filtering of the X-rays immediately after generation of the X-rays in the focal spot. As a result, the X-ray filter may effectively filter out low-energy photons before the X-rays pass through the beam exit window.

The X-ray filter may enable more uniform irradiation of a target region and a reduction in image noise, resulting in clearer and more diagnostically meaningful X-ray images. In addition, a radiation dose for an object under examination to be examined may advantageously be reduced.

The object under examination may, for example, be a human and/or animal patient and/or an examination phantom.

According to one advantageous embodiment, the X-ray unit may have respective power supply units for separately energizing the X-ray tube and the coil. This configuration may enable more precise control of the individual components.

The power supply unit for energizing the X-ray tube may comprise a high-voltage source that may be configured to provide a voltage between the cathode and rotating anode in the range from around 10 kV to 300 kV. This high-voltage source may, for example, comprise a high-voltage transformer, a rectifier and a voltage regulator. In particular, the high-voltage source may be embodied as a resonant converter in order to achieve high efficiency and low output voltage ripple. In addition to the high-voltage supply, the power supply unit for energizing the X-ray tube may comprise a separate low-voltage source for heating the cathode. This heating power supply may provide, for example, 10 mA to 20000 mA depending on the specific requirements of the cathode.

The power supply unit for energizing the coil may be embodied as a separate unit. The power supply unit for energizing the coil may be configured to provide a precise, controllable current. The power supply unit for energizing the coil may comprise, for example, a DC converter with an output voltage of 0 V to 50 V and a maximum current intensity of 0 A to 20 A. In particular, the coil power supply for energizing the coil may be embodied as a four-quadrant power source in order to enable rapid changes in the magnetic field.

Separation of the power supply units for X-ray tube and coil may offer a number of technical advantages. Firstly, it may minimize the mutual influencing of the circuits, which may result in improved stability and accuracy of electron beam control. The separate power supplies may make it possible to operate the coil independently of the high voltage of the X-ray tube, which allows more flexible control of the magnetic field. Furthermore, separation of the power supplies may help to reduce electromagnetic interference. The high-voltage supply of the X-ray tube may potentially cause disturbance that might impair precise control of the deflection current. This risk may be minimized by using separate, shielded power supply units.

Advantageously, the power supply units may each be equipped with digital control interfaces that enable precise and rapid adaptation of the operating parameters. This may be achieved, for example, by using digital signal processors (DSPs) or field-programmable gate arrays (FPGAs) for real-time control of respective energization.

Overall, using separate power supply units for the X-ray tube and the coil may help to improve the performance, stability and flexibility of the X-ray unit. The possibility of mutually independently optimizing the operating parameters of the individual components may improve image quality.

In a further advantageous embodiment of the proposed X-ray unit, the anode plate may take the form of a single-angle plate.

A single-angle plate may denote an anode plate that has a single inclined surface. The inclined surface may comprise the target region for the impinging electron beam. The inclined surface may comprise an annular and/or strip-shaped portion on a side of the anode plate that faces the cathode. The anode plate may advantageously be rotationally symmetrical relative to a center of rotation. The inclined surface may run in a straight line in a radial cross-section of the single-angle plate. The angle of the single-angle plate may be denoted the anode angle. The anode angle may denote an angle between the inclined surface and a plane of rotation of the anode plate, wherein the plane of rotation of the anode plate runs perpendicular to the anode axis. This angle may be for example between 7° and 20°.

The single-angle plate, in particular the angle of the inclined surface, may enable targeted orientation of the generated X-rays. This may be advantageous for orienting the X-ray beam, in particular the X-rays, onto the beam exit window and downstream components such as an X-ray detector.

Using a single-angle plate may enable uniform heat distribution on the anode surface as the electron beam impinges on a larger area. This may extend a service life of the anode plate.

Configuration as a single-angle plate may also influence focusing of the X-rays. In particular, the single-angle plate may help to optimize the size of the focal spot. An effective size of the focal spot, which may have an impact on image quality, may be influenced by the angle of the inclined surface. Due to the angle, the focal spot on the rotating anode may appear to be reduced in size when viewed from the beam exit window, which may help to improve image quality.

The single-angle plate may be manufactured from a high-melting material that has elevated thermal stability and good thermal conductivity. Possible materials comprise for example tungsten, molybdenum or alloys of these metals, for example a tungsten-rhenium alloy. The inclined surface of the single-angle plate may additionally be coated with a thin layer of a material with a high atomic number, such as for example pure tungsten, in order to enable efficient X-ray generation.

In a further advantageous embodiment of the proposed X-ray unit, the single-angle plate may have an angle of 7 to 20°, in particular 16°.

The single-angle plate, in particular the inclined surface, may advantageously have an angle, in particular an anode angle, of 7 to 20°, in particular 16°, in particular relative to the plane of rotation of the single-angle plate. This anode angle may help to focus, in particular orient, the X-rays effectively onto the beam exit window and, simultaneously, to ensure uniform distribution of the heat on the surface of the single-angle plate. The electrons impinging on the anode plate may release their energy over a larger area, which may result in improved heat distribution.

The anode angle of 7 to 20°, in particular 16°, may be advantageous for achieving an optimal distribution of the X-rays on the beam exit window. The anode angle of 7 to 20°, in particular 16°, may advantageously furthermore help to minimize a heel effect that may result in a non-uniform distribution of X-ray intensity. The heel effect describes a decrease in X-ray intensity on the anode-side edge of the radiation field. Furthermore, the anode angle of 7 to 20°, in particular 16°, may enable efficient use of the focal spot, which may help to improve image quality. The anode angle of 7 to 20°, in particular 16°, allows the focal spot on the anode plate to be inclined toward the beam exit window. This may result in the X-rays originating from the focal spot being able to exit more efficiently through the beam exit window.

In a further advantageous embodiment of the proposed X-ray unit, the anode plate may have a diameter of 50 mm to 200 mm, in particular 90 mm.

The anode plate may have a diameter of 50 mm to 200 mm, in particular 90 mm. The diameter of the anode plate may be defined as the maximum distance between two points on an outer edge of the anode plate. An anode plate with a diameter of 50 mm to 200 mm, in particular 90 mm, may be a well-balanced solution that enables efficient X-ray generation with a simultaneously compact construction of the X-ray unit.

In a further advantageous embodiment of the proposed X-ray unit, the anode plate may comprise a support substrate. A layer of a further substrate may be applied on a side facing the cathode.

The anode plate may comprise a support substrate. The support substrate may take the form of the base member or base structure of the anode plate. The support substrate may be manufactured from a material with elevated mechanical strength and good thermal conductivity in order effectively to dissipate the heat arising during X-ray generation. The support substrate may comprise, for example, a metal alloy or a composite material.

A layer of the further substrate may be applied on a side of the support substrate facing the cathode. This layer may take the form of the target layer or active layer of the anode plate on which the electron beam impinges and X-rays are generated. The further substrate may be manufactured of a material with a high atomic number in order to enable efficient X-ray generation. The layer of the further substrate may have been applied onto the support substrate using various coating methods such as vapor deposition, sputtering, and/or electrodeposition.

If the anode plate is configured as a single-angle plate, the target layer may be arranged at, in particular on, the inclined surface. The target layer may be annular or strip-shaped. This configuration may enable optimized distribution of the further substrate on the anode surface. The annular or strip-shaped arrangement of the target layer may help to distribute thermal loading more uniformly and improve X-ray generation efficiency.

Arranging a support substrate with a layer of a further substrate applied thereto may be advantageous as it enables the positive properties of the two materials to be combined. The support substrate may provide mechanical stability and heat dissipation, while the layer of the further substrate may have improved properties for X-ray generation.

In a further advantageous embodiment of the proposed X-ray unit, the support substrate may comprise titanium-zirconium-molybdenum.

Titanium-zirconium-molybdenum may take the form of an alloy that contains titanium, zirconium and molybdenum. Titanium-zirconium-molybdenum may have advantageous properties for use as a support substrate of an anode plate. This alloy may exhibit elevated thermal conductivity, good mechanical stability and resistance to thermal deformation at high temperatures. Titanium-zirconium-molybdenum may additionally enable good adhesion for further coatings. Using titanium-zirconium-molybdenum as a support substrate may improve the service life and performance of the anode plate. The support substrate may be configured to ensure the mechanical stability of the anode plate and slight thermal expansion and to enable efficient heat dissipation.

The good mechanical stability of titanium-zirconium-molybdenum alloy may enable an elevated loading capacity of the anode plate at the rotational speeds and thermal loads that occur. This may help to minimize deformation and/or damage to the anode plate during operation.

The low thermal expansion of the titanium-zirconium-molybdenum alloy may reduce thermal stresses in the anode plate. This may help to prevent cracking and/or deformation of the anode plate due to changes in temperature.

In addition, the titanium-zirconium-molybdenum alloy may have good resistance to oxidation and corrosion. This may improve the long-term stability of the anode plate under the operating conditions in the X-ray tube.

Using titanium-zirconium-molybdenum as the support substrate may thus enable various advantageous properties for the anode plate. These properties may help to improve the performance, reliability and service life of the X-ray tube.

In a further advantageous embodiment of the proposed X-ray unit, the further substrate may comprise a tungsten-rhenium alloy.

The tungsten-rhenium alloy may be used as a coating material for the anode plate, in particular the target layer of the anode plate, of the X-ray tube. The tungsten-rhenium alloy may, for example, have a rhenium content of up to 30%, in particular between 5 and 12.5%. The rhenium content may advantageously increase thermal and mechanical loading capacity of the coating. The rhenium addition may furthermore increase ductility and toughness of the alloy in comparison with pure tungsten. The tungsten-rhenium alloy may have been applied as a thin target layer onto the support substrate of the anode, for example by way of plasma spraying, sputtering, and/or chemical vapor deposition. The thickness of the target layer may be between 5 μm and 1000 μm. Due to its elevated melting temperature, good thermal conductivity and elevated density, the tungsten-rhenium alloy may serve as a target layer for generating X-rays. The service life and performance of the anode plate may be improved by using a tungsten-rhenium alloy as a further substrate.

The elevated toughness of the tungsten-rhenium alloy in comparison with pure tungsten may extend the service life of the anode plate since cracking and flaking of the coating may be reduced. The good thermal conductivity of the tungsten-rhenium alloy may help to improve the efficiency of heat dissipation from the focal spot, which may enable higher power densities. The elevated density of the tungsten-rhenium alloy may ensure a good X-ray yield.

In a further advantageous embodiment of the proposed X-ray unit, the cathode and the anode plate may be at an average distance of 5 mm to 50 mm, in particular 8 mm.

The average distance may be taken to be a mean or typical distance between the cathode and the anode plate. This distance may advantageously be selected such that it enables optimum focusing of the electron beam on the anode plate. This distance advantageously enables efficient acceleration of the electrons from the cathode to the anode plate, while simultaneously providing sufficient space for deflection of the electron beam by the magnetic field of the coil.

The average distance of 5 mm to 50 mm, in particular 8 mm, may offer various technical advantages. On the one hand, this distance may ensure sufficient electrical insulation between cathode and rotating anode, in particular the anode plate, in order to avoid voltage flashovers. On the other hand, this distance may enable efficient focusing of the electron beam onto the anode plate, which may help to improve image quality.

The average distance selected may also influence the size and geometry of the focal spot. A suitable distance may help to produce a focal spot that is as small and sharply defined as possible, which may be advantageous for example for an elevated spatial resolution of the image data, in particular X-ray image data, that can be generated.

The average distance of 5 mm to 50 mm, in particular 8 mm, may be related to other parameters of the X-ray tube, such as for example the applied high voltage, the geometry of the cathode and/or of the anode plate and the strength of the magnetic field generated for deflecting the electron beam. These parameters may be coordinated with one another to achieve optimum X-ray tube performance.

The average distance of 5 mm to 50 mm, in particular 8 mm, may be achieved by various structural measures in the X-ray tube. For example, the cathode may be mounted on an appropriate mount within the vacuum housing that ensures the desired distance from the anode plate. A bearing mount of the anode plate may likewise be designed such that the necessary distance from the cathode is maintained.

It should be noted that the actual distance between cathode and anode plate may vary slightly at various points, for example due to the rotation of the anode plate or thermal expansion during operation. The stated average distance of 5 mm to 50 mm, in particular 8 mm, is therefore a mean value which is of relevance for the design and functioning of the X-ray tubes.

Maintenance of this specific distance may be ensured by various components and mechanisms within the X-ray tube, for example precise positioning elements, spacers, and/or special mounting techniques for the cathode and anode plate.

The average distance of 5 mm to 50 mm, in particular 8 mm, between cathode and anode plate may advantageously help to improve X-ray generation efficiency. This optimized distance may enable precise control of the electron beam, which may in turn result in higher image quality while simultaneously reducing the radiation exposure of the object under examination. In addition, this specific distance may help to reduce the evolution of heat in the X-ray tube and so extend the service life of the components.

In a further advantageous embodiment of the proposed X-ray unit, the vacuum housing may have respective feedthroughs for energizing the cathode and the rotating anode.

A feedthrough may be configured as an electrically conductive connection that is passed through the vacuum housing. The feedthroughs may be configured to produce an electrical connection between components within the vacuum housing and an external power supply, in particular a respective external current supply. In particular, the feedthroughs may enable electrical contacting of the cathode and rotating anode from outside the vacuum housing.

The feedthroughs for energizing the cathode may be configured to provide a heating current for the cathode. This heating current may heat the cathode to a temperature at which electron emission is enabled.

The feedthroughs for energizing the rotating anode may be configured to apply a high voltage to the rotating anode, in particular to adjust the rotating anode to a positive electrical potential compared to the cathode. This high voltage may accelerate the electrons emitted by the cathode toward the rotating anode.

The feedthroughs may, for example, take the form of metallic conductors that are run through vacuum-tight insulators in the vacuum housing. The conductors may comprise, for example, copper, silver or other highly conductive metals. The conductors may be sufficiently thick to conduct the necessary current without excessive heating. The insulators may consist of ceramics, glass or other vacuum-capable insulation materials.

The feedthroughs may be so dimensioned and arranged as to withstand the elevated voltages and currents in an X-ray tube. It addition, they may be designed such that they do not impair the vacuum tightness of the housing.

Using separate feedthroughs for the cathode and rotating anode may enable independent control of and supply to these components. This may help to improve control and stability of the electron beam and thus of X-ray generation.

The feedthroughs may be connected within the vacuum housing to corresponding connection points on the cathode and rotating anode. Outside the vacuum housing, the feedthroughs may be connectable to a respective external current supply.

Using feedthroughs for energizing the cathode and rotating anode may offer a number of technical advantages. On the one hand, such use enables reliable and efficient power supply of the components within the vacuum housing. On the other hand, the vacuum-tight embodiment of the feedthroughs may ensure the integrity of the vacuum in the interior of the housing. This may help to improve the service life and performance of the X-ray tube.

The feedthroughs, in particular at least the feedthroughs for energizing the cathode, may advantageously be arranged on a cathode-side wall of the vacuum housing. This positioning may be advantageous because it enables a short conduction path to the cathode and simultaneously offers a sufficient distance from the rotating anode to avoid electrical arcing. The feedthroughs for energizing the anode may furthermore be arranged on the anode side relative to the vacuum housing.

The distance between adjacent feedthroughs may be selected such that it corresponds to at least 1.5 times the diameter of an individual feedthrough. This minimum distance may help to prevent electrical arcing between the feedthroughs.

In a further advantageous embodiment of the proposed X-ray unit, the vacuum housing may be formed from a substrate that comprises a glass-ceramic.

A glass-ceramic may be defined as a material that has both vitreous and crystalline properties. Glass-ceramics may be produced by controlled crystallization of glasses. The glass-ceramic substrate may have advantageous properties such as elevated mechanical strength, low thermal expansion and good electrical insulation properties. These properties may be advantageous for use as a vacuum housing for an X-ray tube.

Examples of glass-ceramics that may be used as a substrate for the vacuum housing comprise lithium aluminosilicate glass-ceramics, magnesium aluminosilicate glass-ceramics, borosilicate glass, and/or zinc aluminosilicate glass-ceramics. The precise composition of the glass-ceramic may be adapted to the specific requirements of the X-ray tube.

The vacuum housing made from the substrate comprising a glass-ceramic may have been produced by various methods. For example, a glass blank may first be shaped and then subjected to controlled heat treatment in order to achieve partial crystallization. Alternatively, the vacuum housing may also have been produced by sintering glass-ceramic powders.

Using a substrate of glass-ceramic for the vacuum housing may offer a number of advantages in comparison with conventional materials such as metals or pure ceramics. The glass-ceramic may enable improved vacuum tightness and simultaneously ensure good electrical insulation between the components in the interior of the X-ray tube. Furthermore, the low thermal expansion of the glass-ceramic may help to reduce thermal stresses in the vacuum housing, which may increase the service life and reliability of the X-ray tube. The glass-ceramic may also exhibit elevated resistance to X-rays, which may extend the service life of the vacuum housing. A further advantage of using glass-ceramic as the substrate for the vacuum housing may be the possibility of producing complex shapes and structures. This may make it easier to integrate leadthroughs for electrical terminals, in particular feedthroughs, and/or cooling ducts.

In summary, using a vacuum housing of a substrate that comprises a glass-ceramic may help to improve the performance, reliability, and durability of the X-ray tube.

In a further advantageous embodiment of the proposed X-ray unit, the X-ray unit may further comprise a leak-tight single-tank housing. The X-ray tube and the coil may be arranged within the single-tank housing. The single-tank housing may be filled with a cooling/insulating medium.

The leak-tight single-tank housing may take the form of a closed housing that is configured to prevent the escape of liquids or gases. The leak-tight single-tank housing may be produced from various materials, for example of metal, in particular aluminum or stainless steel, plastics material, a composite material, or a combination thereof. The single-tank housing may have various sealing elements such as O-rings and/or flat gaskets in order the ensure leak-tightness. The leak-tight single-tank housing may additionally be lined with a radiation-absorbing material. The radiation-absorbing material may comprise lead or a composite material comprising a plastics material and a metal.

The X-ray tube and the coil may be arranged within the single-tank housing. This means that both the X-ray tube and the coil may be completely enclosed by the single-tank housing. Arrangement within the single-tank housing may advantageously make it possible to protect the X-ray tube and coil from external influences and to provide controlled surroundings for the operation thereof.

The single-tank housing may be filled with a cooling/insulating medium. A cooling/insulating medium may be defined as a substance that has both cooling and insulating properties. The cooling/insulating medium may comprise a liquid, a gas, or a mixture thereof. Examples of possible cooling/insulating media comprise oils, gases, in particular sulfur hexafluoride, or special dielectric liquids.

The cooling/insulating medium may be configured to absorb and dissipate heat from the components in the interior of the single-tank housing in order to prevent overheating. The cooling/insulating medium may furthermore take the form of an electrical insulator in order to prevent voltage flashovers between the components. The cooling/insulating medium may additionally have corrosion-inhibiting properties and so extend the service life of the components. The cooling/insulating medium may furthermore be configured to improve heat transfer between the components and the single-tank housing.

Using a leak-tight single-tank housing in which the X-ray tube and the coil are arranged and which is filled with a cooling/insulating medium may help to ensure a compact and efficient construction of the X-ray unit. This configuration may enable improved cooling, electrical insulation, and protection of the components, which may result in enhanced performance and reliability of the X-ray unit.

The single-tank housing may, for example, be of a cylindrical basic shape. The single-tank housing may have cooling fins on its outside, in particular cooling fins may be attached to the outside of the single-tank housing, said cooling fins being configured to dissipate heat. The single-tank housing may have ports for filling the single-tank housing with and emptying it of the cooling/insulating medium. These ports may be located for example at an upper and lower end of the single-tank housing in order to enable complete filling and emptying.

The single-tank housing may furthermore have apparatuses for mounting and orienting the internal components, for example mounts and/or rails, which enable precise positioning of the X-ray tube and coil.

The single-tank housing may comprise sensors for monitoring the operating parameters. The sensors may be configured to acquire a temperature, a pressure, and/or a filling level of the cooling/insulating medium. The sensors may be arranged at differing points in the single-tank housing in order to ensure comprehensive monitoring.

In a further advantageous embodiment of the proposed X-ray unit, the X-ray unit may furthermore comprise a high-voltage unit. The high-voltage unit may be configured to provide a high voltage to the X-ray tube. The high-voltage unit may additionally be arranged within the single-tank housing.

The high-voltage unit may, for example, comprise a high-voltage generator that is configured to generate a high voltage of from several kilovolts up to several hundred kilovolts, for example in the range from 10 kV to 300 kV. The high-voltage unit may further comprise control electronics that are configured to regulate the generated high voltage and provide it to the X-ray tube.

The high-voltage unit may additionally be arranged within the single-tank housing. A compact construction of the X-ray unit may be achieved by arranging the high-voltage unit within the single-tank housing. The high-voltage unit may furthermore be protected from external influences by the single-tank housing. In addition, the cooling/insulating medium may cool and electrically insulate the high-voltage unit in the single-tank housing.

Arranging the high-voltage unit within the single-tank housing may offer various advantages. On the one hand, better electromagnetic shielding may thereby be achieved as the single-tank housing may act as a Faraday cage. On the other hand, the spatial vicinity to the X-ray tube may enable shorter high-voltage lines, which may reduce losses and boost efficiency.

In a further advantageous embodiment of the proposed X-ray unit, the single-tank housing may have respective feedthroughs for providing energization to the high-voltage unit and the coil.

The feedthroughs may take the form of electrically insulated leadthroughs through the single-tank housing. The feedthroughs may, for example, take the form of cable leadthroughs, cable entries, and/or cable glands.

Providing energization to the high-voltage unit and the coil may comprise supplying electric current respectively to the high-voltage unit and the coil. Energization may, for example, then be performed by connection to a power source or by actuation of a switch.

The single-tank housing with the respective feedthroughs may be configured to enable a reliable and efficient current supply to the high-voltage unit and the coil and simultaneously to ensure sealing of the housing. The feedthroughs may be designed such that they comply with the specific requirements for voltage, current, and insulation for the high-voltage unit and the coil.

Using feedthroughs to provide energization may have the advantage that a secure and reliable electrical connection may be produced between the external power sources and the internal components of the X-ray unit without impairing the integrity of the single-tank housing.

The feedthroughs for energizing the high-voltage unit may, for example, comprise cables that are designed for the appropriate high voltages. These cables may be run through special leadthroughs, in particular cable leadthroughs, in the single-tank housing.

The feedthroughs for energizing the coil may comprise cables with a suitable cross-section for transferring the necessary current intensities for generating the magnetic field. These cables may be run through separate leadthroughs in the single-tank housing.

The feedthroughs may be arranged and embodied such that they ensure electrical isolation between the high-voltage unit, the coil, and the single-tank housing. This may be achieved by suitable insulation materials and spacing.

In addition to the feedthroughs for the electrical supply, further leadthroughs for control and/or measurement lines may be provided, for example for monitoring and closed-loop control of the components in the interior of the single-tank housing.

The separate feedthroughs for the high-voltage unit and the coil may advantageously minimize possible interference between the various electrical systems. This may enable precise open-and closed-loop control both of the high voltage for the X-ray tube and of the magnetic field for electron beam deflection.

In a further advantageous embodiment of the proposed X-ray unit, the X-ray unit may furthermore comprise a aperture unit, in particular a collimator unit, that is configured to spatially limit the X-rays exiting from the beam exit window.

The aperture unit may comprise mobile apertures, in particular aperture blades, that are configured to limit and adapt a cross-section of the exiting X-rays. In particular, the aperture unit may be configured to collimate the exiting X-rays. The apertures may be manufactured from a radiation-absorbing material, for example lead, steel, or tungsten.

The aperture unit may be configured to be motor-adjustable in order to enable adaptation of the beam limits during operation of the X-ray unit. The aperture unit may be controlled by a control unit of the X-ray unit or of an imaging device comprising the X-ray unit. The aperture unit may be arranged downstream of the beam exit window relative to the direction of the X-rays, in particular between the beam exit window and an object under examination to be mapped. The size of the aperture unit may be dimensioned such that it covers the entire cross-section of the X-ray beam, in particular of the X-rays.

The spatial limitation of the X-rays by the aperture unit may advantageously limit the radiation exposure of the object under examination to a relevant region and improve image contrast by reducing stray radiation.

In a further advantageous embodiment of the proposed X-ray unit, the anode axis may be at a specified angle, in particular 6°, relative to an aperture plane of the aperture unit.

The aperture plane may denote a plane in which the aperture unit is located, in particular in which the apertures or aperture blades of the aperture unit are arranged and/or displaceable, or to which the aperture unit relates. In particular, the aperture plane may denote a cross-sectional plane of an opening region of the aperture unit.

The specified angle between the anode axis and the aperture plane may enable an improved orientation of the X-rays relative to the aperture unit. The specified angle may advantageously be specified such that, relative to the aperture plane, the anode axis is arranged falling on the cathode side and rising on the anode side. The specified angle may make it possible to optimize beam geometry for specific applications, such as for example for mammography or other specialized radiological examinations.

The specified angle of 6° may be taken to be an example of an advantageous angle. Other angles between 0 and 30° may likewise be used, depending on the specific requirements and geometry of the X-ray unit. The specified angle between the anode axis and the aperture plane may serve to enable optimized orientation of the X-rays. This arrangement may achieve improved beam guidance and/or focusing, which may result in enhanced image quality and potentially reduced radiation exposure for the object under examination. This arrangement may additionally help to boost the efficiency of the X-ray unit by enabling optimum utilization of the generated X-rays.

The specified angle of between 0 and 30°, in particular 6°, between the anode axis and the aperture plane may in particular be advantageous when using the X-ray unit in a mammography examination. Mammography is an imaging method for examining the female breast using X-rays. It may enable early identification of breast cancer and other changes in breast tissue. During mammography, the specified angle, in particular the 6-degree-angle, between the anode axis and the aperture plane may enable more uniform distribution of X-ray intensity, in particular on a side close to the thoracic wall, a reduced distance between the focal spot and the thoracic wall of the object under examination, and an improved beam geometry of the X-rays. The thoracic wall relates to a region of the object under examination where the breast adjoins the thoracic cage. Advantageously, the breast of the object under examination may be mapped as completely as possible up to the thoracic wall in the context of a mammography method. The inclination of the anode axis relative to the aperture plane by the specified angle of 6° makes it possible to reduce the distance between the focal spot and the thoracic wall by around 10%. This may enable better mapping of tissue close to the thoracic wall. The improved beam geometry may furthermore boost detail detectability.

In a further advantageous embodiment of the proposed X-ray unit, the coil may have a coil geometry that is adapted to a geometry of the X-ray tube, in particular of the vacuum housing.

The coil geometry of the coil may comprise, in particular characterize, a spatial arrangement, size, shape, number of turns, in particular coil turns, diameter, length and/or shape of a coil cross-section of the coil. The geometry of the X-ray tube may comprise, in particular characterize an external shape and/or size of the X-ray tube, in particular of the vacuum housing.

The coil geometry may advantageously be adapted to the geometry of the X-ray tube, in particular of the vacuum housing, such that the coil has an, in particular curved and/or arched, shape that follows the shape and/or outer contour of the X-ray tube, in particular of the vacuum housing. The coil may, for example, have a cylindrical shape if the X-ray tube, in particular the vacuum housing, has a cylindrical shape in the region where the coil is arranged. On the other hand, the size of the coil may be adapted to the size of the vacuum housing. The coil may advantageously be dimensioned such that it encloses the X-ray tube, in particular the vacuum housing, as closely as possible in a predefined spatial region without impairing operation of the X-ray tube.

Adaptation of the coil geometry may furthermore also comprise the positioning of the coil relative to the X-ray tube, in particular the vacuum housing. The coil may advantageously be positioned relative to the X-ray tube, in particular the vacuum housing, in such a way as to enable maximally effective magnetic field generation. This may mean, for example, that the coil is arranged concentrically around a specific region of the X-ray tube, in particular of the vacuum housing.

Adapting the coil geometry to the geometry of the X-ray tube, in particular of the vacuum housing, may offer various advantages. The adapted coil geometry may enable efficient generation of the magnetic field by way of the coil as the coil may be positioned closer to the region in which the magnetic field is required. This may result in a reduction in the current required and thus in a reduction of evolution of heat. In addition, an adapted coil geometry may help to make the generated magnetic field more homogeneous, which in turn may result in more precise deflection of the electron beam and thus in improved control of the deflection of the focal spot on the anode plate.

In a further advantageous embodiment of the proposed X-ray unit, the coil geometry may furthermore comprise a cross-sectional geometry of the coil. The cross-sectional geometry of the coil may be adapted, at least on an inner side of the coil, to the geometry of the vacuum housing within an opening region of the coil.

The cross-sectional geometry may relate to a shape and/or dimensions of a cross-section of the coil. The cross-section of the coil may be formed by a virtual section of a plane, in particular of a cross-sectional plane, through an axis, in particular an axis of symmetry, of the coil. In the case of a symmetrical, in particular rotationally symmetrical, configuration of the coil, the axis may constitute the axis of symmetry of the coil. Alternatively or additionally, the axis may be arranged along a normal direction to an opening region, in particular a plane of the opening region, of the coil. The cross-sectional plane of the coil may thus run perpendicular to the plane of the opening region of the coil. The cross-sectional geometry of the coil may for example be circular, rectangular, polygonal, trapezoidal, square and/or oval.

The inner side of the coil may denote a side of the coil facing the vacuum housing. The opening region of the coil may comprise a spatial region within the coil turns through which the vacuum housing protrudes. The opening region of the coil may comprise the spatial region in which the coil surrounds the vacuum housing.

The cross-sectional geometry of the coil on the inner side of the coil may be adapted to the geometry of the vacuum housing within the opening region of the coil by various measures. For example, the shape of the coil turns, in particular windings, may be adapted to the external shape of the vacuum housing. The cross-sectional geometry of the coil may furthermore be adapted by various manufacturing methods. The coil may, for example, have been wound, formed and/or cast, in order to achieve the desired cross-sectional geometry. Alternatively or additionally, the coil may be assembled from multiple segments, the shape of each of which is adapted, in particular in places, to the geometry of the vacuum housing. Alternatively or additionally, the cross-sectional geometry may be adapted by a suitable arrangement and orientation of the coil relative to the vacuum housing. The number and/or arrangement of the coil turns may furthermore be adapted.

Adapting the cross-sectional geometry to the geometry of the vacuum housing may enable an improved, in particular homogeneous, magnetic field distribution in the region of the electron beam. For example, the inner side of the coil may have an, in particular curved, shape that corresponds to an outer shape, in particular an outer contour, of the vacuum housing.

Adaptation of the cross-sectional geometry may enable the smallest possible distance between the coil and the vacuum housing. This may enable an increased magnetic field strength in the region of the electron beam. In addition, an adapted cross-sectional geometry may bring about a more uniform distribution of the magnetic field. This may achieve more precise deflection of the electron beam as the magnetic field is more homogeneous in the relevant region.

In a further advantageous embodiment of the proposed X-ray unit, the magnetic field may be configured to deflect the electron beam such that the focal spot on the anode plate may be displaceable by up to 2.5 mm relative to an, in particular undisplaced, zero position.

The electron beam may be deflected by the magnetic field that can be generated by way of the coil. The magnetic field may be configured such that it may deflect the electron beam in a defined region. This defined region may enable maximum displacement of the focal spot on the anode plate of up to 2.5 mm relative to the undisplaced zero position. The focal spot may be displaced by up to 2.5 mm in different, in particular opposing, directions, for example in a plane parallel to the surface of the anode plate. For example, the focal spot may in each case be displaceable by up to 2.5 mm relative to the zero position along two opposing directions. The magnetic field may thus be configured to deflect the electron beam such that the focal spot on the anode plate is in each case displaceable by up to 2.5 mm relative to the zero position, in particular along differing, in particular opposing, directions.

The precise displacement of the focal spot on the anode plate may be controlled by the strength and/or direction of the magnetic field. The magnetic field may be generated and varied by the energization of the coil.

Displacement of the focal spot by up to 2.5 mm may offer sufficient flexibility in terms of positioning the focal spot without impairing the stability and focusing of the electron beam. The value of 2.5 mm may be taken to mean the maximum displacement, wherein smaller displacements are likewise possible.

In a further advantageous embodiment of the proposed X-ray unit, the electron beam may be arranged at least in part within an opening region of the coil when the X-ray unit is in an operating state.

The opening region of the coil may comprise a spatial region within the coil through which the magnetic field that can be generated by the coil runs. The operating state of the X-ray unit may comprise a state in which the X-ray unit is activated to generate X-rays. The electron beam being arranged at least in part within the opening region of the coil may enable the magnetic field of the coil to act effectively on the electron beam and deflect the latter.

Arranging the electron beam at least in part within the opening region of the coil may offer various advantages. On the one hand, it may enable precise deflection of the electron beam as the magnetic field may be particularly homogeneous and strong in the opening region of the coil. On the other hand, arranging the electron beam in the opening region of the coil may enable a compact construction of the X-ray unit as the coil is positioned close to the X-ray tube.

The opening region of the coil may have various geometries. The opening region may, for example, be cylindrical, conical, rectangular, elliptical, prismatic, frustoconical or cuboidal. The geometry, in particular shape, of the opening region may be adapted to the geometry of the X-ray tube, in particular of the vacuum housing, and a geometry, in particular a spatial profile and/or spatial distribution and/or expansion, of the electron beam.

Arranging the electron beam at least in part in the opening region of the coil may mean that part of the electron beam or the entire electron beam runs within the opening region. The electron beam may, for example, run completely within the opening region or only a portion of the electron beam may be arranged within the opening region.

The electron beam may be arranged in the opening region of the coil by suitably positioning and orienting the coil relative to the X-ray tube. The coil may be arranged such that its opening region at least in part encloses the interspace between cathode and anode plate.

When the X-ray unit is in the operating state, the electron beam may be emitted by the cathode and accelerated toward the anode plate. In the process, the electron beam passes through the opening region of the coil and may be deflected by the magnetic field present there. The strength and direction of deflection may be controllable by the energization of the coil.

Arranging the electron beam at least in part in the opening region of the coil enables precise and controlled beam deflection. This may be utilized to purposefully control the impingement point of the electron beam on the anode plate and so influence the position of the focal spot.

One technical advantage of this arrangement may be that effective deflection of the electron beam may be achieved with comparatively low magnetic field strengths. This may help to ensure energy-efficient operation of the X-ray unit and/or reduce thermal loading of the coil.

In a further advantageous embodiment of the proposed X-ray unit, the focal spot of the X-ray tube may be arranged within the opening region of the coil when the X-ray unit is in the operating state.

This arrangement enables the magnetic field generated by the coil to permeate the electron beam in the region in which the electron beam impinges on the anode plate. This may enable precise control of the position of the focal spot on the anode plate.

The coil may advantageously be dimensioned and positioned such that its opening region completely encloses the region between cathode and anode plate. Alternatively, the coil may be so arranged that only part of the region between cathode and anode plate is encompassed by the opening region of the coil.

This arrangement may offer various technical advantages. It may enable efficient utilization of the magnetic field generated by the coil as the magnetic field acts directly on the region in which the electron beam is to be deflected. As a result, precise control of the position of the focal spot may be achieved with relatively low coil currents.

In a further advantageous embodiment of the proposed X-ray unit, the coil may take the form of an air-core coil.

An air-core coil may take the form of a coil that has no ferromagnetic core. The air-core coil may be wound from an electrically conductive material, such as copper or aluminum for example. The turns of the air-core coil may be arranged around an air core, for example a cavity or air gap, or a core of a non-ferromagnetic material.

The air-core coil may be configured to generate a magnetic field when it has an electrical current flowing through it. An air-core coil typically has lower inductance compared with coils with a ferromagnetic core. This may be advantageous for enabling rapid changes of the magnetic field.

Configuring the coil as an air-core coil may be advantageous when it comes to avoiding saturation effects that may occur with ferromagnetic cores. This may enable precise and linear control of the generated magnetic field. Furthermore, an air-core coil may also advantageously be used at high frequencies as no eddy-current losses arise in a core material.

The air-core coil may be configured in various forms and/or geometries, for example as a cylindrical coil, toroidal coil, or flat coil. The geometry and/or shape of the air-core coil may be adapted to the respective requirements and the available installation space of the X-ray unit.

In a further advantageous embodiment of the proposed X-ray unit, the turn count of the coil may be adapted as a function of a specified maximum deflection of the electron beam.

The turn count may denote a number of turns or windings of the coil, in particular a number of turns or windings as a whole or within a specified spatial portion of the coil. The turn count of the coil may be selected such that it enables the desired maximum deflection of the electron beam. Account may here advantageously have been taken of the maximum deflection, a geometry of the X-ray tube, a field strength of the magnetic field to be generated, a spatial distance between the coil and the electron beam, a maximum current intensity that may flow through the coil, a response time of the coil, a thermal loading of the coil and/or an energy of the electron beam.

The specified maximum deflection of the electron beam may denote a maximum deflection angle or a maximum deflection distance of the electron beam on the anode plate.

Increasing the turn count of the coil, in particular at an identical current, may result in a stronger magnetic field and thus a greater possible deflection of the electron beam. An excessively high turn count may also result in increased inductance of the coil, which may extend the rise time of the magnetic field.

Reducing the turn count, in particular at an identical current, may result in a weaker magnetic field and less deflection. A lower turn count may result in a faster response time of the coil as inductance is lower. This may be advantageous for applications in which a rapid change in deflection is required.

In addition, a spatial distribution, in particular density, of the turns along the coil may be adapted to achieve improved field geometry for the desired deflection. The diameter of the individual turns may be varied for local adaptation of the field strength of the magnetic field.

Adaptation of the turn count may have been optimized by simulations and/or experimental examinations in order to achieve the desired maximum deflection while simultaneously ensuring the lowest possible energy consumption and compact construction of the coil.

Adaptation of the turn count to the specified maximum deflection may enable efficient and precise control of the electron beam. This may help to improve image quality and flexibility of the X-ray machine.

In a further advantageous embodiment of the proposed X-ray unit, the coil and electron beam may be isolated from one another by a vacuum housing wall portion formed from a non-magnetic metallic material of low electrical conductivity.

The wall portion of the vacuum housing may isolate the coil and the electron beam from one another. The non-magnetic metallic material of low electrical conductivity may comprise, for example, a chromium-nickel steel alloy.

Using a non-magnetic material for the wall portion may prevent the magnetic field of the coil from being influenced or attenuated by the wall portion. This may enable effective deflection of the electron beam by the magnetic field. The low electrical conductivity of the material may reduce the formation of eddy currents in the wall portion. Eddy currents could otherwise result in unwanted heating of the wall portion and/or influence the magnetic field.

The wall portion may be configured such that it separates the interspace between the cathode and the anode plate from the region of the coil. In particular, the wall portion of the vacuum housing may be arranged between the coil and the electron beam.

The thickness of the wall portion may be selected such that, on the one hand, sufficient mechanical stability is achieved while, on the other hand, any influence on the magnetic field is minimized. The thickness of the wall portion may be in the range from 0.5 mm to 5 mm.

The wall portion may have a cylindrical or slightly conical shape that is adapted to the geometry of the vacuum housing. The axial extent of the wall portion may be selected such that it covers the entire region in which the electron beam is to be deflected.

In a further advantageous embodiment of the proposed X-ray unit, the wall portion of the vacuum housing may comprise a chromium-nickel steel alloy.

The wall portion of the vacuum housing that separates the coil and the electron beam may advantageously be formed from a material comprising a chromium-nickel steel alloy, in particular from a chromium-nickel steel alloy. The chromium-nickel steel alloy may comprise an alloy of iron, chromium and nickel, wherein the chromium content may typically be between 10% and 30% and the nickel content between 8% and 35%. Chromium-nickel steel alloys may advantageously be suitable for use in X-ray tubes due to their corrosion resistance, heat resistance, and non-magnetic properties. Examples of chromium-nickel steel alloys comprise stainless steels of the 300 series, such as 304, 316 or 321.

Using a chromium-nickel steel alloy for the wall portion of the vacuum housing may be advantageous because this alloy may have low electrical conductivity and simultaneously be non-magnetic, whereby interactions with the magnetic field of the coil may be minimized.

In a further advantageous embodiment of the proposed X-ray unit, the chromium-nickel steel alloy may take the form of an austenitic steel alloy.

An austenitic steel alloy is a specific type of stainless steel that is distinguished by its particular crystal structure. This alloy may include a large proportion of chromium and nickel. The austenitic structure may be achieved by an elevated nickel content in the alloy. Typical compositions for austenitic chromium-nickel steels may comprise for example 18% chromium and 8% nickel (18/8 steel) or 18% chromium and 10% nickel (18/10 steel). Further alloy elements such as molybdenum, titanium or niobium may be added to improve specific properties.

Austenitic steel alloys may advantageously have elevated corrosion resistance, good conformability, low magnetic permeability, and good weldability. Austenitic steel alloys may furthermore have low electrical conductivity and be non-magnetic at room temperature. Examples of austenitic steel alloys include 1.4301 (AISI 304) or 1.4401 (AISI 316).

Using an austenitic chromium-nickel steel alloy for the wall portion of the vacuum housing may offer a number of advantages. The low magnetic permeability may minimize any influence on the magnetic field of the coil. The elevated corrosion resistance may improve the durability of the X-ray tube. The good conformability may enable the production of more complex housing shapes.

The austenitic structure may furthermore also remain stable at the relatively high temperatures that may arise in X-ray tubes. This may help to maintain the mechanical and magnetic properties of the wall portion during operation of the X-ray tube.

Using an austenitic chromium-nickel steel alloy for the wall portion of the vacuum housing may thus offer an advantageous combination of mechanical, thermal, and magnetic properties that may be conducive to the functioning and performance of the X-ray unit.

In a further advantageous embodiment of the proposed X-ray unit, the cathode may have at least one emitter that may be configured to emit the electron beam.

The at least one emitter, in particular also denoted electron emitter, may for example take the form of a flat emitter, hot cathode, field-emission cathode, or photocathode. Where the at least one emitter takes the form of a hot cathode, the at least one emitter may be excited to emit electrons by heating. Where the at least one emitter takes the form of a field-emission cathode, the at least one emitter may be excited to emit electrons by application of a strong electric field. Where the at least one emitter takes the form of a photocathode, the at least one emitter may be excited to emit electrons by irradiation with light.

The at least one emitter may be configured to emit the electron beam. In particular, the at least one emitter may be configured to emit electrons and so generate the electron beam on application of a suitable voltage and/or energization. The electron beam may be emitted by thermal emission, field emission, or a combination of the two.

The emitter may be formed from a suitable emitter material. The emitter material used may be, for example, tungsten, a tungsten alloy, lanthanum boride, lanthanum hexaboride, barium oxide, cerium boride, or other suitable materials.

The cathode may comprise one or more emitters. In the case of multiple emitters, these may be identically or differently configured. The cathode may, for example, have two differently configured emitters, each of which may be configured to emit an electron beam.

In a further advantageous embodiment of the proposed X-ray unit, the at least one emitter may take the form of a flat emitter that has a slotted emitter plate for emitting the electron beam.

The at least one flat emitter may take the form of a flat electrode that may emit electrons. The at least one flat emitter may have the slotted emitter plate. The slotted emitter plate may comprise a flat and/or thin metal plate with one or more slots and/or one or more openings. The at least one slot and/or the at least one opening may enable improvement and/or control of the emission of electrons. The slots and/or openings in the emitter plate may be arranged in various patterns, in order to achieve a predefined beam shape and/or intensity of the electron beam. The emitter plate may be formed, for example, from tungsten, lanthanum boride, or a tungsten alloy. The at least one flat emitter may furthermore be configured as a dispenser cathode emitter, in particular comprising lanthanum hexaboride or barium oxide, or a field-effect emitter, in particular comprising carbon nanotubes.

The electron beam may be emitted from the at least one flat emitter by thermal emission, field emission, or a combination of the two. In thermal emission, the electrons are liberated by heating the emitter plate. In field emission, the electrons are extracted from the material of the emitter plate by a strong electric field.

The at least one flat emitter may be configured to generate a flat, tape-shaped electron beam. This may be advantageous for achieving uniform loading of the anode plate.

The cathode with the flat emitter may operate in conjunction with other components of the X-ray tube, such as for example a focusing head, in order to direct the electron beam precisely onto the anode plate. The slots and/or openings in the emitter plate may be configured to preshape and/or focus the electron beam before it impinges on further focusing elements.

Using a flat emitter of a slotted emitter plate may offer a number of technical advantages. On the one hand, a more uniform distribution of electrons in the beam may be achieved, which may result in improved image quality. On the other hand, the flat geometry of the flat emitter may help to achieve a more compact construction of the cathode and thus of the entire X-ray tube. Furthermore, the slotted design may enable better heat dissipation, which may extend the service life of the flat emitter and improve the stability of the electron beam over extended periods of operation.

In a further advantageous embodiment of the proposed X-ray unit, the cathode may have two differently configured emitters, each of which is configured to emit an electron beam.

The two emitters may have different geometries, sizes, and/or emission characteristics in order to generate differing electron beam profiles, in particular differing focal spot sizes, on the anode plate. For example, a first emitter may be configured for a focused, high-intensity electron beam, while a second emitter may be configured for a wider, lower-intensity electron beam. A first emitter may, for example, be configured for a smaller focal spot and a second emitter for a larger focal spot. This allows focal spot size to be adapted to differing imaging requirements.

The two emitters may be arranged in the cathode of the X-ray tube. They may be positioned next to one another and/or at a defined distance from and/or in a predefined orientation to one another.

Each of the two emitters may be individually drivable in order to enable selective emission of electron beams. This may enable flexible control of X-ray beam characteristics, for example by its being possible to switch between differing focal spot sizes and/or shapes. Using two differently configured emitters may advantageously enable adaptation of the X-ray beam characteristics to differing imaging requirements. For example, a focused electron beam may be used for high-resolution captures, while a wider electron beam may be advantageous for overview captures.

In a further advantageous embodiment of the proposed X-ray unit, the cathode may have a focusing head for focusing the electron beam.

The focusing head may comprise electrostatic and/or electromagnetic focusing elements, in particular configured for electrostatic and/or electromagnetic focusing of the electron beam. Focusing the electron beam may comprise bundling the electron beam. Focusing may make it possible to adapt, in particular reduce, the cross-section of the electron beam. Reducing the cross-section of the electron beam may, for example, result in a higher current density of the electron beam in the focal spot on the anode plate. This may result in improved spatial resolution of the generated X-rays.

The focusing head may comprise one or more electrodes, in particular as focusing elements. The electrodes may be configured, for example, as flat, plate, and/or ring electrodes. By applying respective, in particular different, electrical potentials to the at least one electrode, it is possible to generate an electrostatic field which is configured for focusing the electron beam. According to one embodiment, the electrodes may be maintained at the same electrical potential, in particular the same electrical potential as the cathode, in particular of the at least one emitter, when the X-ray unit is in an operating state.

Alternatively or additionally, the focusing head may comprise one or more coils. The coils may be configured to generate a magnetic field which is configured for focusing the electron beam.

The shape of the focusing head may help to focus the electron beam. The focusing head may, for example, have a conical or funnel-shaped geometry. This geometry may influence the field lines of the electrostatic and/or electromagnetic field in such a way that the electron beam is focused.

The focusing head may be manufactured from an electrically conductive material in order to enable generation of the electrostatic field. Suitable materials comprise, for example, metals or metal alloys.

Focusing of the electron beam by way of the focusing head may be performed in combination with magnetic deflection by the coil. The focusing head may prefocus the electron beam, while the coil enables precise positioning of the focal spot on the anode plate.

The focusing head may be configured as an integral component of the cathode or be arranged as a separate component in the vicinity of the cathode.

By using a focusing head to focus the electron beam, it is possible to reduce the size of the focal spot on the anode plate, which may result in improved spatial resolution of the generated X-rays.

In a further advantageous embodiment of the proposed X-ray unit, the focusing head may be of one-piece construction.

A focusing head of one-piece construction may be configured as an individual, contiguous part without separate components. The focusing head of one-piece construction may be manufactured from a single workpiece. The focusing head may, for example, have been milled, turned, forged, or otherwise machined from a metal block. Alternatively or additionally, the focusing head may be produced by additive manufacturing methods such as 3D printing.

The one-piece configuration of the focusing head may offer various advantages. On the one hand, it may result in greater mechanical stability as no junction points or joints are present that could constitute potential weak points. On the other hand, one-piece construction may simplify production and reduce costs as fewer individual parts need to be manufactured and assembled. A one-piece focusing head may furthermore enable more precise shaping, which may be advantageous for focusing the electron beam.

The focusing head of one-piece construction may be of various shapes that are optimized for focusing the electron beam. The focusing head may, for example, have a conical, concave, parabolic, or hyperbolic inner contour.

The one-piece configuration may ensure that the individual focusing elements of the focusing head are precisely oriented relative to one another. Improved focusing of the electron beam may be achieved as a result. In addition, heat dissipation within the one-piece focusing head may be improved, which may result in greater thermal stability.

The one-piece focusing head may have various integrated structures, such as for example attachment elements and/or electrical contacts. These may be directly incorporated into the workpiece without requiring separate parts.

The one-piece configuration may also improve the electrical conductivity of the focusing head as there are no transitions or junction points between individual components. This may result in a more uniform distribution of the electric field and thus in more precise focusing of the electron beam.

The focusing head may have greater mechanical stability due to the one-piece configuration. This may in particular be advantageous in the event of elevated thermal or mechanical loads, as may occur in an X-ray tube.

In a further advantageous embodiment of the proposed X-ray unit, the focusing head may comprise a chromium-nickel steel alloy or a molybdenum alloy.

A chromium-nickel steel alloy may take the form of a material that has chromium, nickel, and steel as its main constituents. The focusing head may advantageously be formed, in particular produced, at least in part, in particular completely, from a chromium-nickel steel alloy. In particular, the focusing head may consist at least in part, in particular completely, of a chromium-nickel steel alloy. The chromium-nickel steel alloy may have advantageous properties such as elevated corrosion resistance, good mechanical strength, and/or thermal stability.

The chromium-nickel steel alloy of the focusing head may, for example, take the form of austenitic stainless steel. Examples of austenitic chromium-nickel steel alloys that may be used for the focusing head comprise 1.4301 (X5CrNi18-10) or 1.4401(X5CrNiMo17-12-2).

A molybdenum alloy may take the form of a metal alloy in which molybdenum is the main alloy element. Molybdenum alloys may be distinguished by elevated strength, good thermal conductivity, and/or creep resistance at high temperatures. Examples of molybdenum alloys may comprise titanium-zirconium-molybdenum (TZM), molybdenum-hafnium-carbon (MHC) or molybdenum-lanthanum oxide (MLR).

Using a chromium-nickel steel alloy or a molybdenum alloy for the focusing head may be advantageous because these alloys exhibit good thermal conductivity. This may result in improved heat dissipation and stabler focusing of the electron beam. The elevated thermal stability of these alloys may be advantageous because the focusing head may be exposed to elevated temperatures during X-ray tube operation. Thermal stability may help to ensure that the focusing head maintains its shape and function even over an extended period of operation.

The chromium-nickel steel alloy or the molybdenum alloy of the focusing head may furthermore in each case exhibit elevated resistance to electron bombardment. This may advantageously extend the service life of the focusing head and/or reduce the maintenance intervals of the X-ray unit.

The chromium-nickel steel alloy or the molybdenum alloy of the focusing head may furthermore be configured such they exhibit slight secondary electron emission. This may result in improved focusing quality of the electron beam. In addition, the chromium-nickel steel alloy or the molybdenum alloy of the focusing head may in each case have good workability, which may enable the production of precise focusing head geometries. This may help to improve focusing of the electron beam and thus ensure higher image quality of the X-ray unit.

Using a chromium-nickel steel alloy or a molybdenum alloy for the focusing head may thus help to improve the overall performance, reliability, and durability of the X-ray unit.

In a further advantageous embodiment of the proposed X-ray unit, the focusing head may be configured for focusing in each case one or both of the electron beams that can be emitted by the two emitters.

Focusing may be performed electrostatically and/or electromagnetically. In the case of electrostatic focusing, the focusing head may comprise, for example, one or more electrodes that are maintained at a specific electrical potential. By shaping and/or arranging these electrodes, it is possible to shape an electric field such that it focuses the respective electron beam.

In the case of electromagnetic focusing, the X-ray unit may comprise one or more further coils that are configured to generate a further magnetic field. The at least one further coil may be arranged outside the vacuum housing. The further magnetic field may be guided to the focusing head by way of a yoke of the at least one further coil. This magnetic field may be shaped such that it focuses the electron beam.

The focusing head may also be configured to focus the electron beam electrostatically and electromagnetically, in particular simultaneously. This may enable particularly precise control of the electron beam.

Focusing may be separately adjustable for each of the two electron beams. This may advantageously enable focusing to be adapted to the respective requirements of X-ray unit operation. The focusing head may, for example, focus one electron beam strongly for a small focal spot, while the focusing head focuses the other electron beam less strongly for a larger focal spot.

The focusing head may furthermore be configured to focus both electron beams simultaneously. This may be advantageous for operating modes in which both emitters are used simultaneously.

The focusing head may be configured for focusing in each case one or both of the electron beams that can be emitted by the two emitters. The cathode may have two differently configured emitters, each of which may be configured to emit an electron beam. The focusing head may be configured to focus the electron beams emitted by the emitters.

The focusing head may be configured such that it may focus the electron beams from both emitters simultaneously or alternately. This may be achieved by an appropriate design of focusing head geometry and/or by dynamic adaptation of the electrical potential of the focusing head. Focusing of the electron beams by the focusing head may be controllable by a control unit. The control unit may be configured to adapt the electrical potential of the focusing head in order to adapt the focusing.

Configuring the focusing head to focus both electron beams may enable compact cathode construction since just one focusing element is required for both electron beams. This may reduce the space requirement within the X-ray tube and simplify production.

Thanks to the possibility of focusing one or both electron beams, the focusing head may enable great flexibility in the control of the X-ray tube. This may result in improved image quality and more efficient utilization of the X-ray tube in differing application scenarios.

In a further advantageous embodiment of the proposed X-ray unit, the focusing head may be configured at least by its shape for electrostatic focusing of the electron beam.

The focusing head may be configured for electrostatically focusing the electron beam. Electrostatic focusing may be achieved by the shape of the focusing head. The focusing head may, for example, have a concave shape in order to bundle the electron beam. Alternatively or additionally, the focusing head may comprise electrodes to which an electrical potential may be applied in order to generate an electrostatic field for focusing the electron beam. In particular, the electrodes may be maintained at the same electrical potential, in particular the same electrical potential as the cathode, in particular of the at least one emitter, when the X-ray unit is in an operating state. In the case of a one-piece configuration of the focusing head, the focusing head may for example be maintained at the same electrical potential as the cathode, in particular of the at least one emitter, when the X-ray unit is in the operating state.

The shape of the focusing head may be specifically adapted to the desired focusing of the electron beam. The focusing head may, for example, have an asymmetric shape in order to achieve astigmatic focusing. Alternatively, the focusing head may have a symmetrical shape in order to achieve symmetrical focusing. The shape of the focusing head may furthermore be configured to minimize unwanted electron beam scattering effects. The shape of the focusing head may be adapted to the specific geometry of the X-ray tube and the desired focusing characteristics. For example, the focusing head may be optimized for different focal spot sizes or electron beam energies.

The focusing head may be produced from an electrically conductive material in order to enable electrostatic focusing. Suitable materials comprise for example metals such as tungsten, molybdenum, a molybdenum alloy, and/or a chromium-nickel steel alloy. A surface of the focusing head may additionally be coated in order to reduce secondary electron emission.

In one embodiment, the focusing head may have two flat focusing elements, in particular focusing plates that are arranged opposite one another. The focusing elements may be maintained at the same electrical potential, in particular the same electrical potential as the cathode, in particular of the at least one emitter, when the X-ray unit is in an operating state.

Due to the identical electrical potential of the focusing plates and the cathode, an, in particular homogeneous, electric field may be generable in the interspace between the cathode and the anode plate. This electric field may enable bundling of the electron beam and a reduction in electron beam divergence. It may be possible to influence an electric field strength between the focusing elements by the distance between the focusing elements.

This configuration of the focusing head may enable effective focusing of the electron beam, in particular without additional voltage sources or complex electrode geometries. The symmetrical arrangement may help to minimize electron beam aberration and achieve uniform focusing over the entire beam cross-section.

Electrostatic focusing by the focusing head may be advantageous because it manages without additional magnetic fields and may thus be implemented more compactly and energy-efficiently than electromagnetic focusing. In addition, electrostatic focusing may be more rapidly adaptable, which enables dynamic control of the electron beam.

Electrostatic focusing by the focusing head may help to concentrate the electron beam on a small focal spot on the anode plate. This may improve the resolution and image quality of image data acquirable by way of the generated X-rays, in particular X-ray image data.

In a further advantageous embodiment of the proposed X-ray unit, the vacuum housing may have, on a cathode-side wall, an at least partially recessed pump capillary for generating a vacuum in an inner region of the vacuum housing.

The pump capillary may take the form of a tubular structure that enables a connection between an inner region of the vacuum housing and an outer region of the vacuum housing. The pump capillary may be configured to connect a vacuum pump to the inner region of the vacuum housing in order to enable evacuation of, in particular vacuum generation in, the inner region. Once evacuation is complete, the pump capillary may be closable, for example by fusion, clamping, and/or by way of a closure element.

The at least partially recessed pump capillary may be configured such that it is partially or completely let into the cathode-side wall of the vacuum housing. In this way, the overall dimensions of the X-ray tube may advantageously be reduced. In addition, the recessed pump capillary may be better protected from mechanical damage.

The cathode-side wall of the vacuum housing may denote a wall of the vacuum housing which is closest to the cathode and/or to which the cathode is attached. Arranging the pump capillary on the cathode-side wall may be advantageous because more space is typically available there than at other places on the vacuum housing. In addition, efficient evacuation of the entire inner region of the vacuum housing may be enabled by arranging the pump capillary on the cathode-side wall of the vacuum housing.

In a further advantageous embodiment of the proposed X-ray unit, the rotatable bearing mount of the anode plate may comprise an oscillation compensation.

The oscillation compensation may be configured to reduce or eliminate unwanted oscillation and/or vibration of the anode plate during rotation of the anode plate. The oscillation compensation may comprise, for example, mechanical dampers, active oscillation damping systems, and/or special bearings.

Mechanical dampers may comprise, for example, springs and/or rubber bearings. A mechanical damper may, moreover, take the form of viscous damping in which a fluid is arranged between moving parts of the bearing mount. The fluid may be configured to convert oscillatory energy into heat and so dissipate it. These mechanical dampers may be configured to absorb mechanical oscillations and to reduce any transfer of oscillations to other parts of the X-ray tube.

An active oscillation damping system may comprise sensors that acquire the oscillations of the anode plate. On the basis of the acquired oscillations, the oscillation damping system may generate counterforces to compensate the oscillations. The counterforces may be generable, for example, by electromagnetic actuators and/or piezoelectric actuators.

Special bearing-mounting systems for oscillation compensation may comprise, for example, magnetic bearings. Magnetic bearings may hold the anode plate contactlessly in suspension and so reduce mechanical friction and associated oscillation.

Oscillation compensation may help to improve the precision and stability of the X-ray beam by minimizing unwanted movement of the focal spot on the anode plate. This may result in improved image quality in X-ray imaging.

In a further advantageous embodiment of the proposed X-ray unit, the X-ray tube may furthermore comprise a drive unit for motor-driven rotation of the anode plate.

The drive unit may take the form of a component that is configured to enable and maintain rotational movement of the anode plate. The drive unit may, for example, comprise an electric motor that is mechanically connected to the anode plate. Alternatively or additionally, the drive unit may also comprise a hydraulic and/or pneumatic drive. The drive unit may furthermore comprise a control unit that is configured to control the speed and direction of rotation of the anode plate.

The motor-driven rotation of the anode plate may also take the form of a rotational movement, driven by a motor, of the anode plate about the anode axis. The motor-driven rotation may enable controlled and uniform rotation of the anode plate, which may be advantageous for a uniform distribution of the thermal loading on the anode surface. The rotational speed may be adapted to the respective operating conditions of the X-ray tube.

The oscillation compensation may interact with a rotational controller of the anode plate. For example, the rotational speed may be adapted on the basis of the acquired oscillation in order to avoid resonance.

A second aspect of one or more example embodiments of the present invention relates to a method for operating a proposed X-ray unit. In a first step, a high voltage is provided between the cathode and the anode plate. In a further step, the cathode is energized in order to provide the electron beam. In a further step, the anode plate is rotated at a rotational frequency within a specified rotational frequency range. In a further step, the coil is energized with a deflection current in order to provide the magnetic field. In a further step, the deflection of the electron beam is adapted by adapting the energization of the coil. The above-described steps may advantageously be carried out at least in part, in particular completely, simultaneously or sequentially. In addition, the order in which the above-described steps are carried out may be varied, for example the anode plate may be set in rotation before the cathode is energized.

The advantages of the proposed method substantially correspond to the advantages of the proposed X-ray unit. Features, advantages or alternative embodiments mentioned in this connection are likewise also applicable to the other claimed subjects and vice versa.

Provision of the high voltage between the cathode and the anode plate may involve applying an electrical voltage between the cathode and the anode plate. The high voltage may be provided, for example, by way of a high-voltage unit. The high voltage may advantageously be in the range from 10 kV to 300 kV.

Energization of the cathode to provide the electron beam may involve applying an electrical heating current to the cathode. The heating current may be provided, for example, by way of a power supply unit. The current intensity of the heating current applied to the cathode may advantageously be in a range from 10 mA to 20000 mA.

Rotation of the anode plate at a rotational frequency within a specified rotational frequency range may involve driving the anode plate by way of a drive unit. The anode plate may rotate about the anode axis. The drive unit may comprise, for example, an electric motor. The specified rotational frequency range may advantageously comprise rotational frequencies of at least 50 Hz, in particular between 130 Hz to 160 Hz.

Energization of the coil with the deflection current to provide the magnetic field may involve applying an electrical current to the coil. The deflection current may be provided, for example, by way of a power supply unit. The current intensity of the deflection current applied to the coil may advantageously be in a range from 0 A to 20 A.

Adaptation of the deflection of the electron beam by adapting the energization of the coil may involve modifying the deflection current, in particular a current intensity of the deflection current. The energization of the coil may be adapted, for example, by way of a control unit. The control unit may be configured to adapt the deflection current as a function of various parameters, such as for example the high voltage, the speed of movement of the X-ray unit, the direction of movement of the X-ray unit, and/or the positioning of the X-ray unit.

Provision of the high voltage between the cathode and the anode plate may generate a strong electric field within the X-ray tube, in particular in the interspace between cathode and anode plate. This electric field may be configured to accelerate the electrons emitted by the cathode.

Energization of the cathode with the heating current may result in the thermal emission of electrons. The heating current may adjust the cathode to an elevated temperature, whereby electrons may escape from the cathode material. These liberated electrons form the electron beam.

The strong electric field that is generated by the high voltage may accelerate the electrons emitted by the cathode toward the anode plate. The kinetic energy of the electrons may rise proportionally to the applied voltage.

Rotation of the anode plate at the defined rotational frequency may enable the thermal load to be distributed over a larger area of the anode plate. Rotation of the anode plate may advantageously prevent the impingement point of the electron beam, in particular the focal spot, from heating up excessively. This may advantageously extend the service life of the anode plate and enable higher performance.

Energization of the coil may generate a magnetic field that permeates the interspace between cathode and anode plate. This magnetic field may be configured to deflect the electron beam and thus influence the position of the focal spot on the anode plate. The deflection of the electron beam may be precisely controlled by adapting the energization of the coil. This may enable dynamic control of the position of the focal spot, which may be advantageous for various imaging techniques, for example tomosynthesis.

When the accelerated electrons impinge on the anode plate, they may be abruptly decelerated. A proportion of the electrons'kinetic energy may be converted into X-rays during this process.

The generated X-rays may exit from the X-ray tube through the beam exit window. The provided X-rays of the X-ray unit may furthermore be used for imaging.

The provided high voltage may influence both the energy of the electrons and the intensity and spectrum of the generated X-rays. The rotational frequency of the anode plate may advantageously be coordinated with the power of the electron beam in order to ensure optimum heat distribution. Deflection of the electron beam by the magnetic field may be precisely coordinated with the high voltage, the speed of movement of the X-ray unit, the direction of movement of the X-ray unit, and/or the positioning of the X-ray unit in order to reach a desired position of the focal spot.

Careful coordination and control of these parameters may ensure efficient and precise X-ray generation that enables elevated image quality.

In a further advantageous embodiment of the proposed method for operating an X-ray unit, the cathode may have at least one emitter and a focusing head for focusing the electron beam. Energization of the cathode may involve energizing the at least one emitter. The method may furthermore involve providing an electrical potential of the at least one emitter or a further electrical potential to the focusing head to focus the electron beam.

Energization of the at least one emitter may involve applying the electrical heating current to the at least one emitter, in particular an emitter plate of the at least one emitter. This may enable emission of electrons from the emitter plate and formation of the electron beam.

Provision of the electrical potential of the at least one emitter or a further electrical potential to the focusing head may involve applying electrical voltages to the corresponding components. Provision of the respective electrical potential may be achieved by a voltage source and corresponding electrical connections.

According to a first embodiment, the electrical potential of the at least one emitter and the electrical potential of the focusing head may be identical. In particular, a single electrical potential may be applied both to the at least one emitter and to the focusing head. This may advantageously reduce the complexity of the system and enable simpler control. Focusing of the electron beam may primarily be achieved in this embodiment by the geometric shape of the focusing head.

According to a second embodiment, different electrical potentials may be applied to the at least one emitter and the focusing head. This embodiment may enable flexible control of the electron beam. Focusing of the electron beam may be precisely controlled by adapting a potential difference between the at least one emitter and the focusing head, in particular a difference between the potential of the at least one emitter and the further potential. This may be particularly advantageous for dynamically adapting the size and/or shape of the focal spot to differing imaging requirements.

The electrical potential of the at least one emitter may take the form of an electrical voltage that is applied to the at least one emitter. This potential may influence the emission of electrons from the at least one emitter. The further electrical potential may take the form of an additional or separate electrical voltage that is applied to the focusing head. This further potential may serve to control focusing of the electron beam.

Focusing the electron beam may involve modifying, in particular reducing, a cross-section of the electron beam and/or concentrating the electron beam on a specific point or region. The focusing head may be configured for focusing the electron beam. The focusing head may for example have a special geometry and/or electrical properties for focusing the electron beam.

Improved focusing and control of the electron beam may be achieved by the combination of emitter, focusing head and targeted use of electrical potentials. This may result in higher image quality and more efficient utilization of the X-rays.

In a further advantageous embodiment of the proposed method for operating an X-ray unit, the cathode may have two differently configured emitters, each of which may be configured to emit an electron beam. The emitters may be selectively or simultaneously energized.

Selective energization of the emitters may involve energizing in each case one of the two emitters. Selective energization of the emitters may in particular involve just one of the emitters being energized at a specific point in time. Just one of the emitters may advantageously be energized in each case in order to generate an electron beam with specific characteristics. Different focal spot sizes and/or electron beam energies may, for example, be generable by selective energization of the emitters. The emitter respectively to be energized may be selected manually, in particular on the basis of user input, or automatically, for example on the basis of a specified measurement protocol. Energization of the emitters may be controlled by a control unit. The control unit may be configured to adapt the energization of the emitters on the basis of operating parameters, for example beam intensity, focal spot size, and/or imaging requirement.

Simultaneous energization of the emitters may involve energizing both emitters at a specific point in time. Simultaneous energization of both emitters may advantageously enable generation of an, in particular combined, electron beam with a higher intensity and/or more complex beam geometry. Characteristics of the, in particular combined, electron beam may additionally be finely tuned by adapting and/or varying the energization current intensity of the individual emitters.

The possibility of selective or simultaneous energization of the emitters may improve the flexibility and adaptability of the X-ray unit. The emitters may be selectively or simultaneously energized in combination with the deflection of the electron beam by the magnetic field of the coil. This may enable precise control of the position and/or characteristics of the focal spot on the anode plate.

This embodiment of the method may offer the advantage of enabling flexible and adaptable electron beam generation. The possibility of selecting between or combining different emitters may make it possible to adapt the method to differing imaging requirements. This may result in improved image quality and greater flexibility of the X-ray machine.

In a further advantageous embodiment of the proposed method for operating an X-ray unit, the specified rotational frequency range may comprise rotational frequencies of at least 50 Hz, in particular of 130 Hz to 160 Hz.

The rotational frequency range may define a range of rotational frequencies within which the anode plate of the X-ray unit is intended to rotate when the X-ray unit is in an operating state. The rotational frequency may indicate a number of, in particular complete, revolutions of the anode plate per second. The specified rotational frequency range of at least 50 Hz, in particular of 130 Hz to 160 Hz, may thus correspond to a rotational speed of the anode plate of at least 50, in particular of 130 to 160, revolutions per second. This range may be suitable for operation of the X-ray tube in a medical imaging device.

Specifying the rotational frequency range may enable stable and efficient operation of the X-ray tube. An excessively low rotational frequency may result in a non-uniform distribution of heat on the anode plate, while an excessively high rotational frequency may cause mechanical loads and vibration. Compliance with the specified rotational frequency range may additionally enable a uniform distribution of the focal spot on the anode plate. This may help to extend the service life of the rotating anode.

The rotational frequency of the anode plate may be controlled by a drive unit. The drive unit may, for example, comprise an electric motor that is connected to the anode plate via a shaft. The rotational frequency of the anode plate may be adjusted by closed-loop control of the rotational speed of the drive unit, in particular of the electric motor.

Reproducible image quality in repeated captures may moreover be achieved by specifying the rotational frequency range. This may be of significance in particular in medical applications in which constant image quality is required.

In a further advantageous embodiment of the proposed method for operating an X-ray unit, it may be monitored whether the instantaneous rotational frequency of the anode plate is within the specified rotational frequency range. A warning may be provided in the negative case.

Alternatively or additionally, further operating parameters of the X-ray unit may be monitored, for example a temperature and/or a current supply, and/or voltage supply, wherein, in the event of a deviation from a predefined value or range of values being identified, a further warning may be provided.

Monitoring of whether the instantaneous rotational frequency of the anode plate is within the specified rotational frequency range may involve continuously or periodically acquiring and evaluating the instantaneous rotational frequency of the anode plate. The instantaneous rotational frequency may, for example, be acquired by way of a sensor that detects the rotation of the anode plate. Alternatively or additionally, the instantaneous rotational frequency may be acquired by way of a calibrated drive unit for rotating the anode plate. Monitoring may be carried out by a control unit of the X-ray unit.

The warning may comprise a visual, acoustic, and/or tactile signal that indicates a deviation of the instantaneous rotational frequency from the specified rotational frequency range. The warning may comprise various items of information, for example the instantaneous rotational frequency, a deviation of the instantaneous rotational frequency from the specified rotational frequency range and/or a recommended action for correcting the rotational frequency. The warning, in particular a graphical representation of the warning, may be displayed, for example, via a display unit. Alternatively or additionally, the warning may be output as an acoustic signal via a loudspeaker. Alternatively or additionally, the warning may be provided as a tactile signal, for example by way of a vibration motor. The warning may advantageously make it possible to make a user aware of a possible malfunction and/or suboptimal operating conditions of the X-ray unit.

The warning may be provided automatically by the control unit of the X-ray unit when it is established that the instantaneous rotational frequency is outside the specified rotational frequency range. The control unit may be configured to provide the warning via a corresponding output interface.

Reliable and safe operation of the X-ray unit may be ensured by monitoring the rotational frequency and the provision of a warning in the event of deviations. Possible problems and/or suboptimal operating states may be identified and remedied in good time, which may improve image quality and service life of the X-ray unit.

A third aspect of one or more example embodiments of the present invention relates to a method for calibrating a proposed X-ray unit. In a first step a), an absorption mask is positioned between the beam exit window of the X-ray unit and an X-ray acquisition unit. In a further step b), a high voltage is provided between the cathode and the anode plate. In a further step c), the cathode is energized to provide the electron beam. In a further step d), the anode plate is rotated at a rotational frequency within a specified rotational frequency range. In a further step e), the coil is energized with a deflection current to provide the magnetic field. In a further step f), a position of a mapping of the absorption mask is acquired by way of the X-ray acquisition unit. Steps b) to f) are carried out repeatedly for a plurality of differing high voltages and/or deflection currents. A correlation between the deflection of the electron beam and the deflection current and/or between the deflection of the electron beam and the high voltage is identified on the basis of the plurality of differing high voltages and/or deflection currents and the associated positions of the mapping. In a further step, an item of calibration information that characterizes the correlation is provided.

The above-described steps may advantageously be carried out at least in part, in particular completely, simultaneously or sequentially. In addition, the order in which the above-described steps b) to e) are carried out may be varied, for example the anode plate may be set in rotation before the cathode is energized.

The advantages of the proposed method for calibrating an X-ray unit substantially correspond to the advantages of the proposed X-ray unit and/or of the proposed method for operating an X-ray unit. Features, advantages or alternative embodiments mentioned in this connection are likewise also applicable to the other claimed subjects and vice versa.

The absorption mask may take the form of a structure that partially absorbs the X-rays. The absorption mask may for example comprise an X-ray-absorbing material, in particular lead or a lead alloy. The absorption mask may have patterns and/or structures, in particular made of the X-ray-absorbing material, that are configured to produce a defined mapping on the X-ray acquisition unit. This mapping may be taken to be a kind of shadow of the absorption mask that is produced by the X-rays. A position and/or shape of this mapping on the X-ray acquisition unit may then be used to calibrate the X-ray unit.

Positioning the absorption mask may involve an, in particular targeted, placement and/or orientation of the absorption mask between the beam exit window of the X-ray unit and the X-ray acquisition unit. The absorption mask may be positioned manually or by automated mechanisms. Positioning the absorption mask may additionally involve a fine adjustment in various degrees of freedom, for example translation and/or rotation of the absorption mask. The absorption mask is positioned between the beam exit window of the X-ray unit and the X-ray acquisition unit, in particular in a region illuminated by the X-rays.

The X-rays that are emitted from the beam exit window may illuminate, in particular at least partially pass through, the absorption mask. Some of the X-rays may be absorbed by the absorption mask, while the remainder of the X-rays are transmitted.

Steps b) to e) of the proposed method for calibrating an X-ray unit, in particular providing the high voltage between the cathode and the anode plate, energizing the cathode to provide the electron beam, rotating the anode plate at the rotational frequency within the specified rotational frequency range, and energizing the coil with the deflection current to provide the magnetic field, may be in particular be carried out analogously to the corresponding steps of the proposed method for operating an X-ray unit.

A position of a mapping of the absorption mask may be acquired by way of the X-ray acquisition unit. Acquisition of the position may involve detecting and locating the mapping on the X-ray acquisition unit, in particular an X-ray-sensitive layer of the X-ray acquisition unit. The X-ray acquisition unit may take the form of a device that is configured to convert X-rays into an electrical signal and/or an image.

Steps b) to f) may be carried out repeatedly for a plurality of differing high voltages and/or deflection currents. Repeatedly carrying out said steps may involve multiple passes of the steps with different parameters. The plurality may comprise, for example, 5, 10, 20 or more differing combinations of high voltages and deflection currents.

Varying the high voltage and/or deflection current may have specific effects on the position of the mapping of the absorption mask on the X-ray acquisition unit.

Increasing the high voltage between cathode and anode plate may increase the velocity of the electrons in the electron beam. This may result in the electrons being less strongly deflected by the magnetic field. As a consequence, the position of the mapping of the absorption mask on the X-ray acquisition unit may be repositioned as the high voltage rises, in particular at an identical deflection current, toward a zero position, in particular a position in which the electron beam is not deflected.

The velocity of the electrons in the electron beam may be reduced by reducing the high voltage between cathode and anode plate. This may result in stronger deflection of the slower electrons by the magnetic field. Thus, a reduction in the high voltage, in particular at an identical deflection current, may reposition the mapping of the absorption mask further away from the zero position.

The energization of the cathode may advantageously be adapted as a function of the high voltage, in particular such that a power, in particular a product of high voltage and current intensity of a tube current as a consequence of the energization of the cathode, remains substantially constant, for example amounts to a power of 5 W. For example, the current intensity of cathode energization may be correspondingly reduced in the event of an increase in the high voltage between cathode and anode plate.

The tube current and cathode energization may be closely related to one another. Energization of the cathode, also denoted heating current, may involve providing an electrical current that flows through the cathode, in particular an emitter, for example an incandescent filament or an emitter plate. This heating current may heat the cathode to elevated temperatures, typically in the range from 1800 °C to 2400 °C, which may result in the thermal emission of electrons.

The tube current, in contrast, may relate to the current of electrons flowing from the cathode to the rotating anode. This current may be generated and controlled by the high voltage between cathode and anode. The quantity of electrons emitted and thus the tube current may depend directly on the temperature of the cathode, which may in turn be determined by the energization of the cathode.

There may be a nonlinear relationship between the energization of the cathode and the resultant tube current. In the event of higher energization of the cathode, the temperature may increase, which may result in increased electron emission and thus in a higher tube current. This relationship may roughly obey Richardson's law, which describes electron emission as a function of temperature.

In modern X-ray tubes, the tube current may be regulated by precise control of cathode energization. The heating current for the cathode may typically be in the range from 10 mA to 20000 mA, while the resultant tube current may vary between 0.1 mA and 1000 mA depending on application.

A change in the deflection current in the coil may directly influence the strength of the generated magnetic field. A higher deflection current may generate a stronger magnetic field, which may result in greater deflection of the electron beam. An increase in deflection current may, in particular at an identical high voltage, reposition the mapping of the absorption mask further away from the zero position. A reduction in deflection current may, in particular at an identical high voltage, reposition the mapping of the absorption mask toward the zero position.

The position of the mapping may change in differing directions depending on the orientation of the magnetic field and the geometry of the X-ray unit. Repositioning may occur in the horizontal and/or vertical direction on the X-ray acquisition unit, in particular the X-ray-sensitive layer of the X-ray acquisition unit.

A correlation between the deflection of the electron beam and the deflection current and/or between the deflection of the electron beam and the high voltage may be identified on the basis of the plurality of differing high voltages and/or deflection currents and the associated positions of the mapping. Identifying the correlation may involve analyzing and correlating the acquired data in order to establish a relationship between the parameters.

Systematically varying the high voltage and/or deflection current may make it possible to create a detailed map of the acquired positions of the mapping of the absorption mask. This map may be used as the basis for calibrating the X-ray unit and enable precise control of electron beam deflection in various operating states.

Calibration information that characterizes the correlation may be provided. Provision of the calibration information may involve generating and saving data that describes the identified correlation. The calibration information may, for example, take the form of a table, a mathematical function and/or an algorithm.

The method for calibrating an X-ray unit may offer the advantage of enabling precise control of electron beam deflection as a function of various operating parameters. Improved image quality and more precise positioning of the focal spot may be achieved as a result.

In a further advantageous embodiment of the proposed method for calibrating an X-ray unit, the X-ray acquisition unit may comprise an X-ray camera and/or an X-ray detector.

The X-ray camera may take the form of an imaging device that is configured to convert incident X-rays into visible light and to record the latter. The X-ray camera may, for example, comprise a scintillator that converts the X-rays into visible light and a photosensitive camera for capturing the visible light. Cesium iodide or gadolinium oxysulfide may, for example, be used as the scintillator. The X-ray camera may advantageously have an optical sensor, in particular a CCD or CMOS sensor, for detecting the visible light generated by the scintillator.

The X-ray camera may advantageously have a CCD sensor. When a CCD sensor is used, the X-ray camera may have elevated sensitivity and low noise. The CCD sensor may convert the light emitted by the scintillator into electrical charges which may then be read out and converted into digital signals.

The X-ray camera may alternatively have a CMOS sensor. CMOS sensors may have a faster readout speed and lower power consumption in comparison with CCD sensors. The CMOS sensor may be configured to convert the light generated by the scintillator directly into electrical signals.

The X-ray camera may additionally have an optical system between the scintillator and the sensor, in particular the CCD or CMOS sensor. This optical system may be configured to map the light pattern generated by the scintillator onto the sensor, and optionally to magnify it or scale it down. The optical system may comprise, for example, multiple lenses and/or a fiber taper. The X-ray camera may be arranged at a predefined distance from or directly adjacent to the X-ray unit, in particular the beam exit window.

Reading out the sensor, in particular the CCD or CMOS sensor, may be controlled by special electronics that enable various exposure times and/or readout modes to be set. The X-ray camera may furthermore comprise an image amplifier between scintillator and sensor that is configured to increase light yield.

The X-ray detector may be configured to convert X-rays directly into electrical signals. The X-ray detector may, for example, comprise a semiconductor detector that is configured to convert the X-rays directly into electrical charges. The semiconductor material may comprise, for example, amorphous selenium, cadmium telluride and/or silicon. The X-ray detector may alternatively comprise multiple layers. The first layer may comprise a scintillator that may be configured to convert incident X-rays into visible light. The scintillator material used may be, for example, cesium iodide or gadolinium oxysulfide. A further layer comprising a plurality of detector elements may be arranged, relative to the incident direction of the X-rays, downstream of the layer comprising the scintillator. The detector elements may in each case take the form of photodiodes. The detector elements, in particular the photodiodes, may be configured to convert the light generated by the scintillator into electrical signals. The photodiodes may, for example, be placed in a spatial matrix arrangement in order to enable flat panel detection. Readout electronics may be connected to the detector elements. The readout electronics may be configured to read out and digitize the electrical signals generated by the photodiodes. The digitized signals may then be transferred to a control unit for further processing. In particular, the X-ray detector may take the form of an integrating X-ray detector. The integrating X-ray detector may be configured to accumulate the incident X-rays over a specific period, in particular an integration period. The detector elements may in this case store the electrical charges generated by the X-rays. The entire accumulated charge may be read out at the end of the integration period.

The X-ray detector may for example take the form of a flat panel detector. The flat panel detector may comprise a plurality of detector elements, each of which may be configured to convert the incident X-rays into electrical signals.

The X-ray unit and the X-ray detector may be arranged in a defined arrangement and opposite one another. The X-ray detector may advantageously be arranged at a distance from the X-ray unit, in particular at a distance such that an object under examination can be arranged between the X-ray unit and the X-ray detector.

Deflection of the electron beam may directly influence the position of the mapping of the absorption mask. When the electron beam is deflected, the focal spot on the anode plate is repositioned, which may in turn result in repositioning of the X-rays. This repositioning of the X-rays may result in a corresponding repositioning of the mapping of the absorption mask on the X-ray acquisition unit.

The relationship between the deflection of the electron beam and the position of the mapping may be considered virtually linear. Deflecting the electron beam by a specific angle relative to a zero position, may result in a proportional repositioning of the mapping of the absorption mask. The precise relationship between the deflection of the electron beam and the position of the mapping may be determined by geometric calculations taking account of the distance between the anode plate and the X-ray acquisition unit as well as of the anode angle.

In a further advantageous embodiment of the proposed method for calibrating an X-ray unit, at least four differing high voltages and/or deflection currents may in each case be specified for repeatedly carrying out steps b) to f).

The high voltages may advantageously be specified in a range from 10 kV to 300 kV. In particular, 23 kV, 28 kV, 35 kV and 40 kV may be specified as high voltages for repeatedly carrying out steps b) to f). In this case, a constant deflection current or differing deflection currents may be specified. In particular, a constant deflection current of 10 A may be specified. Furthermore, respective current intensities of the tube current as a consequence of energizing the cathode, in particular the heating current of the cathode, may in this case be specified, for example 217 mA for a high voltage of 23 kV, 175 mA for a high voltage of 28 kV, 142 mA for a high voltage of 35 kV and 125 mA for a high voltage of 40 kV.

The deflection currents may, for example, be specified in a range from 0 A to 10 A. In this case, a constant high voltage or differing high voltages may be specified.

Specifying at least four differing high voltages and/or deflection currents may provide a sufficient number of measurement points to enable a reliable correlation between the deflection of the electron beam and the deflection current and/or between the deflection of the electron beam and the high voltage. A larger number of measurement points may further improve the accuracy of the correlation.

Steps b) to f) may be carried out repeatedly for each combination of the differing high voltages and deflection currents. Alternatively, they may be carried out repeatedly for a selection of combinations that are considered representative of the entire operating range of the X-ray unit.

The various high voltages and/or deflection currents may be automatically specified by a control unit of the X-ray unit or imaging device. Alternatively, they may be specified manually by an operator.

Using at least four differing high voltages and/or deflection currents may advantageously enable a comprehensive characterization of the behavior of the X-ray unit over a broad operating range. This may result in more accurate calibration and thus improved image quality and positioning accuracy of the focal spot.

In a further advantageous embodiment of the proposed method for calibrating an X-ray unit, identifying the correlation between the deflection of the electron beam and the deflection current and/or between the deflection of the electron beam and the high voltage of the electron beam may in each case involve an interpolation.

An interpolation may be taken to mean a mathematical method for estimating intermediate values within a series of known data points. In the context of the proposed method for calibrating an X-ray unit, interpolation may enable determination of a deflection of the electron beam for a deflection current and/or a high voltage that have not been directly measured.

A linear interpolation, in which it is assumed that the relationship between the measured data points is linear, may be used here. Alternatively, a polynomial interpolation that models a more complex relationship between the data points may be used. Further advantageous interpolation methods include a cubic spline interpolation and/or a Lagrange interpolation.

Identifying the correlation by way of interpolation may advantageously give rise to a continuous function for the relationship between the deflection of the electron beam and the deflection current and/or high voltage. This may enable more precise control of the deflection of the electron beam, including for values that are located between directly measured calibration points. A calibration point may here in each case comprise a specified deflection current or a specified high voltage and an associated deflection of the electron beam.

The interpolation result may be saved as a lookup table and/or as a mathematical function in order to enable rapid access to the correlation during operation of the X-ray unit.

The interpolated correlation may be used to determine the electron beam deflection corresponding to any desired values of the deflection current and/or high voltage. This enables precise control of deflection, including for operating states that do not exactly correspond to the points measured during calibration. Conversely, the interpolated correlation may also be used to determine the deflection current and/or the high voltage required for a desired deflection of the electron beam. This may be utilized for targeted positioning of the focal spot on the anode plate.

Using interpolation for correlation may offer a number of technical advantages. On the one hand, the number of measurement points required during calibration may be reduced, so accelerating the calibration process. On the other hand, the continuous correlation enables finer and more precise control of the deflection of the electron beam, which may result in improved image quality and more accurate positioning of the focal spot.

A fourth aspect of one or more example embodiments of the present invention relates to an imaging device. The imaging device comprises a proposed X-ray unit, an X-ray detector, and a control unit. The X-ray unit and the X-ray detector are arranged opposite one another. The X-ray detector is configured to acquire X-rays that can be emitted by the X-ray unit. The X-ray unit is bearing-mounted so as to be movable relative to the X-ray detector. The control unit is configured to adapt energization of the coil as a function of an instantaneous position of the X-ray unit in such a way that the electron beam that can be emitted by the cathode is deflected by way of the magnetic field to minimize any movement of the focal spot relative to the X-ray detector.

The advantages of the proposed imaging device substantially correspond to the advantages of the proposed X-ray unit, the proposed method for operating an X-ray unit, and/or the proposed method for calibrating an X-ray unit. Features, advantages or alternative embodiments mentioned in this connection are likewise also applicable to the other claimed subjects and vice versa.

The X-ray unit and the X-ray detector are arranged opposite one another. In this respect, the beam exit window, in particular the aperture unit, of the X-ray unit and an X-ray-sensitive layer, in particular an X-ray-sensitive surface, of the X-ray detector may advantageously be arranged facing one another. The X-ray unit and the X-ray detector may advantageously be arranged opposite one another in such a way that the X-rays that can be emitted by the X-ray unit may illuminate an X-ray-sensitive layer, in particular an X-ray-sensitive surface, of the X-ray detector, in particular after interacting with an object under examination arranged between the X-ray unit and the X-ray detector.

Moreover, the X-ray unit and the X-ray detector may be arranged in a defined arrangement relative to one another.

The X-ray detector may be configured to receive the X-rays that can be emitted by the X-ray unit and convert them into electrical signals. The X-ray detector may have a plurality of detector elements, which may be arranged in a matrix arrangement. These detector elements may be manufactured from materials such as amorphous silicon, amorphous selenium, and/or cadmium telluride that have an elevated sensitivity to X-rays. The X-ray detector may be configured, for example, as a flat panel detector and/or line detector and/or multi-line detector.

The X-ray detector may have a scintillator layer that is configured to convert the incident X-rays into visible light. Furthermore, the X-ray detector may have photosensors, for example photodiodes, that are configured to acquire the light provided by the scintillator layer and convert it into electrical signals. The scintillator layer may include cesium iodide or gadolinium oxysulfide, for example.

To ensure high sensitivity, the X-ray detector may comprise readout electronics that enable rapid and low-noise signal processing. The X-ray detector may additionally comprise a cooling system in order to reduce thermal noise and improve image quality. The cooling system may, for example, comprise Peltier elements and/or liquid cooling.

In a further embodiment, the X-ray detector may take the form of an energy-resolving X-ray detector. The energy-resolving X-ray detector may be configured to acquire not only the intensity but also the energy of the incident X-rays, in particular of the incident X-ray photons. To this end, the X-ray detector may include a semiconductor layer, in particular comprising cadmium telluride and/or cadmium zinc telluride, that is configured to convert X-rays, in particular X-ray photons, directly into electrical signals.

In a further embodiment, the X-ray detector may take the form of multilayer detector. The multilayer detector may comprise multiple detector layers that are arranged one above the other and may in each case be configured, in particular optimized, to acquire a differing energy range of the X-rays.

An upper detector layer of the multilayer detector, in particular relative to a direction of ray incidence, may comprise amorphous silicon or amorphous selenium, for example. This detector layer may be particularly sensitive to low-energy X-rays and may in particular have a thickness of around 200 to 500 micrometers. A lower detector layer, in particular relative to the direction of ray incidence, may comprise cesium iodide, for example. This detector layer may in particular have a thickness of around 200 to 500 micrometers and may be optimized for the detection of X-rays in the higher energy range.

Thin filter layers of materials such as for example copper and/or aluminum may be arranged between the individual detector layers. These filter layers may be configured to modify an energy spectrum of the X-rays for the respective detector layer located therebelow.

Use of a multilayer detector may enable improved energy resolution and an extended dynamic range compared with conventional single-layer detectors.

The X-ray detector may furthermore comprise an apparatus for reducing scattered radiation, in particular to improve the signal-to-noise ratio. The apparatus for reducing scattered radiation may comprise an anti-scatter grid. Alternatively or additionally, the apparatus for reducing scattered radiation may be configured to apply software-based scatter correction algorithms to the provided electrical signals.

The scintillator or semiconductor layer of the X-ray detector may form the X-ray-sensitive layer of the X-ray detector.

The X-ray unit is bearing-mounted so as to be movable, in particular capable of translational and/or rotational motion, relative to the X-ray detector. The bearing mount allowing translational motion may be implemented, for example, by way of a linear guide system. The linear guide system may comprise precision rails and plain bearings that enable low-friction, precise linear movement of the X-ray unit. Translational motion may be drivable by way of a motor, for example a linear motor or a spindle drive. The imaging device may furthermore comprise a position acquisition unit, for example an encoder system, that may be configured to determine the precise instantaneous position of the X-ray unit. The rotatable bearing mount may be implemented, for example, by way of a revolute joint and/or a ball bearing. The rotation may be drivable by way of a motor, for example a servomotor. The motor may in particular be connected to a pivot bearing via a gear unit. The position acquisition unit may, for example, have an angular encoder, in particular an optical and/or magnetic angular encoder, for angle acquisition.

The movable bearing mount of the X-ray unit may additionally have damping elements that are configured to reduce vibration and enable stable imaging. The damping elements may, for example, comprise hydraulic and/or pneumatic dampers.

For precise movement control, the control unit may furthermore be configured to adapt the movement of the X-ray unit, in particular in real time, and to synchronize it with the acquisition of image data, in particular X-ray image data, by the X-ray detector.

The movable bearing mount of the X-ray unit may be so designed as to enable various capture geometries, for example tomosynthesis and/or panoramic imaging of an object under examination. A movement path, in particular trajectory, of the X-ray unit relative to the X-ray detector may advantageously be adaptable to specific requirements of the respective imaging technique. In particular, the control unit may be configured to adapt the trajectory of the X-ray unit.

The movable bearing mount of the X-ray unit may, for example, enable movement of the X-ray unit in a vertical plane. In this case, the trajectory of the X-ray unit may follow a linear path. This enables adaptation of the positioning of the X-ray unit to differing objects under examination and/or patient positions.

For application in the context of tomosynthesis, the trajectory of the X-ray unit may be arcuate. In this case, the arcuate trajectory may encompass an angular range of around ±25°, in particular ±7.5°, wherein an axis of rotation of the trajectory may be arranged at a predefined distance relative to the X-ray detector, in particular at a geometric center point of an average object under examination, in particular an average breast, or may run through the X-ray detector, in particular parallel to a surface, in particular an X-ray-sensitive layer, of the X-ray detector. The X-ray unit may be movable in discrete steps or continuously.

The X-ray unit may advantageously be configured to carry out a combined movement. For example, the trajectory of the X-ray unit may comprise simultaneous vertical translation and rotation about a horizontal axis. The trajectory may in this case be of spiral or helical configuration.

The instantaneous position of the X-ray unit may describe a spatial position and/or orientation and/or posture of the X-ray unit, in particular relative to the X-ray detector. The control unit may be configured to acquire the instantaneous position of the X-ray unit, for example by way of a positioning sensor. Alternatively or additionally, the X-ray unit may be movably bearing-mounted by way of a calibrated movement unit. In this case, the calibrated movement unit may be configured to provide positioning information including information about the instantaneous position of the X-ray unit to the control unit.

The control unit is configured to adapt energization of the coil as a function of, in particular on the basis of, the instantaneous position of the X-ray unit. Adaptation of the energization of the coil as a function of the instantaneous position of the X-ray unit may involve determining and specifying, in particular adjusting, the deflection current for energization of the coil. To this end, the control unit may be configured to access stored, in particular saved, calibration information that characterizes a correlation between the deflection of the electron beam and the deflection current. The calibration information may have been provided, for example by way of a proposed method for calibrating an X-ray unit. By adapting the energization of the coil, in particular of the deflection current, it may be possible to influence a strength and/or direction of the magnetic field generated by the coil.

Minimizing the movement of the focal spot relative to the

X-ray detector may involve reducing or avoiding any relative repositioning between the focal spot and the X-ray detector. This may be advantageous in order to achieve a constant image quality during capture of image data, in particular X-ray image data, in particular in the case of capture sequences with a moving X-ray unit such as for example in the case of tomosynthesis.

The control unit may be configured to adapt energization of the coil, in particular the deflection current, as a function of relative movement between the X-ray unit and the X-ray detector, in order to achieve minimization of the movement of the focal spot. A change in the deflection current may result in a modification of the magnetic field generated by the coil, which may in turn influence the position of the electron beam and thus of the focal spot on the anode plate.

In the event of an increase in the deflection current, the magnetic field may grow in strength, which can lead to stronger deflection of the electron beam. This may bring about repositioning in the focal spot on the anode plate. Conversely, a reduction in the deflection current may lead to weaker deflection and repositioning of the focal spot in the opposing direction.

To compensate for a relative movement between the X-ray unit and the X-ray detector, the deflection current may be adaptable, in particular continuously adaptable. If, for example, the X-ray unit is moving in a given direction, the control unit may modify the deflection current in such a way that the focal spot is shifted in an opposing direction. The deflection current may here be adapted proportionally to a speed and direction of the relative movement.

In more complex movement sequences, such as may occur during tomosynthesis, dynamic adaptation of the deflection current may be necessary. In this respect, the control unit may be configured to acquire, in particular in real time, the instantaneous position of the X-ray unit and regulate the deflection current accordingly, in order to ensure a maximally constant position of the focal spot relative to the X-ray detector.

The precise adaptation of the deflection current may depend on various factors, such as the geometry of the X-ray unit, the strength of the generated magnetic field, and the energy of the electron beam. Precise calibration, for example by way of a proposed method for calibrating an X-ray unit, and use of the provided calibration information may make it possible to establish suitable energization of the coil for each position of the X-ray unit.

Thanks to this adaptive control of the deflection current, it is possible to achieve minimization of the movement of the focal spot.

In one further advantageous embodiment of the proposed imaging device, the imaging device may further comprise a stand unit, a first holding unit, and a second holding unit. The first holding unit may be arranged in a rotatably bearing-mounted manner on the stand unit. The X-ray unit may be arranged on the first holding unit. The second holding unit may be arranged on the stand unit. The X-ray detector may be arranged on the second holding unit. The first and the second holding units may hold the X-ray unit and the X-ray detector opposite one another.

The stand unit may take the form of an, in particular stationary or mobile, base or main framework of the imaging device. The stand unit may be configured to bear and/or support the other components of the imaging device. In particular, the stand unit may be configured to bear and stabilize the first and second holding units. The stand unit may take the form, for example, of a floor stand, wall mount, robot arm, or ceiling mount.

The first holding unit may take the form of an, in particular mobile, support structure, in particular an arm and/or bracket, for the X-ray unit. In particular, the first holding unit may hold, in particular bear, the X-ray unit. In this case, the X-ray source may be attached to the first holding unit. The first holding unit may be rotatably bearing-mounted on the stand unit, so as to enable movement of the X-ray unit. The rotatable bearing mount of the first holding unit on the stand unit may enable rotational movement of the X-ray unit about one or more axes. This may enable flexible positioning of the X-ray unit relative to the X-ray detector. The rotatable bearing mount may, for example, be implemented by way of a revolute joint, a pivot shaft, and/or a swivel mechanism.

The second holding unit may take the form of an, in particular fixed or mobile, support structure, in particular a column, arm, or plate, for the X-ray detector. In particular, the second holding unit may hold, in particular bear, the X-ray detector. In this case, the X-ray detector may be attached to the second holding unit. The second holding unit may be arranged on, in particular attached to, the stand unit, in order to hold the X-ray detector in a defined position relative to the stand unit. The second holding unit may take the form of an adjustable, in particular height-adjustable, platform. In addition, the second holding unit may have an inclination function, in particular an inclination about a horizontal axis, so as to enable optimal positioning of the object under examination.

As a result of the X-ray unit and the X-ray detector being held opposite one another by the first and second holding units, a defined geometric arrangement of the X-ray unit relative to the X-ray detector may be enabled. This defined arrangement may be advantageous for precise imaging. Arranging the X-ray unit on the first holding unit and the X-ray detector on the second holding unit may enable precise orientation and positioning of the two components relative to one another. The first and second holding units may hold the X-ray unit and the X-ray detector opposite one another and in a defined arrangement, in particular a defined geometric relationship, relative to one another.

The first and the second holding units may be manufactured from stable, rigid materials such as metal and/or composite materials, in order to ensure precise positioning of the X-ray unit and of the X-ray detector. The holding units may additionally be equipped with damping elements, in order to reduce vibration.

The rotatable bearing mount of the first holding unit combined with the arrangement of the second holding unit may enable various capture positions and angles, while keeping the X-ray unit and X-ray detector positioned opposite one another. This may enable flexible imaging from different perspectives.

In one embodiment, the imaging device may take the form of a mammography unit. The mammography unit may be designed specifically for examination of the female breast and enable high-resolution X-ray imaging.

The X-ray unit of the mammography unit may comprise a special X-ray tube that is optimized for the requirements of breast imaging. This X-ray tube may have an anode plate configured as a single-angle plate with an angle of 16°, in order to ensure an optimal beam geometry for breast examination. The anode plate may be manufactured from a tungsten-rhenium alloy, to enable elevated thermal resistance and efficient X-ray production.

The coil of the X-ray unit may be configured as an air-core coil and arranged outside the vacuum housing. This arrangement may enable precise control of the magnetic field without impairing the vacuum integrity of the X-ray tube. The coil may be dimensioned such that it allows deflection of the electron beam by up to 2.5 mm relative to a zero position on the anode plate, so allowing flexible adaptation of the focal spot.

The X-ray detector of the mammography unit may take the form of a digital flat panel detector, which may exhibit an elevated spatial resolution of for example 50 to 100 micrometers per pixel.

The first holding unit, to which the X-ray unit is attached, may be L- or J-shaped. The first holding unit may enable rotation of the X-ray unit about the breast of the object under examination. This may enable tomosynthesis captures of the breast to be examined, for example. The X-ray unit may rotate through an angular range of ±25°, wherein the control unit adapts energization of the coil, in particular continuously, so as to optimize the position of the focal spot relative to the X-ray detector.

The second holding unit, to which the X-ray detector is attached, may take the form of an adjustable platform. This platform may be adaptable in terms of height, so as to adapt to differing body sizes. In addition, the second holding unit may have an inclination function, so as to enable optimal positioning of the breast.

The mammography unit may additionally comprise a special compression apparatus that may be attached to the second holding unit. This compression apparatus may be configured to compress the breast of the object under examination during the examination. Compression may improve image quality by reducing the thickness of the irradiated breast tissue of the object under examination and minimizing movement artifacts.

The thoracic wall, which plays an important role in mammography, may relate to a region of the object under examination where the breast adjoins the thoracic cage. The mammography unit may advantageously be so configured as to enable the breast of the object under examination to be mapped as completely as possible up to the thoracic wall. To this end, the X-ray unit, in particular the anode axis, may advantageously be at an angle of less than 90°, in particular 84°, to a vertical plane, in particular a vertical, of the stand unit, this being denoted “single-tank angle”. In particular, the anode axis may be at an angle of between 0° and 30°, in particular 6°, relative to the aperture plane of the aperture unit. This angle may help in achieving a more uniform X-ray intensity on a side close to the thoracic wall and in reducing the distance from the thoracic wall to the focal spot.

The control unit of the mammography unit may comprise specific algorithms for image processing and reconstruction. These algorithms may be configured to improve the visibility of microcalcifications and other subtle indications of breast cancer. The control unit may, moreover, implement functions for automatic exposure control (AEC), in order to ensure an optimal radiation dose for each individual breast.

Overall, through the combination of precise control of the electron beam by way of the coil, optimized geometry, and advanced image processing techniques, the described mammography unit is able to ensure high quality breast imaging with a reduced radiation dose.

In a further advantageous embodiment of the proposed imaging device, the first holding unit may be L- or J-shaped and arranged on the stand unit by way of a first leg. The X-ray unit may be arranged on a further leg of the first holding unit.

The first holding unit may be L- or J-shaped. An L-shaped configuration may comprise two legs arranged at right angles to one another. A J-shaped configuration may comprise a straight side and a curved side. The L- or J-shaped configuration may enable a compact construction of the imaging device. The first leg may take the form of the part of the L- or J-shape arranged in a vertical plane, in particular layer, and serve in attachment to the stand unit. The further leg may take the form of the part of the L- or J-shape arranged in a horizontal plane, in particular layer, in particular the form of the curved part of the J-shape.

The first leg may take the form of a part of the first holding unit which serves in connection with the stand unit. The first leg may take the form, for example, of an arm in a vertical plane, in particular layer. The arrangement of the first holding unit on the stand unit by way of the first leg may comprise a detachable or fixed connection between the first holding unit and the stand unit. The first leg may be arranged on the stand unit by way of various attachment mechanisms. For example, the first leg may be attached to the stand unit by way of screwed joints, bolted joints, welded joints, and/or clamped joints. The attachment may advantageously be embodied in a movable manner, in order to allow rotation, in particular swiveling movement, of the first holding unit.

The further leg may be take the form of an additional part of the first holding unit, serving to accommodate the X-ray unit. The further leg may, for example, take the form of an arm in a horizontal plane, in particular layer.

The arrangement of the X-ray unit on the further leg of the first holding unit may comprise a detachable or fixed connection between the X-ray unit and the further leg. The X-ray unit may be arranged by way of various attachment mechanisms, for example a screwed joint, a bolted joint, a welded joint, and/or a clamped joint. Positioning of the X-ray unit on the further leg may be selected such that the X-ray unit is in a suitable position and orientation relative to the X-ray detector.

The L- or J-shaped configuration of the first holding unit together with the arrangement of the X-ray unit on the further leg may advantageously enable flexible positioning of the X-ray unit relative to the X-ray detector. This may simplify adaptation to differing examination situations and patient sizes. The first leg may provide a stable, mobile connection to the stand unit, while the further leg may position the X-ray unit at a defined distance from the stand unit. This may enable flexible positioning of the X-ray unit without impairing the stability of the overall system. Arranging the X-ray unit on the further leg may result in greater freedom of movement for the X-ray unit. This may be advantageous in particular when carrying out tomosynthesis captures or other capture techniques in which the X-ray unit needs to move along a capture path. The L- or J-shaped configuration of the first holding unit may additionally enable a compact construction of the imaging device. The space required by the apparatus may thereby be reduced, which may be advantageous in particular in confined spaces. A further advantage of this embodiment may consist in the fact that the L- or J-shaped structure may enable improved accessibility to an examination region of the imaging device. This may simplify positioning of the object under examination and offer medical personnel greater freedom of movement when carrying out examinations.

In a further advantageous embodiment of the proposed imaging device, the X-ray unit may be movably bearing-mounted parallel to a plane of the stand unit. The plane of the stand unit may in particular be a vertical plane. The anode axis may be at a predefined angle relative to the plane of the stand unit. The predefined angle may amount to less than 90°, in particular 84°.

A vertical plane may be defined as a plane that is oriented perpendicular to the surface of the earth. The vertical plane may, for example, be defined by the direction of gravity and a horizontal direction. In this case, the vertical plane of the stand unit may advantageously adjoin the stand unit, touch the stand unit, or intersect with the stand unit.

The mobility of the X-ray unit parallel to the plane of the stand unit may enable movement in a direction parallel to the plane. The X-ray unit may be movably bearing-mounted parallel to the vertical plane of the stand unit. Movement of the X-ray unit may comprise translational and/or rotational movement of the X-ray unit. In this case, while the X-ray unit is in movement, a spatial distance between the X-ray unit and the vertical plane may advantageously remain constant, as may an angle between the anode axis and the vertical plane.

Making the predefined angle smaller than 90° may offer various technical advantages. Arranging the anode axis at an angle of less than 90° relative to the vertical plane of the stand unit allows an advantageous orientation of the X-ray beam emitted by the X-ray unit to be achieved. The inclination of the anode axis relative to the vertical plane of the stand unit may result in an improved beam geometry of the X-rays, which may result in a more uniform distribution of the X-rays on the X-ray detector. This may be advantageous in particular in the case of an examination of breast tissue, since the more uniform distribution of the X-rays on the X-ray detector may enable an improved representation of structures. In this respect, the specified angle may advantageously be specified such that, relative to the plane of the stand unit, the anode axis is arranged rising on one side of the X-ray unit and falling on a side remote from the X-ray unit.

A spatial distance between the focal spot and the thoracic wall may be in a direct relationship with the predefined angle between the anode axis and the vertical plane of the stand unit. The smaller the predefined angle between the anode axis and the vertical plane of the stand unit, the closer the focal spot may be brought to the thoracic wall. A reduction in the spatial distance between the focal spot and the thoracic wall may lead to improved mapping of the tissue close to the thoracic wall, which may be significant for diagnostic accuracy.

The specific embodiment with a predefined angle of 84° between the anode axis and the vertical plane of the stand unit may result in a particularly advantageous configuration. This angle may represent a good compromise between the approximation of the focal spot on the thoracic wall and the maintenance of an improved beam geometry. The 84° angle may result in a more uniform distribution of X-ray intensity on the side of an object under examination close to the thoracic wall. In addition, the predefined angle of 84° between the anode axis and the vertical plane of the stand unit may shorten the distance from the thoracic wall to the focal spot by around 10%.

The proposed embodiment may advantageously enable precise positioning and orientation of the X-ray unit. In addition, improved beam geometry and distribution of X-ray intensity may be achieved, which may result in improved image quality simultaneously alongside reduced radiation exposure for the object under examination.

A fifth aspect of one or more example embodiments of the present invention relates to a method for acquiring image data by way of a proposed imaging device. In a first step, the X-ray unit is moved relative to the X-ray detector along a capture path. The capture path specifies one or more capture segments for arranging the X-ray unit. In a further step, when arranged in the capture segment or in one of the multiple capture segments, the X-ray unit is operated according to a proposed method for operating an X-ray unit. In this respect, the X-ray detector acquires the X-rays emitted by way of the X-ray unit. In a further step, the energization of the coil during movement of the X-ray unit along the capture segment or in each case one of the multiple capture segments of the capture path is adapted as a function of an instantaneous position of the X-ray unit in such a way that the electron beam emitted by the cathode is deflected by way of the magnetic field to minimize any movement of the focal spot relative to the X-ray detector. In a further step, the image data is provided on the basis of the acquired X-rays.

The advantages of the proposed method for acquiring image data by way of an imaging device substantially correspond to the advantages of the proposed X-ray unit, the proposed imaging device, the proposed method for operating an X-ray unit, and/or the proposed method for calibrating an X-ray unit. Features, advantages, or alternative embodiments mentioned in this connection are likewise also applicable to the other claimed subjects and vice versa.

In particular, a proposed imaging device may be configured to carry out an embodiment of the proposed method for acquiring image data.

The capture path may, for example, comprise a specified trajectory along which the X-ray unit may be moved. The capture path may comprise the one capture segment or the multiple capture segments. The one or more capture segments may in each case comprise a position along the capture path or an, in particular spatial, segment of the capture path. In this case, to emit X-rays, the X-ray unit may be arranged within the capture segment or within in each case one of the multiple capture segments. The arrangement of the X-ray unit in the one or more capture segments may, for example, be controlled by a control unit of the imaging device. If the capture path comprises multiple capture segments, the capture segments may partly intersect or differ from one another. In particular, in each case two of the capture segments of the capture path may adjoin one another, in particular directly, or be arranged spaced from one another. In this case, the capture path may be at least spatially, in particular spatially and temporally, resolved. The capture path may, for example, comprise a time sequence of the multiple capture segments. By way of the one or multiple capture segments, the capture path may specify the trajectory along which the X-ray unit may be moved relative to the X-ray detector. The capture path may, for example, comprise a linear and/or arcuate and/or circular trajectory.

The X-ray unit may be moved relative to the X-ray detector along a capture path. When arranged in the capture segment or in one of the multiple capture segments, the X-ray unit is operated according to a method for operating an X-ray unit, in particular during movement of the X-ray unit along the capture segment or the respective one of the multiple capture segments.

The X-ray detector may acquire the X-rays emitted by way of the X-ray unit. The energization of the coil during movement of the X-ray unit along the capture segment or in each case along one of the multiple capture segments of the capture path may be adapted as a function of an instantaneous position of the X-ray unit in such a way that the electron beam emitted by the cathode is deflected by way of the magnetic field to minimize any movement of the focal spot relative to the X-ray detector. The image data may be provided on the basis of the acquired X-rays.

The method for operating the X-ray unit in the one or more capture segments may, for example, comprise providing a high voltage between the cathode and the anode plate, energizing the cathode to provide the electron beam, rotating the anode plate at a rotational frequency within a specified rotational frequency range, energizing the coil with a deflection current to provide the magnetic field, and adapting the deflection of the electron beam by adapting the energization of the coil.

Movement of the X-ray unit along the capture path may comprise relative movement between the X-ray unit and the X-ray detector. In this case, the X-ray unit may be arranged in a time sequence, in particular sequentially, in the multiple capture segments of the capture path.

The instantaneous position of the X-ray unit may describe a spatial position and/or orientation and/or posture of the X-ray unit, in particular relative to the X-ray detector. The instantaneous position of the X-ray unit may, for example, be acquired by way of a positioning sensor. Alternatively or additionally, the X-ray unit may be movably bearing-mounted by way of a calibrated movement unit. In this case, positioning information including information about the instantaneous position of the X-ray unit may be provided by the calibrated movement unit.

The energization of the coil may be adapted as a function of, in particular on the basis of, the instantaneous position of the X-ray unit. Adaptation of the energization of the coil as a function of the instantaneous position of the X-ray unit may involve determining and specifying, in particular adjusting, the deflection current for energization of the coil. Adaptation of the energization of the coil, in particular of the deflection current, may be based on stored, in particular saved, calibration information that characterizes a correlation between the deflection of the electron beam and the deflection current. The calibration information may have been provided, for example by way of a proposed method for calibrating an X-ray unit. By adapting the energization of the coil, in particular of the deflection current, a strength and/or direction of the magnetic field generated by the coil may be influenced.

Minimizing the movement of the focal spot relative to the X-ray detector may involve reducing or avoiding any relative repositioning between the focal spot and the X-ray detector. This may be advantageous in order to achieve a constant image quality during capture of image data, in particular X-ray image data, in particular in the case of capture sequences with a moving X-ray unit such as for example in the case of tomosynthesis.

The energization of the coil, in particular the deflection current, may advantageously be adapted as a function of relative movement between the X-ray unit and the X-ray detector, in order to achieve minimization of the movement of the focal spot. A change in the deflection current may result in a modification of the magnetic field generated by the coil, which may in turn influence the position of the electron beam and thus of the focal spot on the anode plate.

In the event of an increase in the deflection current, the magnetic field may grow in strength, so possibly leading to stronger deflection of the electron beam. This may bring about repositioning in the focal spot on the anode plate. Conversely, a reduction in the deflection current may lead to weaker deflection and repositioning of the focal spot in the opposing direction.

To compensate for a relative movement between the X-ray unit and the X-ray detector, the deflection current may be adapted, in particular continuously adapted. If, for example, the X-ray unit is moving in a given direction, the deflection current may be modified in such a way that the focal spot is shifted in an opposing direction. The deflection current may here be adapted proportionally to a speed and direction of the relative movement.

The instantaneous position of the X-ray unit may advantageously be acquired and the deflection current regulated accordingly, in particular in real time, in order to ensure a maximally constant position of the focal spot relative to the X-ray detector.

The energization of the coil may be adapted during movement of the X-ray unit along the capture path, in particular within the capture segment or in each case one of the capture segments and/or between in each case two successive, in particular adjacent, capture segments of the capture path, as a function of the instantaneous position of the X-ray unit. Energization may, for example, be adapted by way of the control unit of the imaging device.

Acquisition of the X-rays emitted by the X-ray unit may involve detecting the X-rays by way of the X-ray detector. Acquisition of the X-rays may, for example, involve converting the X-rays into electrical signals. The X-rays may, for example, be acquired by way of an X-ray-sensitive layer, in particular surface, of the X-ray detector. In particular, the X-rays may be acquired during the arrangement of the X-ray unit in the capture segment or in each case one of the multiple capture segments.

The image data may be provided by generating, in particular reconstructing, the image data on the basis of the acquired X-rays. Provision of the image data set may, for example, include saving the image data on a computer-readable storage medium and/or displaying the image data on a display unit and/or transferring it to a provision unit.

The proposed method may have the advantage that, by adapting the energization of the coil during movement of the X-ray unit, it is possible to achieve minimization of the movement of the focal spot relative to the X-ray detector. It is possible to achieve improved image quality of the acquired image data as a result.

In a further advantageous embodiment of the proposed method for acquiring image data by way of an imaging device, the coil may additionally be energized as a function of a speed of movement of the X-ray unit.

The speed of movement of the X-ray unit may describe a speed at which the X-ray unit is moving along the capture path. The speed of movement of the X-ray unit may, for example, be acquired by way of a sensor. The sensor may take the form of a speed sensor, acceleration sensor, and/or position sensor.

Energization of the coil as a function of the speed of movement may involve adapting the deflection current. The deflection current may be adapted in such a way that deflection of the electron beam counteracts movement of the X-ray unit, movement of the focal spot relative to the X-ray detector thus being minimized.

When it comes to energization of the coil as a function of the speed of movement, account may advantageously be taken of a time component. For example, a future position of the X-ray unit for a future point in time may be identified on the basis of an instantaneous position and an instantaneous speed of movement of the X-ray unit, in order proactively to adapt energization of the coil.

A rate of change of the energization of the coil, in particular of the deflection current, necessary for minimizing the movement of the focal spot relative to the X-ray detector may be directly related to the speed of movement of the X-ray unit. In the case of a relatively high speed of movement of the X-ray unit, a faster rate of change may be necessary for the deflection current if movement of the focal spot relative to the X-ray detector is to be effectively compensated.

The maximum rate of change of the deflection current may be limited by electrical characteristics of the coil, in particular by the inductance thereof. The inductance of the coil may define the speed at which the deflection current may be modified.

In the case of very high speeds of movement of the X-ray unit, it may be necessary to anticipate modification of the deflection current. To this end, it is possible to implement a predictive control algorithm that is configured to predict the future position of the X-ray unit on the basis of its instantaneous position and instantaneous speed and to undertake corresponding forward-looking adaptation of the deflection current.

Taking the speed of movement into account when energizing the coil may result in improved image quality, since movement of the focal spot relative to the X-ray detector may be more precisely minimized. This may be advantageous in particular in the event of rapid movement of the X-ray unit.

In a further advantageous embodiment of the proposed method for acquiring image data by way of an imaging device, the coil may be energized over a predefined period before the high voltage is provided between the cathode and the anode plate and/or the cathode is energized to provide the electron beam.

The predefined period may take the form of a time interval which is defined in advance. The predefined period may be defined as a function of the specific characteristics of the X-ray unit and the requirements of the imaging method. The predefined period may in particular be adapted as a function of a speed of movement of the X-ray unit. In the case of relatively high speeds of movement, a longer predefined period may be selected in order to ensure a sufficient lead time for build-up of the magnetic field. The predefined period may advantageously be adapted on the basis of a response time of the coil.

According to a first embodiment, energization of the coil may be provided over a predefined period before the high voltage is provided between the cathode and the anode plate. In this case, the coil may be supplied with the deflection current for the predefined period before the high voltage is applied between the cathode and the anode plate. This may advantageously make it possible to build up a stable magnetic field before the high voltage is activated. The predefined period may, for example, amount to a few milliseconds up to a number of seconds, depending on the specific system requirements. This time offset makes it possible to ensure that the magnetic field for deflecting the electron beam has already been fully built up when the high voltage is applied.

According to a further embodiment, the coil may be energized over a predefined period before the cathode is energized to provide the electron beam. In this case, the coil may be supplied with the deflection current for the predefined period before the cathode is activated to emit electrons. This approach may be advantageous in terms of ensuring that the magnetic field is already present when the electron beam is generated.

In a further embodiment, the coil may be energized over a predefined period both before the high voltage is provided between the cathode and the anode plate and before the cathode is energized to provide the electron beam. This combination may enable comprehensive preparation of the magnetic field. The magnetic field may be fully built up before both the high voltage is applied and the cathode is activated. This may result in particularly precise control of the electron beam from the moment it is generated. In this embodiment, the predefined period may be determined such that it takes account of the longest response time of the components involved.

Energization of the coil over the predefined period before the high voltage is provided and/or the cathode is energized may be advantageous in terms of building up a stable magnetic field before the electron beam is generated. This may enable more precise deflection of the electron beam. This may result in higher image quality when acquiring image data by way of the imaging device.

In a further advantageous embodiment of the proposed method for acquiring image data by way of an imaging device, an initial deflection current for energizing the coil prior to the start of movement of the X-ray unit may be specified as a function of the high voltage provided between the cathode and the anode plate and of a planned speed of movement of the X-ray unit.

An initial deflection current may be specified for energizing the coil prior to the start of movement of the X-ray unit. The initial deflection current may serve as a starting value for energization of the coil, in particular prior to the start of movement of the X-ray unit. This initial deflection current may be determined on the basis of various parameters, in particular the high voltage provided between the cathode and the anode plate and a planned speed of movement of the X-ray unit.

The deflection current may influence the strength and/or direction of the magnetic field generated by the coil. The magnetic field may furthermore deflect the electron beam. Deflection of the electron beam may influence characteristics of the focal spot, for example the orientation and/or shape of the focal spot. In the event of deflection of the electron beam, the shape, for example an outline and/or distribution, in particular an intensity distribution, of the focal spot on the anode plate may, for example be modified. In particular, magnification and/or distortion of the focal spot on the anode plate may occur if the electron beam impinges on the anode plate at a relatively steep angle.

The angle of the single-angle plate of the X-ray unit may likewise have an effect on the shape and size of the focal spot. A single-angle plate with an angle of 7° to 20°, in particular 16°, may help to improve the effective size of the focal spot.

Specifying an initial deflection current prior to the start of movement of the X-ray unit may bring about initial deflection of the focal spot, which may result in an optimized starting position of the focal spot on the anode plate. This may help the focal spot to exhibit a favorable, in particular compact and/or uniform, shape and/or position at the start of movement of the X-ray unit.

The initial deflection of the focal spot may, for example, help the focal spot to exhibit a compact and/or uniform shape during movement of the X-ray unit. This may in particular be advantageous in the event of rapid movements or complex capture paths, since it may enable stabler focal spot geometry throughout the entire capture sequence.

The shape of the focal spot may have a significant effect on image quality. A well-defined and/or compact focal spot may lead to higher spatial resolution of the image data, in particular X-ray image data. A distorted and/or enlarged focal spot may, in contrast, lead to a reduction in image sharpness.

An adapted, in particular optimized, focal spot shape may, furthermore, help in making the distribution of X-ray intensity more uniform. This may lead to improved contrast resolution and reduced artifact formation in the image data. In particular in the case of tomosynthesis captures, in which multiple projections from various angles are acquired, a consistent focal spot shape over the entire capture path may contribute to improved reconstruction quality.

Controlling and optimizing the focal spot shape by way of targeted deflection of the electron beam may thus result in an improvement in image quality. This may result in to more precise diagnostics and potentially reduced radiation exposure for the patient, since fewer repeat captures may possibly be required.

The initial deflection current may be specified as a function of the high voltage provided between the cathode and the anode plate. The high voltage provided may influence the energy of the electrons in the electron beam and thus influence the necessary deflection by the magnetic field of the coil. A higher high voltage may lead to less deflection of the electron beam with the same magnetic field. The initial deflection current may therefore be adapted accordingly in order to achieve a desired deflection of the electron beam.

Furthermore, the initial deflection current may be specified as a function of the planned speed of movement of the X-ray unit. The planned speed of movement may influence the necessary deflection of the electron beam for minimizing the movement of the focal spot relative to the X-ray detector. The planned speed of movement of the X-ray unit may influence the rate of change of the deflection current needed for minimizing the movement of the focal spot relative to the X-ray detector. In the event of a relatively high planned speed of movement, a higher rate of change of the deflection current may be necessary in order to minimize movement of the focal spot relative to the X-ray detector. The initial deflection current may advantageously be specified in such a way that it produces an optimal starting point for subsequent dynamic adaptation, in particular taking account of the necessary rate of change of the deflection current.

The initial deflection current may, for example, be specified with the assistance of calibration information. The calibration information may characterize a correlation between the deflection of the electron beam and the deflection current and/or between the deflection of the electron beam and the high voltage. The calibration information may advantageously be provided by way of a proposed method for calibrating an X-ray unit.

The initial deflection current may serve as a starting value for subsequent dynamic adaptation of the energization of the coil during movement of the X-ray unit. The dynamic adaptation may involve continuous or incremental modification of the deflection current.

Provision of the initial deflection current may involve energizing the coil with the initial deflection current.

In a further advantageous embodiment of the proposed method for acquiring image data by way of an imaging device, the X-ray unit may comprise a aperture unit. The aperture unit may be configured to spatially limit the X-rays exiting from the beam exit window. The aperture unit may be adapted within the one or more capture segments and/or between in each case successive capture segments of the capture path of the X-ray unit.

Adaptation of the aperture unit may involve adjusting an opening of the aperture unit. This may involve modifying the size and/or shape of the opening of the aperture unit. In this respect, the aperture unit may be adjusted for example in a motor-driven manner and/or electromechanically.

Adaptation of the aperture unit within the one or more capture segments may involve adjusting the aperture unit during movement of the X-ray unit within the respective capture segment, in particular during the emission of X-rays. In this case, the aperture unit may be adapted on the basis of the instantaneous capture segment. In particular, the aperture unit may be adapted on the basis of the relative positioning between the X-ray unit, in particular the focal spot, and the X-ray detector. The aperture unit may be adapted such that the aperture unit limits, in particular dynamically, the exiting X-rays, in particular during arrangement and/or movement of the X-ray unit along the respective capture segment, to an X-ray-sensitive layer, in particular surface, of the X-ray detector and/or a region of interest to be mapped of the object under examination. Adaptation of the aperture unit within the one or more capture segments may involve continuously and/or discretely adapting the aperture unit.

Adaptation of the aperture unit between in each case successive capture segments may involve adjusting the aperture unit between two temporally successive capture segments of the capture path, in particular while the X-ray unit is being moved between two successive capture segments. The aperture unit may be adapted on the basis of the capture path, in particular the in each case successive capture segment. In particular, the aperture unit may be adapted on the basis of the relative positioning between the X-ray unit, in particular the focal spot, and the X-ray detector. The aperture unit may be adapted such that the aperture unit limits the exiting X-rays to an X-ray-sensitive layer, in particular surface, of the X-ray detector and/or a region of interest to be mapped of the object under examination. Adaptation of the aperture unit between in each case successive capture segments may involve abrupt adaptation of the aperture unit.

Furthermore, the aperture unit may be adapted within the one or more capture segments and/or between in each case successive capture segments of the capture path of the X-ray unit in such a way that the X-rays partially cross-illuminate the X-ray detector as a function of the deflection of the focal spot.

Adaptation of the aperture unit may, furthermore, take account of the modification of the position of the focal spot by deflection of the electron beam. When the focal spot is repositioned on the anode plate, the aperture unit may be corrected appropriately in order to ensure optimal utilization of the detector area.

Furthermore, the aperture unit may be asymmetrically adjusted to adapt a beam geometry of the X-rays to specific capture situations. This may be of relevance, for example, in the case of tomosynthesis captures in which an angle between the X-ray unit and the X-ray detector changes along the capture path.

By adapting the aperture unit within the one or more capture segments and/or between in each case successive capture segments of the capture path of the X-ray unit, it is advantageously possible to achieve improved spatial limitation of the X-rays exiting through the beam exit window. In this way, radiation exposure may be further reduced for the object under examination. Furthermore, improved image quality of the acquired image data may hereby by achieved.

Precise adaptation of the aperture unit may help to reduce stray radiation and improve image quality. At the same time, optimized superimposition of the X-rays may minimize radiation exposure for the object under examination.

According to one variant, the aperture unit may advantageously not be adapted, in particular corrected, during X-ray acquisition, in particular during X-ray detector readout, but instead the aperture unit may be adapted between the individual captures, in particular the multiple capture segments.

In a further advantageous embodiment of the proposed method for acquiring image data by way of an imaging device, the aperture unit may be adapted within the one or more capture segments of the capture path contrary to a direction of movement of the X-ray unit.

The direction of movement may be a spatial direction in which the X-ray unit moves within the respective capture segment of the capture path. Adaptation of the aperture unit contrary to the direction of movement of the X-ray unit may involve adapting the aperture unit in a direction opposite to the direction of movement of the X-ray unit. Adaptation of the aperture unit contrary to the direction of movement may have the effect of making spatial limitation of the X-rays counteract movement of the X-ray unit. In particular, adaptation of the aperture unit contrary to the direction of movement of the X-ray unit may cause the X-rays to be more strongly limited on one side along the direction of movement of the X-ray unit and less limited on an opposing side. This may, for example, be performed by way of the aperture unit through reduction of the radiation field in the direction of movement and/or expansion of the radiation field contrary to the direction of movement.

The aperture unit may be adapted continuously or incrementally within the one or more capture segments. In the case of continuous adaptation, the aperture unit may be adapted uninterruptedly during movement of the X-ray unit. In the case of incremental adaptation, the aperture unit may be adapted in discrete steps within the one or more capture segments.

Adapting the aperture unit contrary to the direction of movement of the X-ray unit may result in improved spatial limitation of the exiting X-rays. This may help to reduce radiation exposure for an object under examination and simultaneously improve image quality.

In a further advantageous embodiment of the proposed method for acquiring image data by way of an imaging device, the aperture unit may be adapted between in each case successive capture segments of the capture path along a direction of movement of the X-ray unit.

The direction of movement may be a spatial direction in which the X-ray unit moves within the respective capture segment of the capture path. Adaptation of the aperture unit along the direction of movement may involve adapting the aperture unit in the direction of movement of the X-ray unit. This may advantageously enable the aperture unit to already be correctly positioned for the in each case next, in particular temporally subsequent, capture segment. Successive capture segments may be segments of the capture path which succeed one directly another temporally or spatially. The X-ray unit may be moved between the successive capture segments without image data being captured.

In particular, adaptation of the aperture unit along the direction of movement of the X-ray unit may cause the X-rays to be less limited on one side along the direction of movement of the X-ray unit and more strongly limited on an opposing side. This may, for example, be performed by way of the aperture unit through reduction of the radiation field contrary to the direction of movement and/or expansion of the radiation field along the direction of movement.

Adaptation of the aperture unit between the capture segments may advantageously make it possible for optimal adjustment of the aperture unit to be performed for each capture segment. This may lead to improved image quality and/or reduced radiation exposure for the object under examination.

In a further advantageous embodiment of the proposed method for acquiring image data by way of an imaging device, the cathode may be energized in a pulsed manner to provide the electron beam each time the X-ray unit is arranged in one of the one or more capture segments.

Energization of the cathode to provide the electron beam may involve applying an electrical current to the cathode. Energization may here be performed in a pulsed manner. In this case, the current may be applied to the cathode in short, successive current pulses. Pulsed energization may involve a sequence of current pulses, wherein each current pulse may have a defined pulse duration and pulse amplitude. Intervals in which no or only a small current flows may be inserted between the current pulses. Pulsed energization of the cathode may be adaptable through specification of a pulse duration, pulse frequency, and/or pulse amplitude. The pulse frequency, pulse duration, and pulse amplitude of the pulsed energization of the cathode may advantageously be adapted to the respective capture requirements. The pulse duration may, for example, be in the range of micro- or milliseconds. The pulse frequency may be adapted to the speed of movement of the X-ray unit and the desired spatial resolution. A higher pulse frequency may enable finer spatial sampling, but may possibly require faster movement of the X-ray unit. The pulse amplitude of the current pulses may be adapted to a desired X-ray intensity. Greater pulse amplitudes, in particular current amplitudes, may lead to higher intensity X-rays, but also increase thermal loading of the cathode and the anode plate.

A longer pulse duration may increase the total dose and lead to improved image quality. A longer pulse duration may furthermore lead to heavier thermal loading of the anode plate. A longer pulse duration may furthermore increase the risk of motion blur in the case of moving objects. A shorter pulse duration may reduce the total dose and may decrease radiation exposure for the object under examination. Furthermore, a shorter pulse duration may reduce thermal loading of the anode plate. A shorter pulse duration may reduce motion artifacts but may increase noise in the image.

Pulsed energization of the cathode may advantageously be synchronized with movement of the X-ray unit along the capture path, in particular with the arrangement of the X-ray unit in the capture segment or in in each case one of the multiple capture segments. The current pulses may advantageously be provided to the cathode in such a way that they occur at precisely the moment that the X-ray unit is located in the capture segment or in one of the multiple capture segments. This may enable precise spatial correlation of the acquired X-rays.

Pulsed energization may, furthermore, be synchronized with the rotation of the anode plate. This may make it possible for the electron beam to impinge on a cooled region of the anode plate, so possibly reducing thermal loading and extending the service life of the anode plate.

Pulsed energization of the cathode may advantageously enable precise control of electron beam generation. Pulsed energization of the cathode on arrangement of the X-ray unit in the capture segment or in one of the multiple capture segments may result in precise temporal and spatial control of X-ray emission. This may lead to improved image quality and reduced radiation exposure for the object under examination. Furthermore, pulsed energization of the cathode may advantageously reduce thermal loading of the cathode, since cooling may occur in the intervals between current pulses. This may extend the service life of the cathode. Furthermore, pulsed energization of the cathode may enable the emitted X-ray dose to be precisely controlled. In addition, the efficiency of the imaging method may be increased, since the X-rays are purposefully emitted only in the relevant capture segments.

In a further advantageous embodiment of the proposed method for acquiring image data by way of an imaging device, provision of the image data may involve reconstructing the image data on the basis of the acquired X-rays and of information relating to the capture segment or the respective capture segments of the multiple capture segments of the X-ray unit relative to the X-ray detector.

Reconstruction of the image data may involve processing the acquired X-rays. Reconstruction may be performed, for example, using a reconstruction unit. The reconstruction unit may be configured as part of the control unit or as a separate unit.

The information about the capture segment or about the respective capture segments of the multiple capture segments of the X-ray unit relative to the X-ray detector may comprise information about the spatial positions and/or orientations and/or poses of the X-ray unit and/or of the X-ray detector, in particular relative to one another. This information may, for example, be acquired using sensors and/or be derived from the movement of the X-ray unit on the basis of control signals.

The acquired X-rays may be provided by the X-ray detector to the reconstruction unit as raw data. The raw data may in this case comprise information about the intensity of the detected X-rays at various positions of the X-ray detector, in particular of the X-ray-sensitive layer of the X-ray detector.

Reconstruction on the basis of the acquired X-rays, in particular the raw data, and the information about the capture segment or the respective capture segments of the multiple capture segments may involve applying a reconstruction algorithm, in particular a filtered backprojection, an iterative reconstruction method, and/or a tomographic reconstruction. In this case, the image data may be reconstructed as three-dimensional volume data. Alternatively or additionally, the image data may be reconstructed as two-dimensional projection images.

Precise spatial correlation of the acquired X-rays may advantageously be enabled by reconstruction on the basis of the information about the capture segment or the respective capture segments of the multiple capture segments. This may lead to improved image quality of the reconstructed image data.

The reconstructed image data may then be saved in a suitable image format and provided for further processing and/or display. The image data may, for example, be present in the form of three-dimensional data sets or tomographic images.

A technical advantage of this method may consist in the fact that, by taking account of the precise capture segments during reconstruction, it is possible to achieve elevated image quality and spatial resolution. In addition, reconstruction may enable efficient utilization of the captured data and thus help in reducing the radiation dose for the object under examination.

A sixth aspect of one or more example embodiments of the present invention relates to a computer program product having a computer program that can be loaded directly into a memory of a control unit. The computer program product may comprise program parts. The program parts may be configured to carry out all the steps of a method for operating an X-ray unit, a method for calibrating an X-ray unit, and/or a method for acquiring image data by way of an imaging device. The program parts may be executed on execution of the program parts by the control unit.

The computer program product may comprise software with source code that has yet to be compiled and linked or has merely to be interpreted, or executable software code that has merely to be loaded into the control unit for execution. By way of a control unit, the computer program product may rapidly, identically repeatably and robustly carry out the method for operating an X-ray unit, the method for calibrating an X-ray unit, and/or the method for acquiring image data by way of an imaging device. The computer program product can be configured such that it may carry out the method steps, according to one or more example embodiments of the present invention, by way of the control unit.

The computer program product is for example saved on a computer-readable storage medium or saved to a network or server, from where it may be loaded into the processor of a control unit which may be directly connected to the control unit or may be configured as part of the control unit. Control information for the computer program product may furthermore be saved on an electronically readable data storage medium. The control information of the electronically readable data storage medium may be designed such that, when the data storage medium is used in a control unit, it carries out a method according to one or more example embodiments of the present invention. Examples of electronically readable data storage media may be a DVD, a magnetic tape, a hard disk, or a USB stick on which electronically readable control information, in particular software, may be saved. If this control information is read from the data storage medium and saved in a control unit, all the embodiments according to the present invention of the above-described methods may be carried out.

A largely software-based implementation may have the advantage that control units that are already in service may straightforwardly be retrofitted to operate in the manner according to one or more example embodiments of the present invention by way of a software update. In addition to the computer program, such a computer program product may optionally comprise additional elements such as for example documentation and/or additional components, as well as hardware components, such as for example hardware keys (dongles etc.) for using the software.

Further advantageous embodiments of the present invention are described below.

Embodiment A1: X-ray unit as claimed in one of claims 1 to 12, wherein the anode plate takes the form of a single-angle plate.

Embodiment A2: X-ray unit according to embodiment A1, wherein the single-angle plate has an angle of 7 to 20°, in particular 16°.

Embodiment A3: X-ray unit as claimed in one of claims 1 to 12 or according to embodiment A1 or A2,
wherein the anode plate has a diameter of 50 mm to 200 mm, in particular 90 mm.

Embodiment A4: X-ray unit as claimed in one of claims 1 to 12 or according to one of embodiments A1 to A 3,
wherein the anode plate comprises a support substrate, wherein a layer of a further substrate is applied on a side facing the cathode.

Embodiment A5: X-ray unit according to embodiment A4, wherein the support substrate comprises titanium-zirconium-molybdenum.

Embodiment A6: X-ray unit according to embodiment A4 or A5,
wherein the further substrate comprises a tungsten-rhenium alloy.

Embodiment A7: X-ray unit as claimed in one of claims 1 to 12 or according to one of embodiments A1 to A6,
wherein the cathode and the anode plate are at an average distance of 5 mm to 50 mm, in particular 8 mm.

Embodiment A8: X-ray unit as claimed in one of claims 1 to 12 or according to one of embodiments A1 to A7,
wherein the vacuum housing has respective feedthroughs for energizing the cathode and the rotating anode.

Embodiment A9: X-ray unit as claimed in one of claims 1 to 12 or according to one of embodiments A1 to A8,
wherein the vacuum housing is form from a substrate that comprises a glass-ceramic.

Embodiment A10: X-ray unit as claimed in one of claims 1 to 12 or according to one of embodiments A1 to A9,
furthermore comprising a high-voltage unit that is configured to provide a high voltage to the X-ray tube,
wherein the high-voltage unit is additionally arranged within the single-tank housing.

Embodiment A11: X-ray unit according to embodiment A10, wherein the single-tank housing has respective feedthroughs for providing energization to the high-voltage unit and the coil.

Embodiment A12: X-ray unit as claimed in one of claims 1 to 12 or according to one of embodiments A1 to A11,
wherein the magnetic field is configured to deflect the electron beam such that the focal spot on the anode plate is displaceable by up to 2.5 mm relative to a zero position.

Embodiment A13: X-ray unit as claimed in one of claims 1 to 12 or according to one of embodiments A1 to A12,
wherein a turn count of the coil is adapted as a function of a specified maximum deflection of the electron beam.

Embodiment A14: X-ray unit as claimed in claim 10 or as claimed in claim 10 and according to one of embodiments A1 to A13,
wherein the wall portion of the vacuum housing comprises a chromium-nickel steel alloy.

Embodiment A15: X-ray unit according to embodiment A14, wherein the chromium-nickel steel alloy takes the form of an austenitic steel alloy.

Embodiment A16: X-ray unit as claimed in one of claims 1 to 12 or according to one of embodiments A1 to A15,
wherein the cathode has at least one emitter that is configured to emit the electron beam.

Embodiment A17: X-ray unit according to embodiment A16,
wherein the at least one emitter takes the form of a flat emitter that has a slotted emitter plate for emitting the electron beam.

Embodiment A18: X-ray unit as claimed in one of claims 1 to 12 or according to one of embodiments A1 to A17,
wherein the cathode has two differently configured emitters, each of which is configured to emit an electron beam.

Embodiment A19: X-ray unit as claimed in claim 11 or 12 or as claimed in claim 11 or 12 and according to one of embodiments A1 to A18,
wherein the focusing head is of one-piece construction.

Embodiment A20: X-ray unit as claimed in claim 11 or 12 or according to embodiment A19,
wherein the focusing head comprises a chromium-nickel steel alloy or a molybdenum alloy.

Embodiment A21: X-ray unit according to embodiment A18 and as claimed in one of claims 11 or 12 or according to embodiment A18 and one of embodiments A19 or A20,
wherein the focusing head is configured for focusing in each case one or both of the electron beams that can be emitted by the two emitters.

Embodiment A22: X-ray unit as claimed in one of claims 1 to 12 or according to one of embodiments A1 to A21,
wherein the vacuum housing has, on a cathode-side wall, an at least partially recessed pump capillary for generating a vacuum in an inner region of the vacuum housing.

Embodiment A23: X-ray unit as claimed in one of claims 1 to 12 or according to one of embodiments A1 to A22,
wherein the rotatable bearing mount of the anode plate comprises an oscillation compensation.

Embodiment A24: X-ray unit as claimed in one of claims 1 to 12 or according to one of embodiments A1 to A23,
wherein the X-ray tube furthermore comprises a drive unit for motor-driven rotation of the anode plate.

Embodiment B1: method as claimed in claim 13 or 14,
wherein the cathode has two differently configured emitters, each of which is configured to emit an electron beam, wherein the emitters are selectively or simultaneously energized.

Embodiment B2: method as claimed in one of claims 13 or 14 or according to embodiment B1,
wherein the specified rotational frequency range comprises rotational frequencies from 130 Hz to 160 Hz.

Embodiment B3: method as claimed in one of claims 13 or 14 or according to embodiment B1 or B2,
wherein it is monitored whether the instantaneous rotational frequency of the anode plate is within the specified rotational frequency range,
wherein a warning is provided in the negative case.

The proposed method for operating an X-ray unit, in particular as claimed in claim 13 or 14 or according to one of embodiments B1 to B3, may in particular also be configured for operating an X-ray unit according to one of embodiments A1 to A24.

Embodiment C1: method for calibrating an X-ray unit as claimed in one of claims 1 to 12 or according to one of embodiments A1 to A24 comprising:
a) positioning an absorption mask between the beam exit window of the X-ray unit and an X-ray acquisition unit,
b) providing a high voltage between the cathode and the anode plate,
c) energizing the cathode to provide the electron beam,
d) rotating the anode plate at a rotational frequency within a specified rotational frequency range,
e) energizing the coil with a deflection current to provide the magnetic field,
f) acquiring a position of a mapping of the absorption mask by way of the X-ray acquisition unit,
   wherein steps b) to f) are carried out repeatedly for a plurality of differing high voltages and/or deflection currents,
   wherein a correlation between the deflection of the electron beam and the deflection current and/or between the deflection of the electron beam and the high voltage is identified on the basis of the plurality of differing high voltages and/or deflection currents and the associated positions of the mapping,
g) providing calibration information corresponding to the correlation.

Embodiment C2: method according to embodiment C1,
wherein the X-ray acquisition unit comprises an X-ray camera and/or an X-ray detector.

Embodiment C3: method according to embodiment C1 or C2,
wherein at least four differing high voltages and/or deflection currents are in each case specified for repeatedly carrying out steps b) to f).

Embodiment C4: method according to one of embodiments C1 to C3,
wherein identifying the correlation between the deflection of the electron beam and the deflection current and/or between the deflection of the electron beam and the high voltage of the electron beam involves a respective interpolation.

Embodiment D1: imaging device as claimed in claim 15 or 16,
furthermore comprising a stand unit, a first and a second holding unit,
wherein the first holding unit is arranged in a rotatably bearing-mounted manner on the stand unit,
wherein the X-ray unit is arranged on the first holding unit,
wherein the second holding unit is arranged on the stand unit,
wherein the X-ray detector is arranged on the second holding unit,
wherein the first and the second holding units hold the X-ray unit and the X-ray detector opposite one another.

Embodiment D2: imaging device according to embodiment D1,
wherein the first holding unit is L- or J-shaped and arranged on the stand unit by way of a first leg,
wherein the X-ray unit is arranged on a further leg of the first holding unit.

The proposed imaging device, in particular as claimed in claim 15 or 16 or according to one of embodiments D1 or D2, may in particular also comprise an X-ray unit according to one of embodiments A1 to A24.

### Embodiment E1: method as claimed in one of claims 17 to 20,

wherein the coil is additionally energized as a function of a speed of movement of the X-ray unit.

Embodiment E2: method as claimed in one of claims 17 to 20 or according to embodiment E1,
wherein the coil is energized over a predefined period before the high voltage is provided between the cathode and the anode plate and/or the cathode is energized to provide the electron beam.

Embodiment E3: method as claimed in one of claims 17 to 20 or according to one of embodiments E1 or E2,
wherein an initial deflection current for energizing the coil prior to the start of movement of the X-ray unit is specified as a function of the high voltage provided between the cathode and the anode plate and of a planned speed of movement of the X-ray unit.

Embodiment E4: method as claimed in one of claims 17 to 20 or according to one of embodiments E1 or E3,
wherein the cathode is energized in a pulsed manner to provide the electron beam each time the X-ray unit is arranged in one of the one or more capture segments of the capture path.

Embodiment E5: method as claimed in one of claims 17 to 20 or according to one of embodiments E1 or E4,
wherein provision of the image data may involve reconstructing the image data on the basis of the acquired X-rays and of information relating to the capture segment or the respective capture segments of the multiple capture segments of the X-ray unit relative to the X-ray detector.

The proposed method for acquiring image data, in particular as claimed in one of claims 17 to 20 or according to one of embodiments E1 to E5, may in particular also be configured to acquire image data by way of an imaging device according to one of embodiments D1 or D2. In addition, in one advantageous embodiment of the proposed method for acquiring image data, in particular as claimed in one of claims 17 to 20 or according to one of embodiments E1 to E4, the X-ray unit is operated according to the method according to one of embodiments B1 to B3 when it is arranged in the capture segment or in one of the multiple capture segments.

Embodiment F: computer program product having a computer program that can be loaded directly into a memory of a control unit, having program parts for carrying out all the steps of a method according to one of embodiments B1 to B3, C1 to C4 and/or E1 to E5 on execution of the program parts by the control unit.

### BRIEF DESCRIPTION OF THE DRAWINGS

Exemplary embodiments of the present invention are shown in the drawings and described in greater detail below. Identical reference signs are used for identical features in different figures, in which:

FIGS. 1 to 3 show schematic representations of various advantageous embodiments of a proposed X-ray unit,

FIG. 4 shows a schematic representation of a cross-section of an advantageous embodiment of the coil perpendicular to a winding direction of the coil,

FIG. 5 shows a schematic representation of a cross-section of an advantageous embodiment of the coil parallel to a winding direction of the coil,

FIG. 6 shows a schematic representation of a further advantageous embodiment of a proposed X-ray unit,

FIG. 7 shows a schematic representation of an advantageous embodiment of a cathode,

FIGS. 8 to 10 show schematic representations of various advantageous embodiments of a proposed method for operating an X-ray unit,

FIG. 11 shows a schematic representation of an advantageous embodiment of a proposed method for calibrating an X-ray unit,

FIGS. 12 to 16 show schematic representations of advantageous embodiments of a proposed imaging device,

FIGS. 17 to 20 show schematic representations of various advantageous embodiments of a proposed method for acquiring image data by way of an imaging device,

FIG. 21 shows a schematic representation of an exemplary deflection current-time curve,

FIG. 22 shows a schematic representation of an exemplary sequence diagram for use in a proposed method for acquiring image data by way of an imaging device.

### DETAILED DESCRIPTION

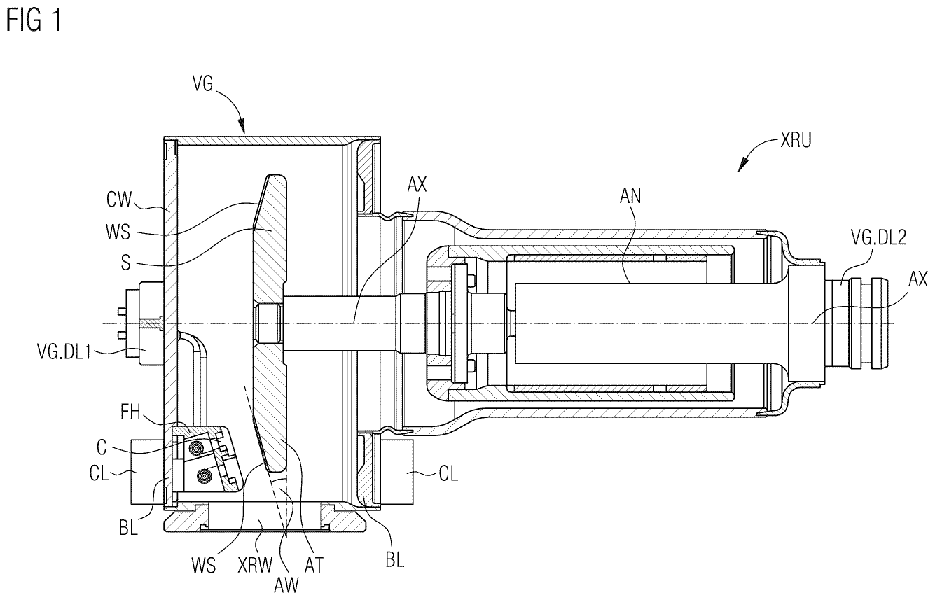

FIG. 1 shows a schematic representation of an advantageous embodiment of a proposed X-ray unit XRU. The X-ray unit may comprise an X-ray tube and a coil CL. The X-ray tube may here comprise a cathode C, a rotating anode, and a vacuum housing VG. The rotating anode may comprise an anode plate AT rotatably bearing-mounted about an anode axis AX. The cathode C and the rotating anode, in particular the anode plate AT, may additionally be arranged within the vacuum housing VG. The coil CL may be arranged outside the vacuum housing VG. The coil CL and the X-ray tube may furthermore be electrically isolated from one another. The coil CL may be configured to generate a magnetic field that may permeate an interspace between the cathode C and the anode plate AT. The magnetic field may be configured to deflect an electron beam originating from the cathode C and impinging on the anode plate AT in a focal spot of the X-ray tube. The vacuum housing VG may have a beam exit window XRW on a side arranged radially relative to the anode axis AX, which beam exit window is configured to allow passage of X-rays originating from the focal spot on the anode plate AT.

The anode plate AT may advantageously take the form of a single-angle plate. The angle plate may have an angle AW of 7° to 20°, in particular 16°. The anode plate AT may have a diameter of 50 mm to 200 mm, in particular 90 mm.

The anode plate AT may advantageously comprise a support substrate S. A layer of a further substrate WS may here be applied on a side facing the cathode C. The support substrate S may comprise titanium-zirconium-molybdenum. The further substrate WS may comprise a tungsten-rhenium alloy.

The cathode C and the anode plate AT may be at an average distance of 5 mm to 50 mm, in particular 8 mm, from one another.

The vacuum housing VG may have respective feedthroughs VG.DL1 and VG.DL2 for energizing the cathode C and the rotating anode. The feedthroughs VG.DL1 for energizing the cathode C may advantageously be arranged on a cathode-side wall CW of the vacuum housing VG.

The vacuum housing VG may be formed from a substrate that may comprise a glass-ceramic.

The magnetic field may be configured to deflect the electron beam such that the focal spot on the anode plate AT is displaceable by up to 2.5 mm relative to a zero position.

The electron beam may be arranged at least in part within an opening region of the coil CL when the X-ray unit XRU is in an operating state. In particular, the focal spot of the X-ray tube may be arranged within the opening region of the coil CL when the X-ray unit is in the operating state. The coil CL may take the form of an air-core coil. In addition, the turn count of the coil CL may be adapted as a function of a specified maximum deflection of the electron beam. The coil CL and the electron beam may be isolated from one another by a vacuum housing VG wall portion BL formed from a non-magnetic metallic material of low electrical conductivity. The wall portion BL of the vacuum housing VG may comprise a chromium-nickel steel alloy. The chromium-nickel steel alloy may take the form of an austenitic steel alloy.

The cathode C may have a focusing head FH for focusing the electron beam. The focusing head FH may in particular be of one-piece construction. The focusing head FH may furthermore comprise a chromium-nickel steel alloy or a molybdenum alloy.

The rotatable bearing mount of the anode plate AT may advantageously comprise an oscillation compensation.

The X-ray tube may furthermore comprise a drive unit AN for motor-driven rotation of the anode plate AT.

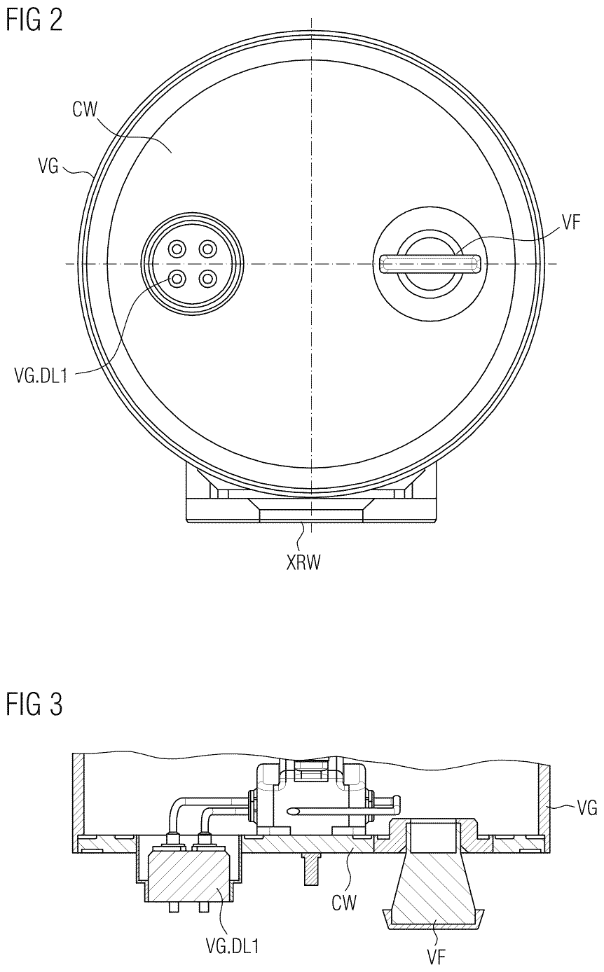

FIG. 2 shows a plan view onto the cathode-side wall CW of the vacuum housing VG of the embodiment of the proposed X-ray unit XRU shown schematically in FIG. 1. On the cathode side, the vacuum housing VG may have a substantially round, in particular circular, external shape, in particular outline. The anode axis AX may, for example, here run through a center point of the cathode-side wall CW of the vacuum housing.

In this case, the feedthroughs VG.DL1 for energizing the cathode C may be arranged in a spatial region, in particular in a symmetrical arrangement, of the cathode-side wall CW of the vacuum housing. In the embodiment shown in FIG. 2, the cathode C may have two emitters, in particular flat emitters. In this case, the X-ray unit XRU may in each case have two feedthroughs VG.DL1 for separately energizing the two emitters. As shown schematically in FIG. 2, the four feedthroughs VG.DL1 may, for example, be arranged within a circular recess in the cathode-side wall CW of the vacuum housing VG. The feedthroughs VG.DL1 may in this case, for example, be arranged at four corner points of a square.

The vacuum housing VG may furthermore have on the cathode-side wall CW an at least partially recessed pump capillary VF for generating a vacuum in an inner region of the vacuum housing VG.

The at least partially recessed pump capillary VF and the arrangement of the feedthroughs VG.DL1 for energizing the cathode C may, for example, be arranged on a common diameter of the cathode-side wall CW of the vacuum housing. The common diameter may here have a right-hand angle relative to the radially arranged beam exit window XRW of the vacuum housing VG.

FIG. 3 shows a view of a cross-section of the cathode-side wall CW of the vacuum housing VG of the embodiment of the proposed X-ray unit XRU shown schematically in FIGS. 1 and 2, in particular along the common diameter of the cathode-side wall CW along which is arranged the arrangement of feedthroughs VD.DL1 for energizing the cathode C and the pump capillary VF.

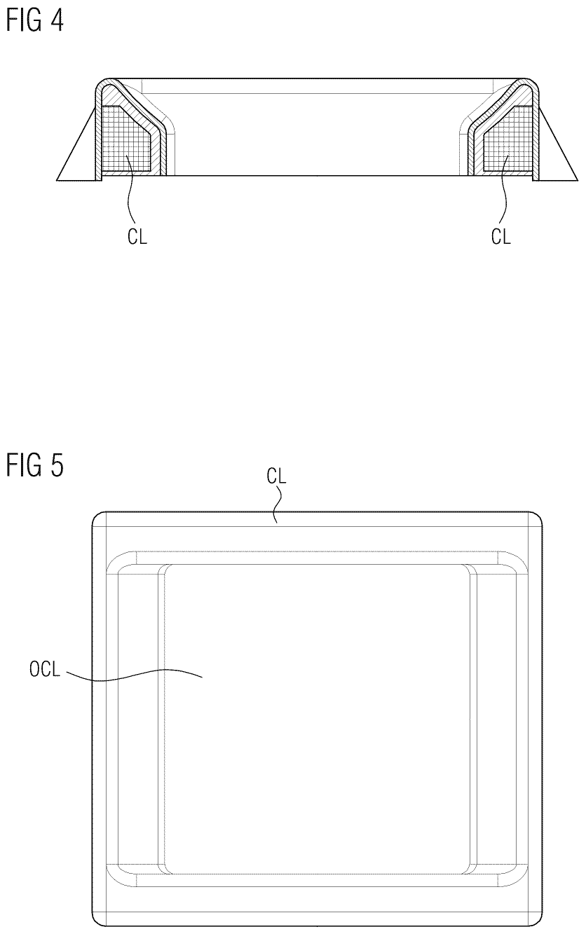

FIG. 4 shows a schematic representation of a cross-section of an advantageous embodiment of the coil CL perpendicular to a winding direction of the coil CL. The coil CL may here have a coil geometry that is adapted to a geometry of the X-ray tube, in particular of the vacuum housing VG. The coil geometry may advantageously comprise a cross-sectional geometry of the coil CL. The cross-sectional geometry of the coil CL may in this case be adapted at least on an inner side of the coil CL to the geometry of the vacuum housing VG within an opening region of the coil CL. The coil CL may furthermore take the form of an air-core coil. In the embodiment shown schematically in FIG. 4, a winding direction of the coil CL may run perpendicular to a plane of the drawing of the representation, in particular to a cross-sectional area of the cross-section. The cross-sectional geometry of the coil may here be trapezoidal at least in places.

FIG. 5 shows a schematic representation of a cross-section of an advantageous embodiment of the coil CL parallel to a winding direction of the coil CL. The coil CL may here have a coil housing that surrounds, in particular encloses, the coil CL. The coil housing may substantially follow the coil geometry, in particular an external shape, in particular outline, of the coil CL. In the embodiment shown schematically in FIG. 5, the coil CL may be arranged, in particular wound, substantially rectangularly, in particular squarely, around its opening region OCL. The coil CL, in particular the coil housing, may have a bevel, in particular a trapezoidal cross-section, on two opposing inner sides relative to the opening region OCL. In this way, the coil geometry, in particular the cross-sectional geometry, on the inner side of the coil CL may be adapted to the geometry of the vacuum housing VG within the opening region OCL of the coil CL.

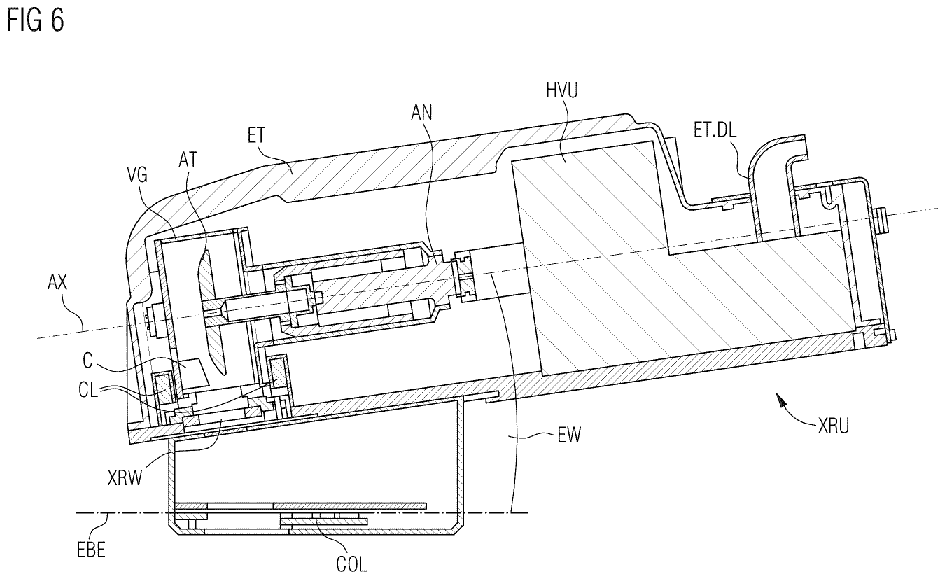

FIG. 6 shows a schematic representation of a further advantageous embodiment of a proposed X-ray unit XRU. The X-ray unit XRU may here furthermore comprise a leak-tight single-tank housing ET, wherein the X-ray tube and the coil CL may be arranged within the single-tank housing ET. The single-tank housing ET may furthermore be filled with a cooling/insulating medium.

The X-ray unit XRU may furthermore comprise a high-voltage unit HVU that may be configured to provide the high voltage to the X-ray tube, in particular the cathode C and the anode plate AT. In this case, the high-voltage unit HVU may additionally be arranged within the single-tank housing ET.

The single-tank housing ET may advantageously have respective feedthroughs ET.DL for providing energization of the high-voltage unit HVU and the coil CL.

The X-ray unit XRU may furthermore comprise a aperture unit COL that may be configured to spatially limit the X-rays exiting through the beam exit window XRW. The anode axis AX may in this case have a specified angle EW, in particular 6°, relative to an aperture plane EBE of the aperture unit COL. The inclination of the anode axis AX relative to the aperture plane EBE by the specified angle EW makes it possible to reduce the distance between the focal spot and the thoracic wall of an object under examination. For a specified angle EW of 6°, the distance between the focal spot and the thoracic wall may be shortened by around 10%.

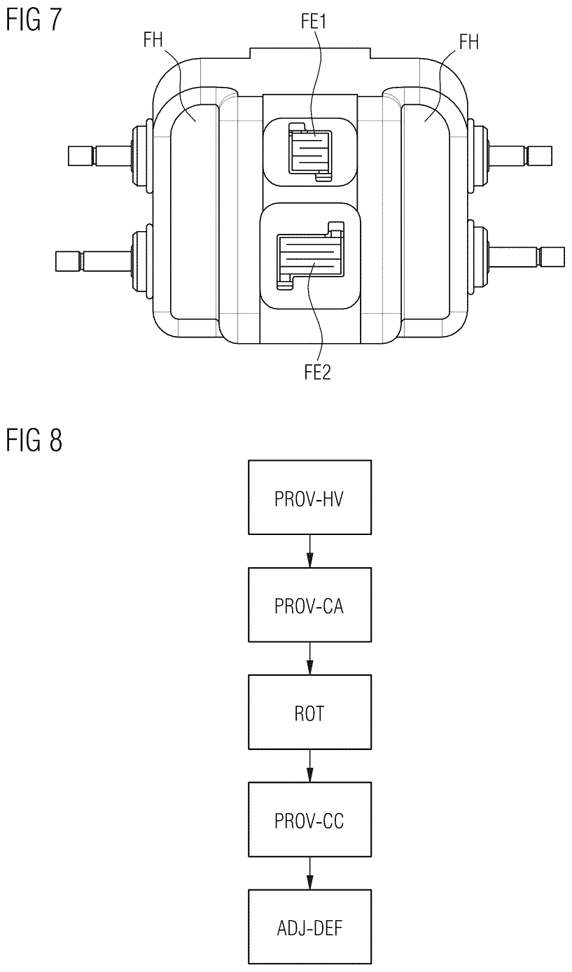

FIG. 7 shows a schematic representation of an advantageous embodiment of a cathode C. The cathode C may advantageously have two differently configured flat emitters FE1 and FE2, each made up of a slotted emitter plate. The two flat emitters FE1 and FE2 may in this case each be configured to emit an electron beam. The cathode C may have a focusing head FH for focusing the electron beam. In this case, the focusing head FH may be of one-piece construction. The focusing head FH may additionally be configured for focusing in each case one or both of the electron beams that can be emitted by the two flat emitters FE1 and FE2. In particular, the focusing head FH may be configured at least by its shape for electrostatically focusing the electron beam.

FIG. 8 shows a schematic representation of an advantageous embodiment of a proposed method for operating a proposed X-ray unit XRU. A high voltage may here be provided PROV-HV between the cathode C and the anode plate AT. The cathode C may be energized PROV-CA to generate an electron beam. The anode plate AT may furthermore be rotated at a rotational frequency within a specified rotational frequency range. The coil CL may furthermore be energized PROV-CC with a deflection current in order to generate a magnetic field. Deflection of the electron beam may additionally be adapted by adapting ADJ-DEF the energization of the coil CL.

The cathode C may advantageously have two differing emitters, in particular flat emitters FE1 and FE2, each of which is configured to emit an electron beam. The two emitters may in this case be selectively or simultaneously energized PROV-CA.

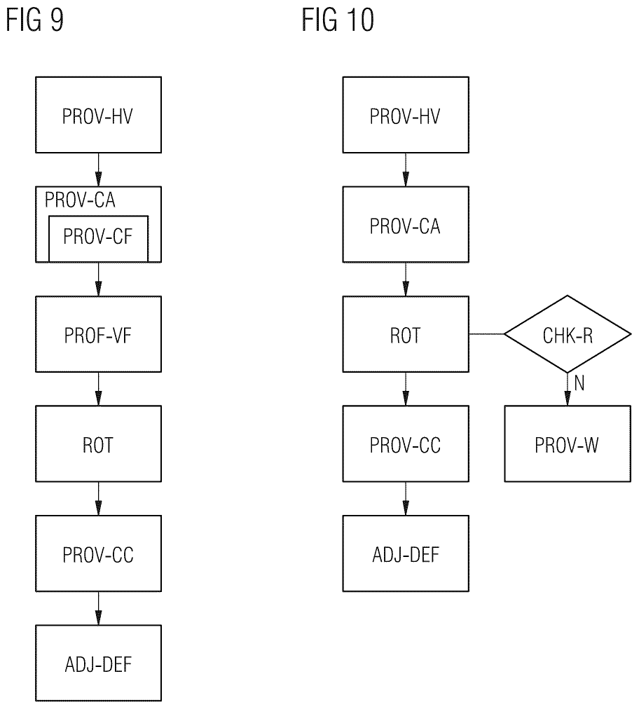

FIG. 9 shows a schematic representation of a further advantageous embodiment of a proposed method for operating a proposed X-ray unit XRU. In this case, the cathode C may have at least one emitter, in particular flat emitter, and a focusing head FH for focusing the electron beam. Energization of the cathode C may involve energizing PROV-CF the at least one emitter. An electrical potential may additionally be provided PROV-VF to the at least one emitter or a further electrical potential to the focusing head FH for focusing the electron beam.

FIG. 10 shows a schematic representation of a further advantageous embodiment of a proposed method for operating a proposed X-ray unit XRU. The specified rotational frequency range may here comprise rotational frequencies from 130 Hz to 160 Hz. It may additionally be monitored CHK-R whether an instantaneous rotational frequency of the anode plate AT is within the specified rotational frequency range. A warning may be provided PROV-W in the negative case N.

The rotational frequency of the anode plate may advantageously be closed-loop controlled within the specified rotational frequency range as a function of an instantaneous temperature of the X-ray tube.

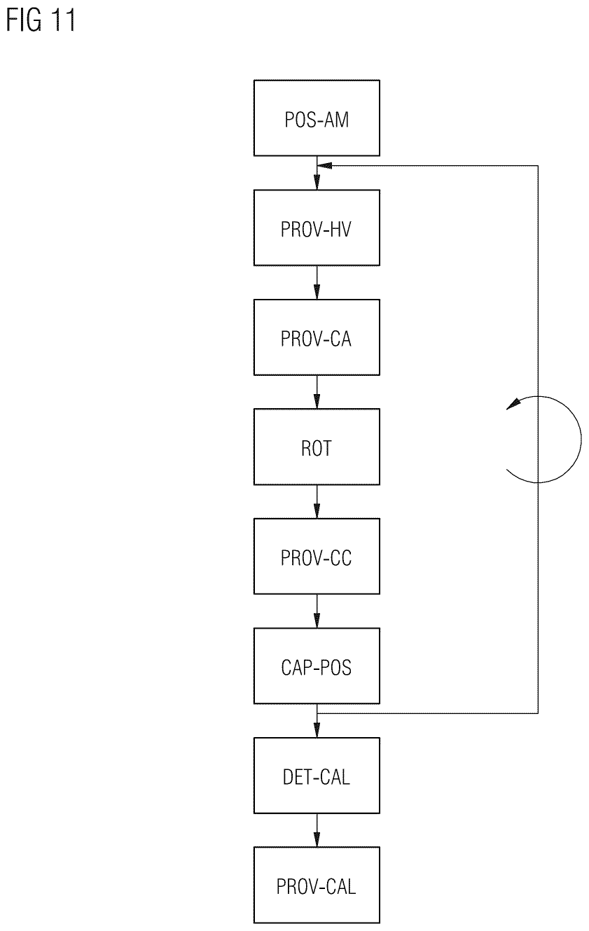

FIG. 11 shows a schematic representation of an advantageous embodiment of a proposed method for calibrating a proposed X-ray unit XRU. In a first step a), an absorption mask may be positioned POS-AM between the beam exit window XRW of the X-ray unit XRU and an X-ray acquisition unit. In a further step b), a high voltage may be provided PROV-HV between the cathode C and the anode plate AT. In a further step c), the cathode C may be energized PROV-CA in order to provide the electron beam. In a further step d), the anode plate AT may be rotated ROT at a rotational frequency within a specified rotational frequency range. In a further step e), the coil CL may be energized PROV-CC with a deflection current in order to provide the magnetic field. In a further step f), a position of a mapping of the absorption mask may be acquired CAP-POS by way of the X-ray acquisition unit. Steps b) to f) may advantageously be carried out repeatedly for a plurality of differing high voltages and/or deflection currents. In this case, a correlation between the deflection of the electron beam and the deflection current and/or the high voltage may be identified DET-CAL on the basis of the plurality of differing high voltages and/or deflection currents and the associated positions of the mapping. In a further step g), an item of calibration information that characterizes the correlation may be provided PROV-CAL.

The X-ray acquisition unit may comprise, for example, an

X-ray camera and/or an X-ray detector. At least four differing high voltages and/or deflection currents may advantageously in each case be specified for repeatedly carrying out of steps b) to f). Identification DET-CAL of the correlation between the deflection of the electron beam and the deflection current and/or high voltage may furthermore involve a respective interpolation.

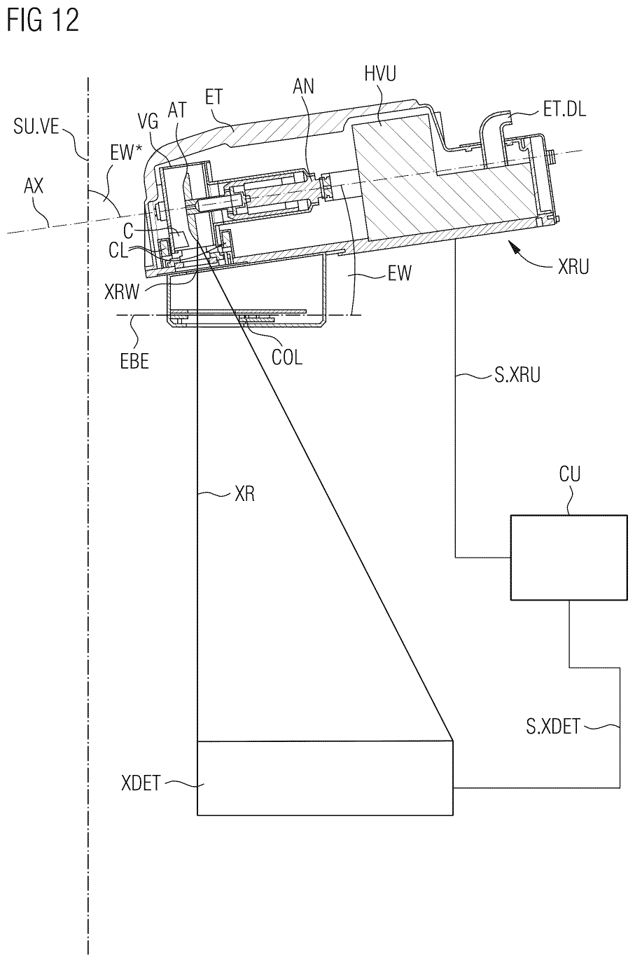

FIG. 12 shows a schematic representation of an advantageous embodiment of a proposed imaging device comprising a proposed X-ray unit XRU, an X-ray detector XDET, and a control unit CU. In this case, the X-ray unit XRU and the X-ray detector XDET may be arranged opposite one another. The X-ray detector XDET may furthermore be configured to acquire X-rays that can be emitted by way of the X-ray unit XRU. The control unit CU may be configured to control the X-ray unit XRU, for example to emit X-rays, by way of a signal S.XRU. The X-ray detector XDET may furthermore be configured to provide a signal S.XDET to the control unit CU as a function of the acquired X-rays. The X-ray unit XRU may advantageously be bearing-mounted so as to be movable relative to the X-ray detector XDET. The control unit CU may furthermore be configured to adapt energization of the coil CL as a function of an instantaneous position of the X-ray unit XRU in such a way that the electron beam that can be emitted by the cathode C is deflected by way of the magnetic field to minimize any movement of the focal spot relative to the X-ray detector XDET.

An operating state of the imaging device is shown schematically in FIG. 12. The X-ray unit XRU may here be operated according to a method for operating an X-ray unit XRU. The X-ray unit XRU may in this case emit X-rays XR to illuminate the X-ray detector XDET.

The imaging device may furthermore comprise a stand unit, and a first and a second holding unit (not shown here). The first holding unit may here be arranged in a rotatably bearing-mounted manner on the stand unit. The X-ray unit XRU may furthermore be arranged on the first holding unit. The second holding unit may additionally be arranged on the stand unit. In this case, the X-ray detector XDET may be arranged on the second holding unit. The first and the second holding units may here hold the X-ray unit and the X-ray detector opposite one another. The X-ray unit XRU may advantageously be movably bearing-mounted parallel to an, in particular vertical, plane SU.VE of the stand unit. In this case, the anode axis AX may be at a predefined angle EW* of less than 90°, in particular 84°, relative to the plane SU.VE of the stand unit SU. The inclination of the anode axis AX relative to the plane SU.VE of the stand unit SU by the specified angle EW* makes it possible to reduce the distance between the focal spot and the thoracic wall of an object under examination. For a specified angle EW* of 84°, the distance between the focal spot and the thoracic wall may be shortened by around 10%.

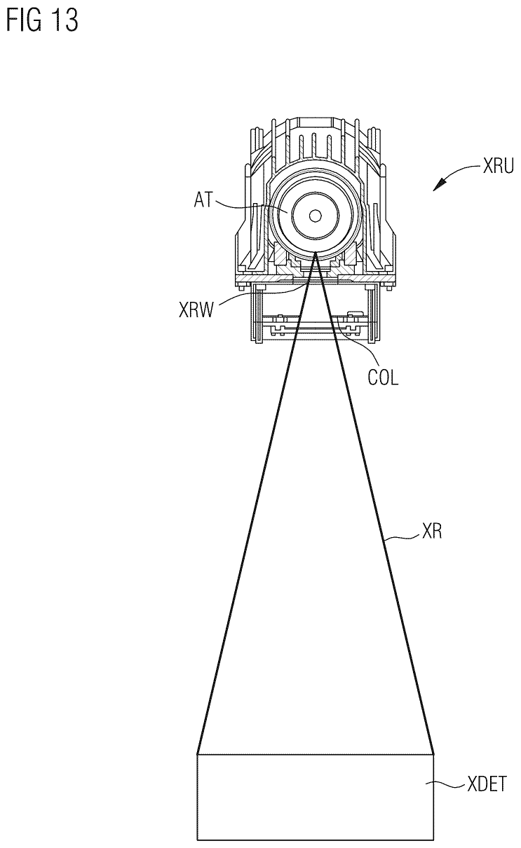

FIG. 13 shows a view of the imaging device shown schematically in FIG. 12 perpendicular to the aperture plane EBE of the aperture unit COL.

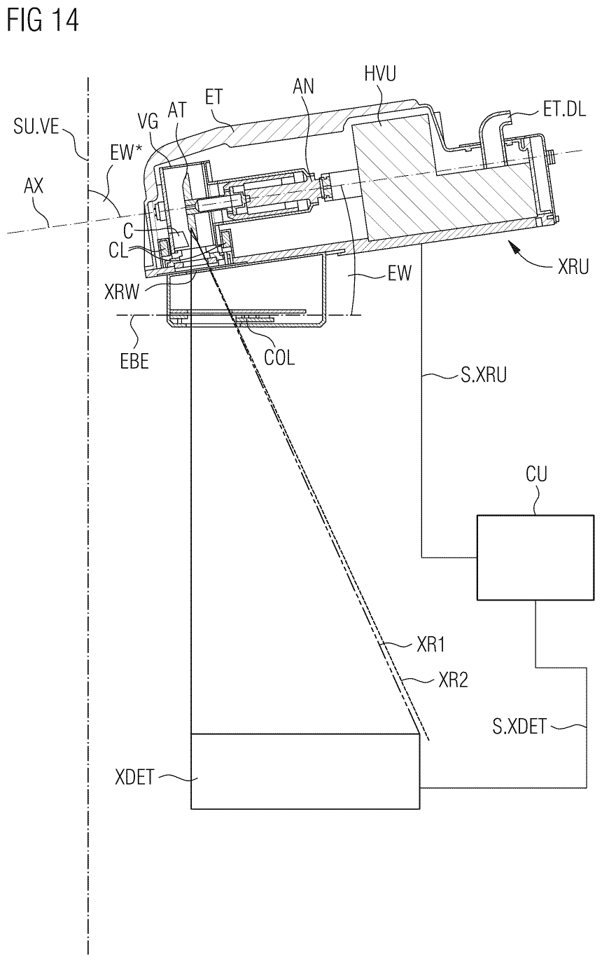

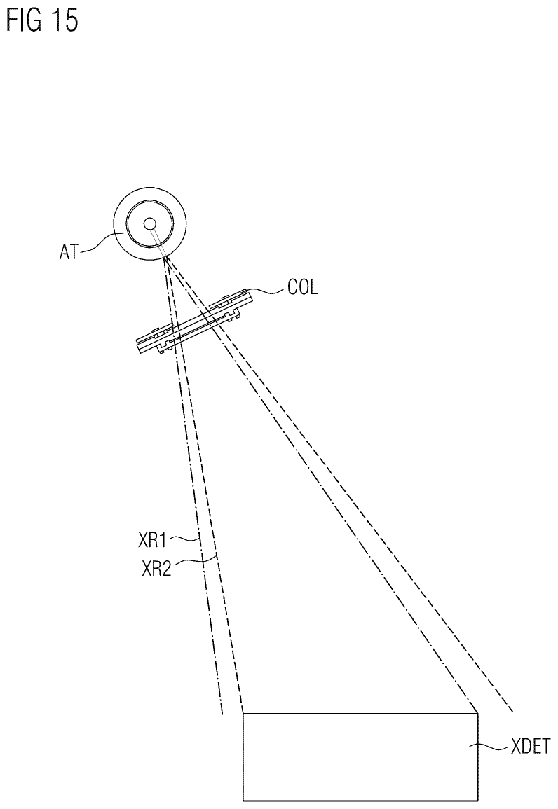

FIGS. 14 and 15 show schematic representations of an advantageous embodiment of the imaging device. In this case, FIGS. 14 and 15 illustrate a profile of the X-rays for two different operating states of the imaging device with respective positioning of the X-ray unit XRU, in particular of the anode plate AT, relative to the X-ray detector. XR1 and XR2 in each case illustrate a spatial profile of the X-rays with the X-ray unit XRU differently positioned relative to the X-ray detector XDET. In both operating states, the control unit CU may here adapt the energization of the coil CL as a function of the respective positioning of the X-ray unit XRU such that the electron beam emitted by the cathode C is deflected by way of the magnetic field to minimize any movement of the focal spot relative to the X-ray detector XDET.

The aperture unit COL may furthermore be adjusted in both operating states such that the respective X-rays XR1 and XR2 partially cross-illuminate the X-ray detector XDET as a function of the deflection of the focal spot.

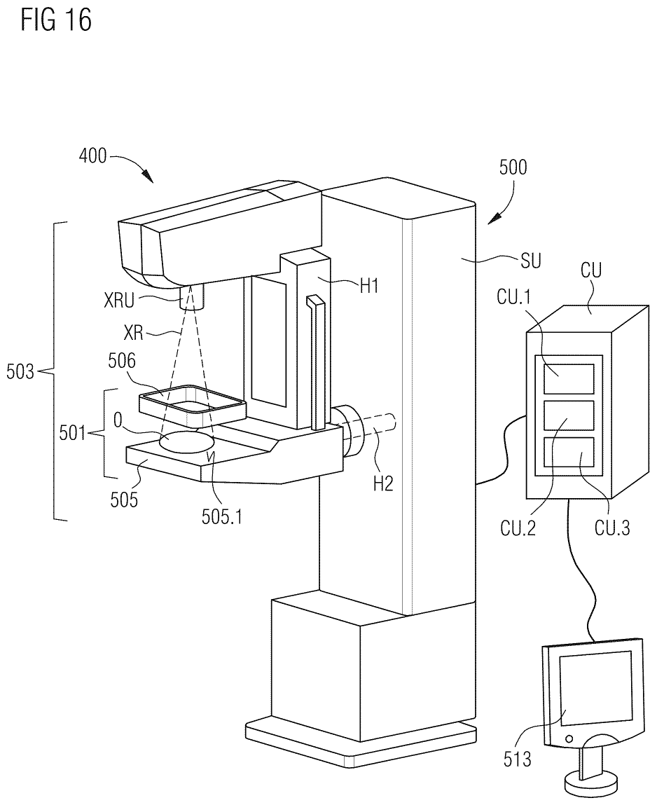

FIG. 16 shows a schematic representation of a further advantageous embodiment of the proposed imaging device. The imaging device may here in particular take the form of a mammography system 500.

FIG. 16 shows the mammography system 500 by way of example and roughly schematically. The mammography system 500 may in particular be configured for tomosynthesis. Relative directions such as “top”, “bottom” etc. relate to the mammography system 500 that has been set up for intended operation. The mammography system 500 may comprise a stand unit SU, a first holding unit H1, and a second holding unit H2. The stand unit SU may comprise, for example, an upright column. The upright column may be floor-mounted. The first holding unit H1 may be arranged in a rotatably bearing-mounted manner on the stand unit SU, in particular the upright column. The X-ray unit XRU may furthermore be arranged on the first holding unit H1. The second holding unit H2 may furthermore be arranged on the stand unit SU, in particular the upright column 507. In this case, the X-ray detector XDET may be arranged on the second holding unit H2. The first and the second holding units may hold the X-ray unit XRU and the X-ray detector XDET opposite one another, in particular in a source-detector arrangement 503. On its surface, in particular on a side facing the X-ray unit XRU, the X-ray detector XDET may comprise a cover XDET.1. The source-detector arrangement 503 may be movably connected to the stand unit SU, such that the height of the cover XDET.1 of the X-ray detector XDET is adjustable to the breast height of an object under examination.

The first holding unit H1 may advantageously be L- or J-shaped and arranged by way of a first leg on the stand unit SU, in particular the column. In this case, the X-ray unit XRU may be arranged on a further leg of the first holding unit H1.

A breast O of the object under examination (shown schematically here) may form an object to be examined and, in embodiments for an examination, be positioned on the cover XDET.1 of the X-ray detector XDET. The cover XDET.1 may form a second compression plate. A compression plate 506 that is movably or displaceably connected to the source-detector arrangement 503 may be arranged over the breast O and the cover XDET.1. For the examination, the breast O may be compressed and simultaneously immobilized by lowering the compression plate 506 onto it, such that pressure is exerted on the breast O between the compression plate 506 and the cover XDET.1. Alternatively, a second compression plate may be used for compression instead of the cover XDET.1 of the X-ray detector XDET such that the breast O is compressed between two compression plates. The two compression plates or the compression plate 506 and the cover XDET.1 of the X-ray detector XDET may be comprised by a compression unit 501. The compression unit 501 may moreover also comprise a displacement unit for displacing the compression plate 506 or compression plates for compression.

The X-ray unit XRU may be arranged relative to the X-ray detector XDET such that the X-ray detector XDET detects X-rays XR emitted by the X-ray unit XRU once at least some of the X-rays XR have passed through the breast O.

In some embodiments, the X-ray source XRU may be movable or rotatable or pivotable relative to the X-ray detector XDET by way of the first holding unit H1, in particular of a rotary arm, for example in a range of ±25° around a base position in which the rotary arm is perpendicular to the cover XDET.1 of the X-ray detector XDET. The X-ray unit XRU may advantageously be movably bearing-mounted parallel to an, in particular vertical, plane of the stand unit, in particular of the column 507. In this case, the anode axis AX of the X-ray unit XRU may be at a predefined angle of less than 90°, in particular 84°, relative to the plane of the stand unit SU.

The mammography system 500 may in particular comprise a control unit CU with an interface CU.1, a computing unit CU.2, and a memory unit CU.3. The control unit CU may in particular be connected to a terminal 513 that for example has a user interface or display unit via which a user may transmit commands to the mammography system 500 or retrieve measurement results, for example the acquired image data, in particular projection captures or X-ray captures. The control unit CU may be located in the same room as the mammography system 500 but may also be located in an adjoining control room or at a still greater spatial distance.

The control unit CU may in particular be a computer, a microcontroller, or an integrated circuit (IC). Alternatively, the control unit CU may be a real or virtual computer network (a technical name for a real computer network is “cluster” and a technical name for a virtual computer network is “cloud”). The control unit CU may be configured as a virtual system that is executed on a computer or a real computer network or a virtual computer network (a technical name is “virtualization”).

The interface CU.1 may be a hardware or software interface (e.g., a PCI bus, CAN bus, USB, or FireWire). The computing unit CU.2 may comprise hardware and/or software components, for example a microprocessor or a field-programmable gate array (FPGA). The memory unit CU.3 may take the form of a volatile working memory (random access memory, RAM) or of a non-volatile mass storage device (hard disk, USB stick, SD card, solid state disk (SSD)).

The interface CU.1 may in particular comprise a plurality of subinterfaces that carry out different method steps of the respective method according to one or more example embodiments of the present invention. In other words, the interface CU.1 may take the form of a plurality of interfaces CU.1. The computing unit CU.2 may in particular comprise a plurality of subcomputing units that carry out different method steps of the respective method according to one or more example embodiments of the present invention. In other words, the computing unit CU.2 may be configured as a plurality of computing units CU.2.

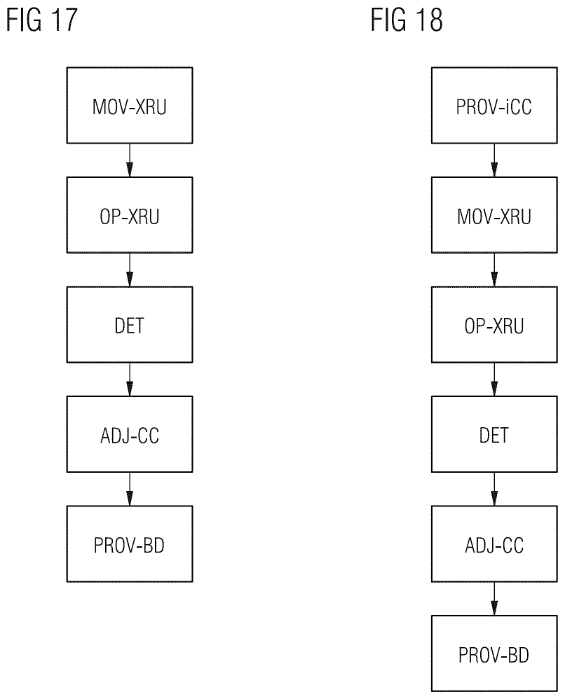

FIG. 17 shows a schematic representation of an advantageous embodiment of a proposed method for acquiring image data by way of a proposed imaging device. The X-ray unit XRU may here be moved MOV-XRU relative to the X-ray detector along a capture path. The capture path may specify one or more capture segments for the arrangement of the X-ray unit XRU. When arranged in the capture segment or in one of the multiple capture segments, the X-ray unit XRU may be operated OP-XRU according to a proposed method for operating an X-ray unit XRU. The X-ray detector XDET may acquire DET the X-rays emitted by way of the X-ray unit XRU. The energization of the coil CL during movement of the X-ray unit XRU along the capture segment or one of the multiple capture segments of the capture path may be adapted ADJ-CC as a function of an instantaneous position of the X-ray unit XRU in such a way that the electron beam emitted by the cathode C is deflected by way of the magnetic field to minimize any movement of the focal spot relative to the X-ray detector. The image data may be provided PROV-BD on the basis of the acquired X-rays.

The coil CL may additionally be energized as a function of a speed of movement of the X-ray unit XRU.

The cathode C may be energized PROV-CA in a pulsed manner to provide the electron beam each time the X-ray unit XRU is arranged in one of the one or more capture segments of the capture path.

In the case of very short pulses, minimization of the movement of the focal spot relative to the X-ray detector may be deactivated. This may be advantageous in specific capture segments of the capture path.

In some cases, in particular in the case of small compression thicknesses of the breast O during a mammography examination, it may possibly not be necessary to minimize the movement of the focal spot relative to the X-ray detector. Minimization may be selectively deactivatable for this purpose.

FIG. 18 shows a schematic representation of a further advantageous embodiment of a proposed method for acquiring image data by way of a proposed imaging device. In this case, the coil may be energized over a predefined period before the high voltage is provided PROV-HV between the cathode C and the anode plate AT and/or the cathode C is energized PROV-CA to provide the electron beam. In particular, an initial deflection current for energizing the coil CL prior to the state of movement MOV-XRU of the X-ray unit XRU may be specified PROV-iCC as a function of the high voltage provided between the cathode C and the anode plate AT and of a planned speed of movement of the X-ray unit XRU.

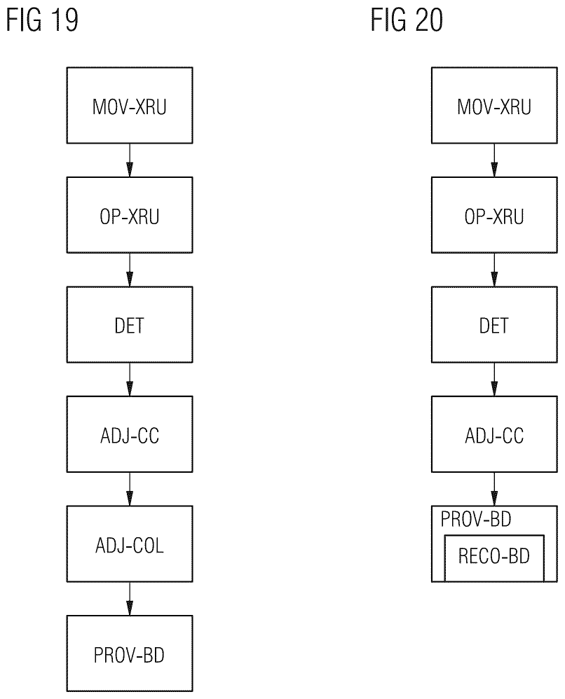

FIG. 19 shows a schematic representation of a further advantageous embodiment of a proposed method for acquiring image data by way of a proposed imaging device. In this case, the X-ray unit XRU may comprise a aperture unit COL that is configured to spatially limit the X-rays exiting from the beam exit window XRW. The aperture unit COL may be adapted ADJ-COL within the one or more capture segments and/or between in each case successive capture segments of the capture path of the X-ray unit XRU. The aperture unit COL may be adapted ADJ-COL within the one or more capture segments of the capture path contrary to a direction of movement of the X-ray unit XRU. The aperture unit COL may furthermore be adapted ADJ-COL between in each case successive capture segments of the capture path along a direction of movement of the X-ray unit XRU.

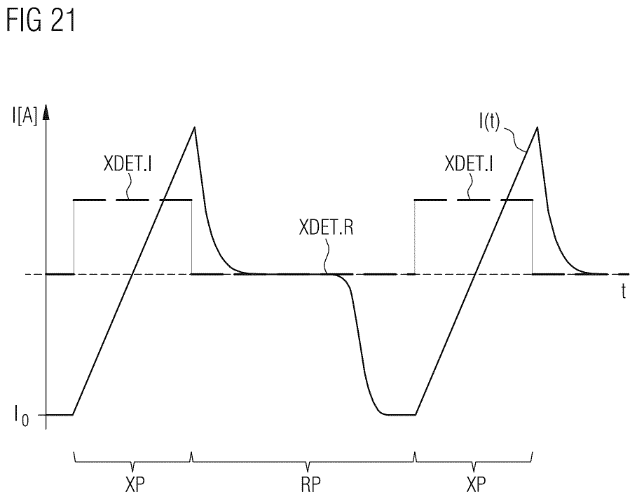

FIG. 20 shows a schematic representation of a further advantageous embodiment of a proposed method for acquiring image data by way of a proposed imaging device. In this case, provision of the image data PROV-BD may involve reconstructing RECO-BD the image data on the basis of the acquired X-rays and of information relating to the capture segment or the respective capture segments of the multiple capture segments of the X-ray unit XRU relative to the X-ray detector XDET.

FIG. 21 shows a schematic representation of an exemplary deflection current-time curve I(t). The graph shown in FIG. 21 illustrates two main cycles of the operation of the X-ray unit XRU, an X-ray radiation phase XP and a readout phase RP.

In the X-ray radiation phase XP, the X-ray unit XRU may be moved along a capture segment of the capture path. In this case, the X-ray unit XRU may be operated according to a proposed method for operating an X-ray unit XRU and emit X-rays. During the X-ray radiation phase, the X-ray detector XDET may acquire the X-rays emitted by the X-ray unit XRU. The X-ray detector XDET may be operated for this purpose for example in an integrating mode XDET.I and integrate the incident X-rays. During the X-ray radiation phase RP, the deflection current I may rise in the coil starting from an initial deflection current I0. The initial deflection current I0 may be dependent on the high voltage and an, in particular planned, speed of movement of the X-ray unit XRU. The rise in deflection current may here be dependent on a speed of movement of the X-ray unit XRU. In this way, the deflection of the electron beam may advantageously compensate the movement of the X-ray unit XRU relative to the X-ray detector XDET.

Advantageously, no deflection current flows through the coil in the readout phase RP. The X-ray detector XDET may be read out during the readout phase RP. The X-ray detector XDET may be operated for this purpose in a readout mode XDET.R.

Shortly prior to the start of the next X-ray radiation phase XP, the coil may be energized with the initial deflection current I0.

In one embodiment, the electron beam may be deflected asymmetrically per capture. This may result in the focal spot or its mapping on the beam exit window XRW not remaining static.

The deflection of the electron beam may be dependent on the high voltage and the energization of the cathode. In the case of different capture durations, deflection may be terminated early such that the entire current range of the deflection, in particular of the deflection current, is not passed through.

Closed-loop control of the deflection may advantageously not be directed toward a static position of the focal spot on the beam exit window XRW. Instead, intentional blurring of the focal spot may be produced on the X-ray detector XDET during the X-ray radiation phase XP, wherein the focal spot geometry may change from “small” to “normal”. This may result in a dynamic gain in the modulation transfer function (MTF) and resolution in the individual captures.

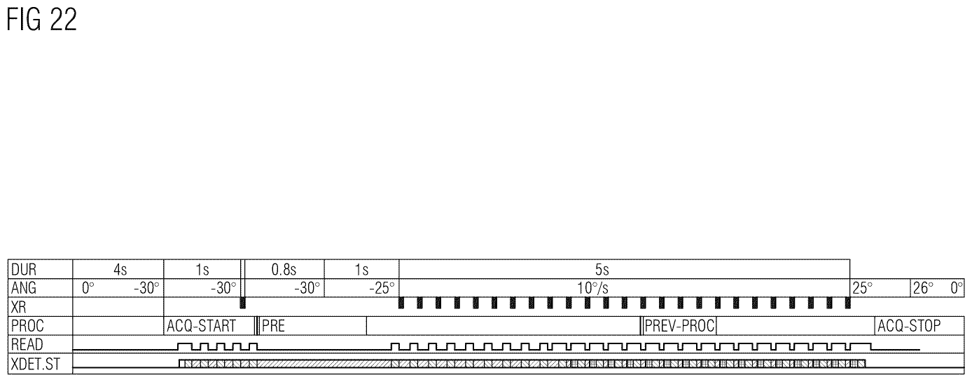

FIG. 22 shows a schematic representation of an exemplary sequence diagram for use in a proposed method for acquiring image data by way of a proposed imaging device, in particular a tomosynthesis scan with automatic exposure control at an image capture rate of five images per second.

The first line DUR of the sequence diagram represents the respective exemplary durations for each phase shown. A further line ANG of the sequence diagram states an angle between the X-ray unit XRU and the X-ray detector XDET. The exemplary sequence begins with a four-second movement of the X-ray unit XRU to a position to a normal of an, in particular X-ray-sensitive, surface of the X-ray detector XDET facing the X-ray unit. An angle between the X-ray unit XRU and the X-ray detector XDET may here be modified from 0° to −30°.

A further line XR of the sequence diagram schematically shows the respective emission of X-rays by the X-ray unit XRU.

A short waiting time for automatic exposure control may follow after a one-second preparatory phase for the X-ray detector XDET and the high-voltage unit HVU. A tomosynthesis speed may then be selected and movement of the X-ray unit XRU relative to the X-ray detector XDET started. The actual tomosynthesis scan may last, for example, five seconds, during which the angle between the X-ray unit XRU and the X-ray detector XDET is modified from −25° to +25°. The tomosynthesis scan may advantageously start when the X-ray unit XRU is at an angle of −25° relative to the X-ray detector XDET.

During the tomosynthesis scan, multiple X-ray pulses, in particular pulses of X-ray radiation, may be triggered, in particular by way of pulsed energization of the cathode C.

A further line PROC of the sequence diagram illustrates various image processing steps. Capture may be started in a first step ACQ-START. Preliminary captures may initially be acquired here. The preliminary captures may be analyzed, for example for automatic exposure control (AEC), in a further step PRE. During capture of the image data, the image data so far captured may be processed for a preview in a further step PREV-PROC. The acquired image data may be provided for image processing and capture stopped in a further step ACQ-STOP.

A further READ line of the sequence diagram illustrates a

READ signal on the X-ray detector XDET, which clarifies continuous data acquisition during the tomosynthesis scan. A further line XDET.ST of the sequence diagram shows a status, in particular operating mode, of the X-ray detector XDET. The status of the X-ray detector XDET may change between idle, integration and readout, which shows the various operating states of the X-ray detector XDET during the scan process.

The schematic representations contained in the described figures do not depict any scale or size ratios.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

It should finally once again be noted that the methods described above in detail and the depicted apparatuses are merely exemplary embodiments which may be modified in the most varied manner by a person skilled in the art without departing from the scope of the present invention. Furthermore, use of the indefinite article “a” does not rule out the possibility of a plurality of the features in question also being present. Likewise, the terms “unit” and “element” do not rule out the possibility of the components in question consisting of multiple interacting sub-components which may optionally also be spatially distributed.

In the context of the present application, the expression “on the basis of” may in particular be taken to mean “using”. In particular, wording according to which a first feature is generated (or: ascertained, determined etc.) on the basis of a second feature does not rule out the possibility of the first feature being generated (or: ascertained, determined etc.) on the basis of a third feature.

What is claimed is:

1. A method for acquiring image data via an imaging device, the method comprising:
moving an X-ray unit along a capture path relative to an X-ray detector, wherein the capture path specifies one or more capture segments for arranging the X-ray unit; and
operating the X-ray unit when arranged in a capture segment among the one or more capture segments, wherein operating of the X-ray unit includes
providing a high voltage between a cathode and an anode plate,
energizing the cathode to provide an electron beam,
rotating the anode plate at a rotational frequency within a specified rotational frequency range,
energizing a coil with a deflection current to provide a magnetic field, and
adapting deflection of the electron beam by adapting energizing of the coil;
acquiring, via the X-ray detector, X-rays emitted by the X-ray unit, wherein
energizing of the coil during movement of the X-ray unit along the capture segment is adapted as a function of an instantaneous position of the X-ray unit such that the electron beam emitted by the cathode is deflected by the magnetic field to minimize movement of a focal spot relative to the X-ray detector,
at least one of (i) the coil is energized over a first period before the high voltage is provided between the cathode and the anode plate, or (ii) the cathode is energized to provide the electron beam; and
providing the image data based on acquired X-rays, wherein
the X-ray unit has an X-ray tube including
the cathode,
a rotating anode including the anode plate rotatably bearing-mounted about an anode axis, and
a vacuum housing, wherein
the cathode and the rotating anode are arranged within the vacuum housing, and
the coil arranged outside the vacuum housing, wherein
the coil and the X-ray tube are electrically isolated from one another,
the coil is configured to generate the magnetic field to permeate an interspace between the cathode and the anode plate,
the magnetic field is configured to deflect the electron beam originating from the cathode and impinging on the anode plate in the focal spot of the X-ray tube,
the vacuum housing has a beam exit window on a side arranged radially relative to the anode axis, and
the beam exit window is configured to allow passage of X-rays originating from the focal spot on the anode plate.

2. The method as claimed in claim 1, wherein
the X-ray unit includes an aperture unit configured to spatially limit the X-rays exiting from the beam exit window, and
the aperture unit is adapted at least one of (i) within the one or more capture segments, or (ii) between successive capture segments of the capture path of the X-ray unit.

3. The method as claimed in claim 2, wherein, within the one or more capture segments of the capture path, the aperture unit is adapted contrary to a direction of movement of the X-ray unit.

4. The method as claimed in claim 2, wherein, between successive capture segments of the capture path, the aperture unit is adapted along a direction of movement of the X-ray unit.

5. The method as claimed in claim 1, wherein the coil is additionally energized as a function of a speed of movement of the X-ray unit.

6. The method as claimed in claim 1, wherein an initial deflection current for energizing the coil prior to a start of movement of the X-ray unit is specified as a function of
the high voltage provided between the cathode and the anode plate, and
a planned speed of movement of the X-ray unit.

7. The method as claimed in claim 1, wherein the cathode is energized in a pulsed manner to provide the electron beam each time the X-ray unit is arranged in one of the one or more capture segments of the capture path.

8. The method as claimed in claim 1, wherein providing of the image data includes
reconstructing the image data based on the acquired X-rays and information relating to the capture segment or respective capture segments of multiple capture segments of the X-ray unit relative to the X-ray detector.

9. The method as claimed in claim 1, wherein the X-ray unit further comprises:
a leak-tight single-tank housing, wherein
the X-ray tube and the coil are arranged within the leak-tight single-tank housing, and
the leak-tight single-tank housing is filled with at least one of a cooling medium or an insulating medium.

10. The method as claimed in claim 1, wherein the X-ray unit comprises:
an aperture unit configured to spatially limit the X-rays exiting through the beam exit window.

11. The method as claimed in claim 10, wherein the anode axis has a specified angle relative to an aperture plane of the aperture unit.

12. The method as claimed in claim 1, wherein the coil has a coil geometry that is adapted to a geometry of the X-ray tube.

13. The method as claimed in claim 12, wherein
the coil geometry includes a cross-sectional geometry of the coil, and
the cross-sectional geometry of the coil is adapted, at least on an inner side of the coil, to a geometry of the vacuum housing within an opening region of the coil.

14. The method as claimed in claim 1, wherein the electron beam is at least partly within an opening region of the coil when the X-ray unit is in an operating state.

15. The method as claimed in claim 14, wherein the focal spot of the X-ray tube is within the opening region of the coil when the X-ray unit is in the operating state.

16. The method as claimed in claim 1, wherein the coil is an air-core coil.

17. The method as claimed in claim 1, wherein the coil and the electron beam are isolated from one another by a vacuum housing wall portion formed from a non-magnetic metallic material of low electrical conductivity.

18. The method as claimed in claim 1, wherein the cathode has a focusing head configured to focus the electron beam.

19. The method as claimed in claim 18, wherein the focusing head is configured, at least by shape, to electrostatically focus the electron beam.

20. The method as claimed in claim 1, wherein
the cathode has at least one emitter and a focusing head to focus the electron beam,
energizing of the cathode includes energizing the at least one emitter, and
the method further includes providing an electrical potential of the at least one emitter, or a further electrical potential, to the focusing head to focus the electron beam.

21. A non-transitory computer-readable medium storing a computer program that, when executed by a control unit at an X-ray unit, causes the X-ray unit to perform the method of claim 13.
